(19) United States
(12) Patent Application Publication
(10) Pub. No.: US 2026/0258046 A1
(43) Pub. Date: Sep. 3, 2026

(54) METHODS OF PREPARING SUBSTITUTED PYRAZOLOPYRIMIDINES

(71) Applicant: KSQ Therapeutics, Inc., Lexington, MA (US)

(72) Inventor: Jeremy Clinton WILT, Needham, MA (US)

(21) Appl. No.: 18/717,852

(22) PCT Filed: Dec. 8, 2022
(86) PCT No.: PCT/US2022/081150

§ 371 Date: Jun. 7, 2024

Related U.S. Application Data
(60) Provisional application No. 63/287,623, filed on Dec. 9, 2021.

(51) Int. Cl. C07D 487/04; C07D 233/90; C07D 403/12; C07D 403/14

(52) U.S. Cl. CPC C07D 487/04; C07D 233/90; C07D 403/12; C07D 403/14

(57) ABSTRACT

The present disclosure provides compounds having Formula I: and the pharmaceutically acceptable salts and solvates thereof, wherein R1 is defined as set forth in the specification. The present disclosure also provides methods of preparing a compound of Formula (VI):

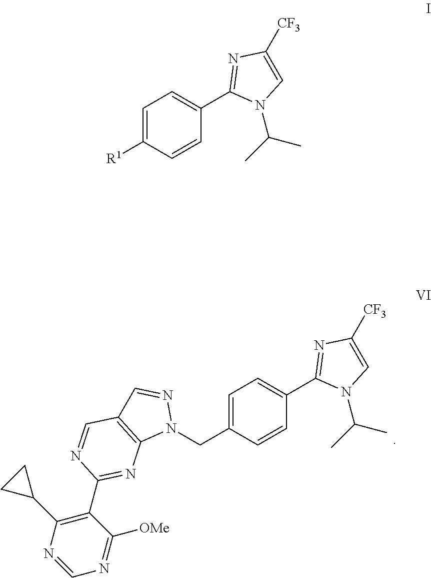

### CROSS REFERENCE TO RELATED APPLICATIONS

This application claims the priority benefit of U.S. Provisional Application No. 63/287,623, filed Dec. 9, 2021, which is hereby incorporated by reference herein in its entirety.

### FIELD OF THE INVENTION

The present disclosure relates to methods for preparing substituted pyrazolopyrimidines and substituted purines, which can be used as ubiquitin-specific-processing protease 1 (USP1) inhibitors.

### BACKGROUND

Substituted pyrazolopyrimidines, such as 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine are inhibitors of USP1.

In addition, the isolation and commercial-scale preparation of substituted pyrazolopyrimidines, such as 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine present a number of challenges. For example, methods requiring costly purification methods, such as chromatography, produce significant amounts of impurities, or utilize large amounts of costly and undesirable reagents, are often unsuitable for commercial scale production.

The compound 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine is disclosed in International Publication No. WO 2020/132269. Solid state forms of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine and its pharmaceutically acceptable salts and co-crystals are disclosed in International Application No. PCT/US2021/057072. Methods to treat cancer with 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine are disclosed in International Publication No. WO2021/163530. A method for the synthesis of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine is disclosed in WO 2020/132269 and is illustrated below.

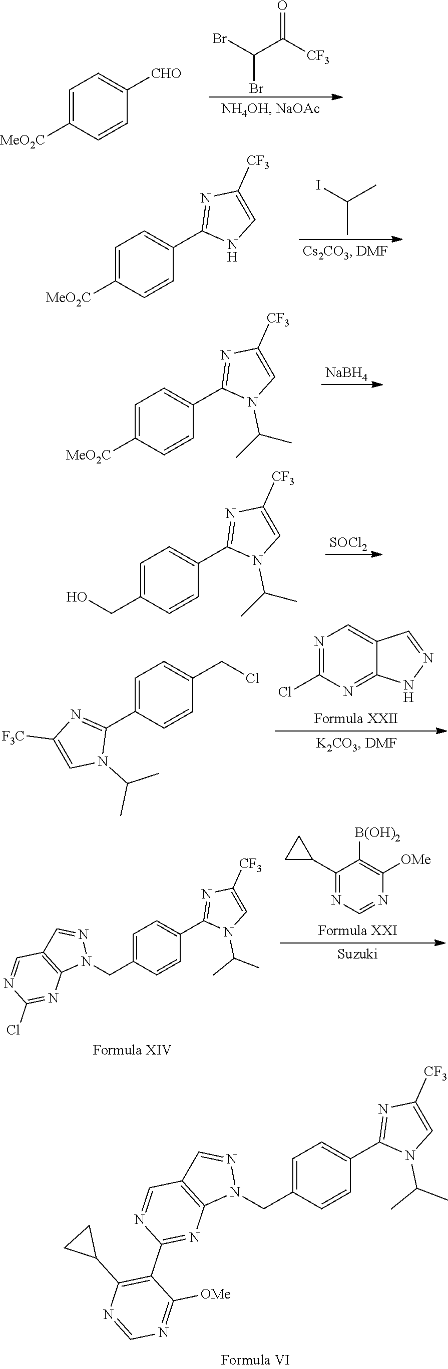

While this method allows for small scale preparation of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine, it suffers from poor selectivity in the alkylation reaction to form the compound of Formula XIV, resulting in a significant yield loss at a late stage of the synthesis. Separation of the resulting N1 and N2 regioisomers requires chromatography, which is time consuming and inefficient on large, kilogram scale. Additionally, intermediate XXII is not readily commercially available to support large, kilogram scale preparation.

Accordingly, there is a current need for one or more methods for preparing substituted pyrazolopyrimidines, such as 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine that avoid costly and undesirable challenges.

### BRIEF SUMMARY

Disclosed herein are improved processes for preparing 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine, as well as novel intermediates useful for its synthesis. These methods and novel intermediates enable efficient manufacturing of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine on large, kilogram scale.

Applicant has discovered reaction pathways for the synthesis of substituted pyrazolopyrimidines, such as 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine, using intermediates and processes that significantly reduce the number of steps necessary to synthesize said substituted pyrazolopyrimidines. In addition, Applicant has discovered several intermediate compounds (also referred to herein as Compounds of the Disclosure) that allow for a more efficient process for prepaing these substituted pyrazolopyrimidines, such as 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine.

Furthermore, Applicant has discovered a reaction pathway to prepare a compound of Formula XIV:

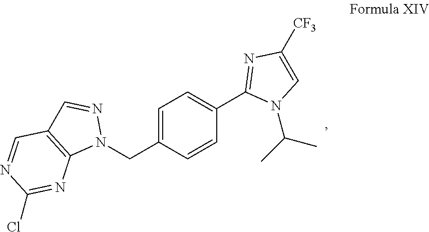 [SMILES: CC(C)n1cc(C(F)(F)F)nc1-c1ccc(Cn2ncc3cnc(Cl)nc32)cc1]

that results in minimal amounts (e.g., less than about 5% AUC as compared to the area of the compound of Formula XIV) of an impurity compound of Formula XVII:

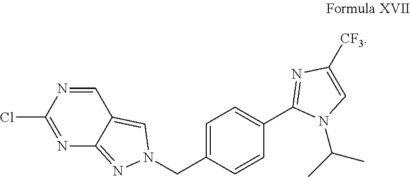 [SMILES: CC(C)n1cc(C(F)(F)F)nc1-c1ccc(Cn2cc3cnc(Cl)nc3n2)cc1]

In one aspect, the present disclosure relates to a Compound of the Disclosure that is a compound, or a pharmaceutically acceptable salt or solvate thereof, having Formula I:

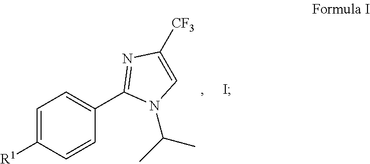

wherein:
R¹ is selected from cyano, —CHO, —CH₂NR²R³, and —CH₂R⁴;
R² is selected from hydrogen, optionally substituted aryl, and optionally substituted heteroaryl;
R³ is selected from hydrogen, amino, and —NR⁵R⁶;
R⁴ is selected from Formula II and Formula III:

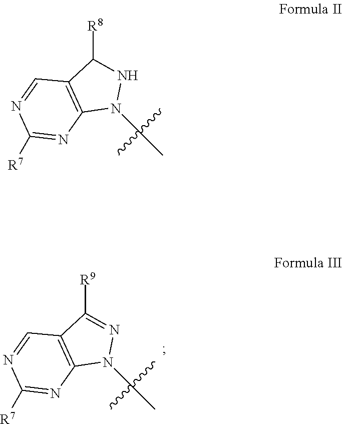

R⁵ and R⁶ are individually selected from hydrogen and an amine protecting group;
R⁷ is selected from optionally substituted aryl and optionally substituted heteroaryl;
R⁸ is selected from carbonyl, alkoxy, and sulfonate; and
R⁹ is selected from hydroxy, alkoxy, sulfonate, and a leaving group.

In some embodiments, a Compound of the Disclosure is a compound having Formula I, wherein one of R⁵ and R⁶ is hydrogen and the other of R⁵ and R⁶ is an amine protecting group selected from t-butyloxycarbonyl (Boc), fluorenylmethoxycarbonyl (Fmoc), benzyloxycarbonyl (Cbz), acetyl, trifluoroacetamide, phthalimide, benzyl, trityl, benzylideneamine, and toluenesulfonate. In some embodiments, the amine protecting group is t-butyloxycarbonyl (Boc).

In some embodiments, a Compound of the Disclosure is a compound having Formula I, wherein R¹ is —CH₂NR²R³, R² is hydrogen, and R³ is amino.

In some embodiments, a Compound of the Disclosure is a compound having Formula I, wherein R¹ is —CH₂NR²R³ and R² is an optionally substituted pyrimidinyl.

In some embodiments, a Compound of the Disclosure is a compound having Formula I, wherein R⁷ has a Formula XIX:

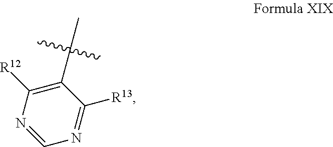

wherein R¹² and R¹³ are individually selected from hydrogen, hydroxy, alkyl, alkoxy, and cycloalkyl. In some embodiments, R¹² is cyclopropyl and R¹³ is methoxy.

In some embodiments, a Compound of the Disclosure is a compound having Formula I, wherein the leaving group is selected from trifluoromethanesulfonate, toluenesulfonate, methanesulfonate, and halogen. In some embodiments, the leaving group is trifluoromethanesulfonate.

In some embodiments, a Compound of the Disclosure is a compound having Formula I, selected from the group consisting of:

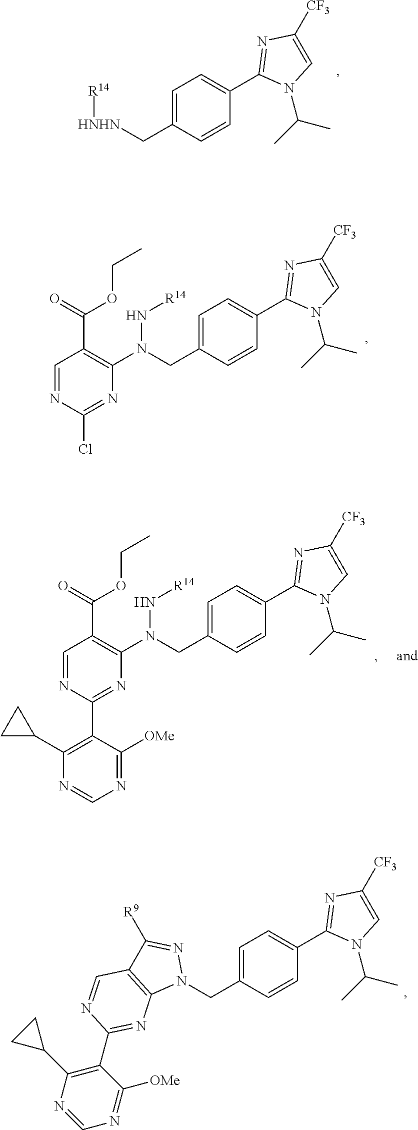

wherein R⁹ is a leaving group, and wherein R¹⁴ is an amine protecting group.

In some embodiments, a Compound of the Disclosure is a compound having Formula I, selected from the group consisting of:

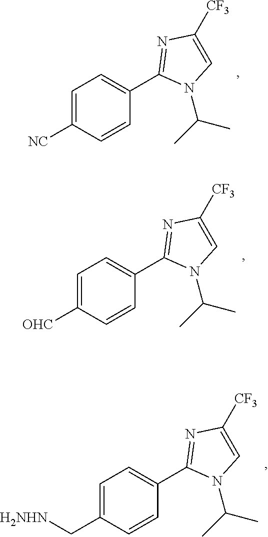

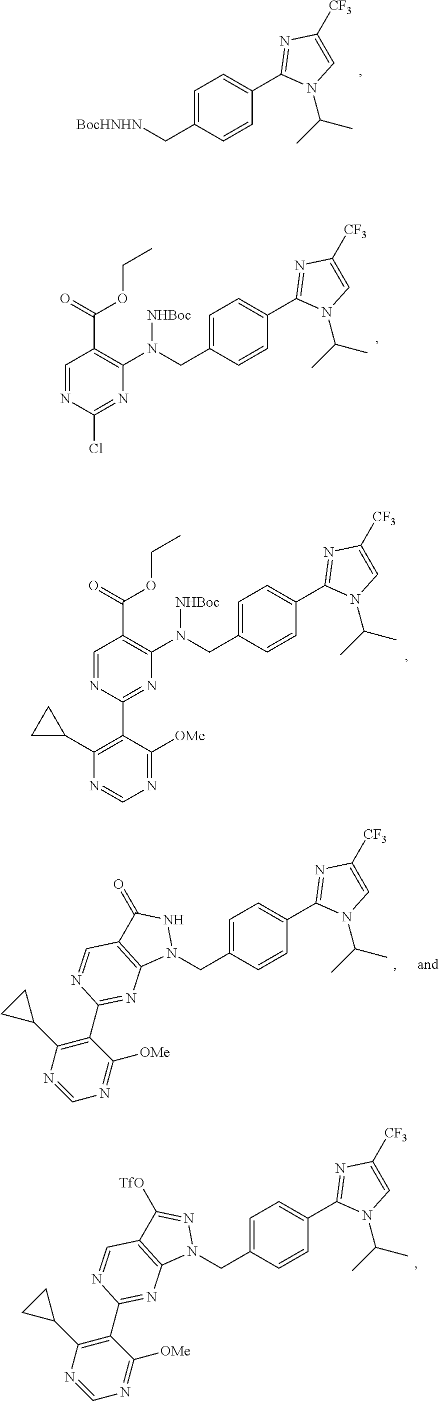

or a pharmaceutically acceptable salt thereof.

In another aspect, the present disclosure relates to a Compound of the Disclosure that is a compound, or a pharmaceutically acceptable salt or solvate thereof, having Formula IV:

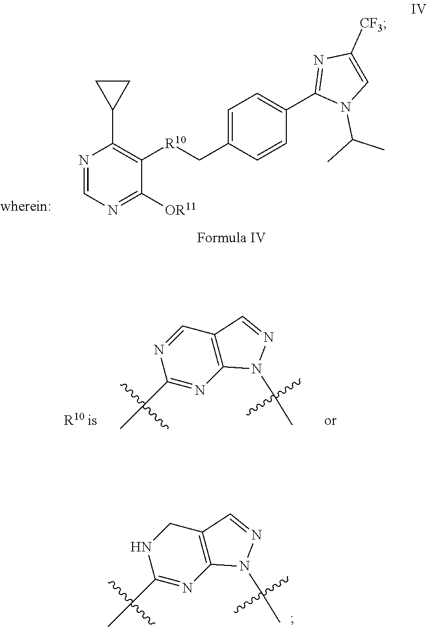

and
R¹¹ is hydrogen or alkyl.

In some embodiments, a Compound of the Disclosure is a compound having Formula IV, selected from the group consisting of:

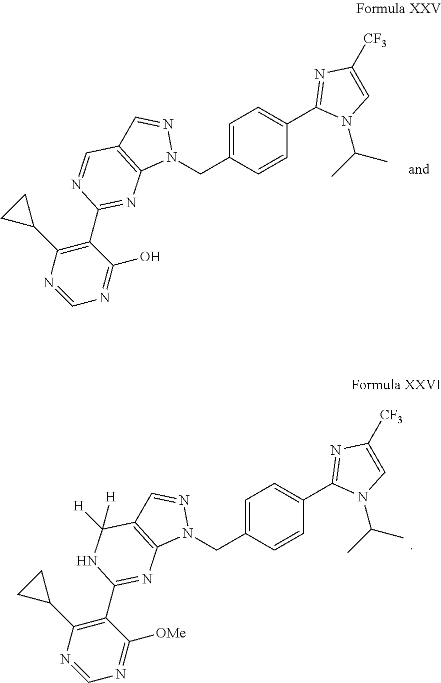 [SMILES: CC(C)n1cc(C(F)(F)F)nc1-c1ccc(Cn2ncc3cnc(-c4c(O)ncnc4C4CC4)nc32)cc1 ; COc1ncnc(C2CC2)c1C1=Nc2c(cnn2Cc2ccc(-c3nc(C(F)(F)F)cn3C(C)C)cc2)CN1]

In another aspect, the present disclosure relates to a Compound of the Disclosure that is a compound, or a salt or solvate thereof, having Formula XIV:

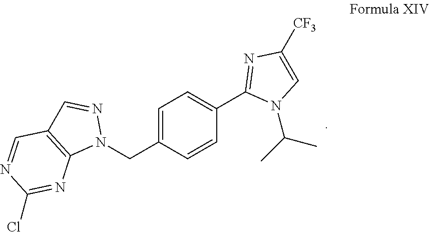 [SMILES: CC(C)n1cc(C(F)(F)F)nc1-c1ccc(Cn2ncc3cnc(Cl)nc32)cc1]

In another aspect, the present disclosure relates to a Compound of the Disclosure that is a compound, or a salt or solvate thereof, having Formula XVII:

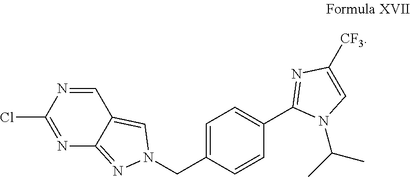 [SMILES: CC(C)n1cc(C(F)(F)F)nc1-c1ccc(Cn2cc3cnc(Cl)nc3n2)cc1]

In another aspect, the present disclosure relates to a Compound of the Disclosure that is a compound, or a salt or solvate thereof, having Formula XX:

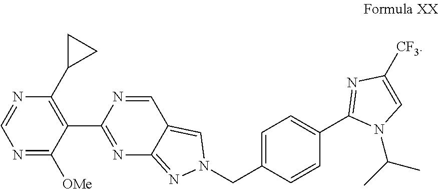 [SMILES: COc1ncnc(C2CC2)c1-c1ncc2cn(Cc3ccc(-c4nc(C(F)(F)F)cn4C(C)C)cc3)nc2n1]

In another aspect, the present disclosure relates to a Compound of the Disclosure that is a compound, or a salt or solvate thereof, having Formula XXVIII:

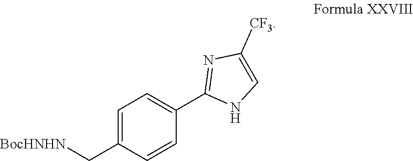

In another aspect, the present disclosure relates to a Compound of the Disclosure that is a compound, or a salt or solvate thereof, having Formula XXVIV:

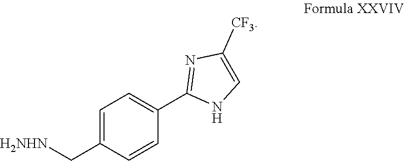

In another aspect, the present disclosure relates to a Compound of the Disclosure that is a compound, or a salt or solvate thereof, having Formula XXX:

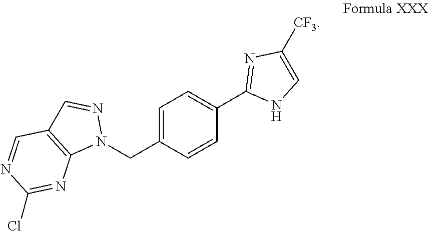 [SMILES: FC(F)(F)c1c[nH]c(-c2ccc(Cn3ncc4cnc(Cl)nc43)cc2)n1]

In another aspect, the present disclosure relates to a process for preparing a compound of Formula VIb:

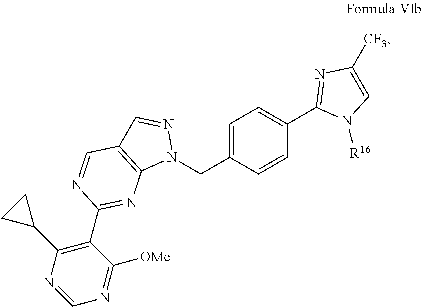

comprising reducing a compound of Formula Vb:

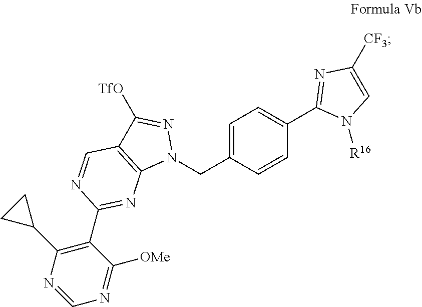

wherein R¹⁶ is selected from hydrogen, alkyl, and an amine protecting group.

In some embodiments of the process, R¹⁶ is hydrogen. In some embodiments, R¹⁶ is an amine protecting group. In some embodiments, R¹⁶ is alkyl. In some embodiments, R¹⁶ is (C₁-C₅) alkyl. In some embodiments, R¹⁶ is methyl. In some embodiments, R¹⁶ is ethyl. In some embodiments, R¹⁶ is n-propyl. In some embodiments, R¹⁶ is isopropyl.

In some embodiments of the process, Formula VIb is Formula VI: Formula VI.

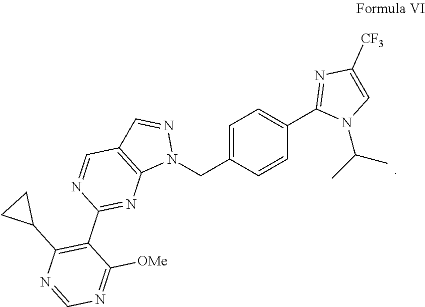 [SMILES: COc1ncnc(C2CC2)c1-c1ncc2cnn(Cc3ccc(-c4nc(C(F)(F)F)cn4C(C)C)cc3)c2n1]

In some embodiments of the process, Formula Vb is Formula V:

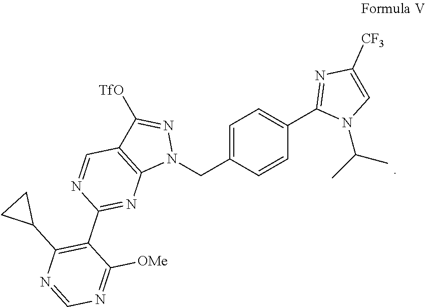 [SMILES: COc1ncnc(C2CC2)c1-c1ncc2c(OS(=O)(=O)C(F)(F)F)nn(Cc3ccc(-c4nc(C(F)(F)F)cn4C(C)C)cc3)c2n1]

In some embodiments, said reducing occurs in the presence of a palladium catalyst and a trialkylsilane.

In some embodiments, said reducing occurs in the presence of a palladium catalyst and triethylsilane.

In some embodiments, said reducing occurs in the presence of a palladium catalyst, triethylsilane, and a base. In some embodiments, the base is triethylamine.

In another aspect, the present disclosure relates to a process comprising mixing the compound of Formula VI with gentisic acid in a solvent to form a compound of Formula XVIII.

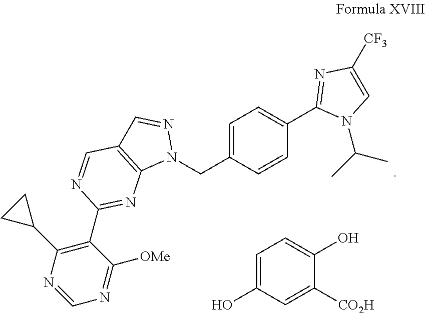 [SMILES: COc1ncnc(C2CC2)c1-c1ncc2cnn(Cc3ccc(-c4nc(C(F)(F)F)cn4C(C)C)cc3)c2n1 ; O=C(O)c1cc(O)ccc1O]

In some embodiments, the compound of Formula XVIII is a co-crystal.

In some embodiments, the compound of Formula VI is mixed with gentisic acid in the presence of a solvent. In some embodiments, the solvent is a mixture of isopropanol and heptane.

In some embodiments, the compound of Formula XVIII is isolated from the solvent. In some embodiments, the compound of Formula XVIII is isolated from the solvent by precipitation. In some embodiments, the compound of Formula XVIII is isolated from the solvent by evaporation. In some embodiments, the compound of Formula XVIII is isolated from the solvent by crystalization. In some embodiments, the compound of Formula XVIII is re-crystallized using one or more solvents.

In another aspect, the present disclosure relates to a process for preparing a compound of Formula Vab:

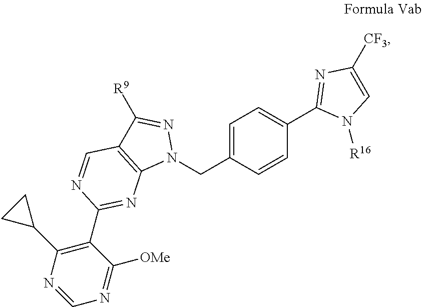

wherein R⁹ is a leaving group selected from trifluoromethanesulfonate, toluenesulfonate, methanesulfonate;
comprising reacting a compound of Formula VIIb:

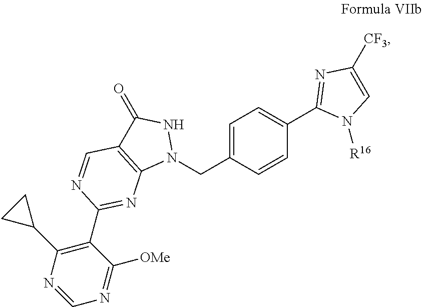

with toluenesulfonic anhydride, toluenesulfonyl chloride, methanesulfonic anhydride, methanesulfonyl chloride, trifluoromethanesulfonic anhydride, or trifluoromethanesulfonyl chloride to form a compound of Formula Vab.

In some embodiments, Formula Vab is Formula Va:

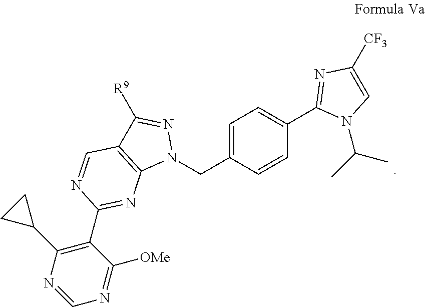

In some embodiments, Formula VIIb is Formula VII:

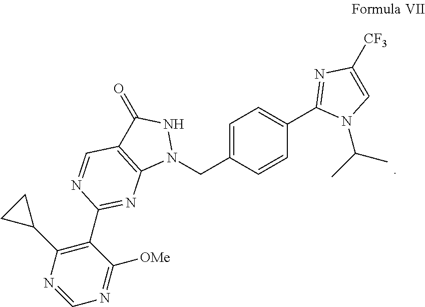 [SMILES: COc1ncnc(C2CC2)c1-c1ncc2c(=O)[nH]n(Cc3ccc(-c4nc(C(F)(F)F)cn4C(C)C)cc3)c2n1]

In some embodiments, the compound of Formula VII is reacted with trifluoromethanesulfonic anhydride or trifluoromethanesulfonyl chloride.

In another aspect, the present disclosure relates to a process for preparing a compound of Formula VIIb:

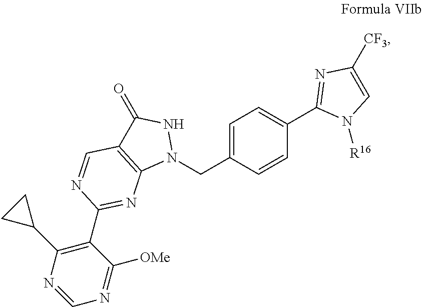

comprising reacting a compound of Formula VIIIb:

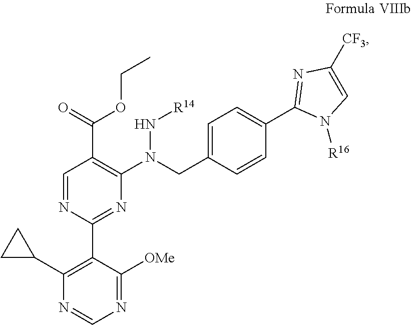

or a salt thereof, with an acid to form a compound of Formula VIIb, wherein R¹⁴ is an amine protecting group. In some embodiments, the acid is trifluoroacetic acid.

In some embodiments, Formula VIIIb is Formula VIII:

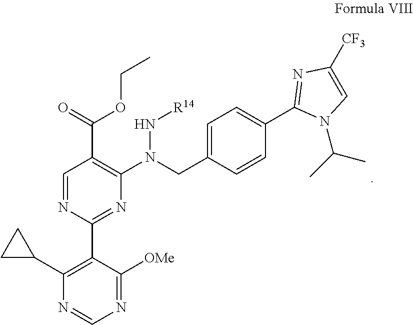

In some embodiments, said reacting occurs in the presence of toluene as a solvent. In some embodiments, the compound of Formula VII or VIIb is isolated from the solvent. In some embodiments, the compound of Formula VII or VIIb is isolated from the solvent by precipitation. In some embodiments, the compound of Formula VII or VIIb is isolated from the solvent by evaporation. In some embodiments, the compound of Formula VII or VIIb is isolated from the solvent by crystalization.

In some embodiments, said reacting occurs at a temperature of from about 20° C. to about 55° C.

In another aspect, the present disclosure relates to a process for preparing a compound of Formula VIIIb:

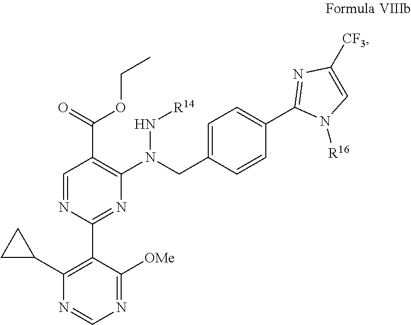

or a salt thereof, comprising coupling a compound of Formula IXb:

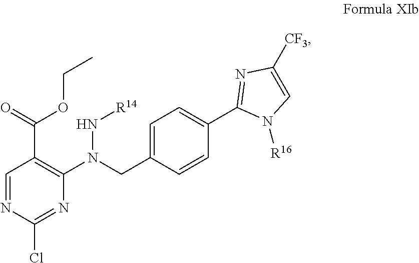

or a salt thereof, with (4-cyclopropyl-6-methoxypyrimidin-5-yl)boronic acid to form a compound of Formula VIIIb, wherein R¹⁴ is an amine protecting group.

In some embodiments, Formula IXb is Formula IX:

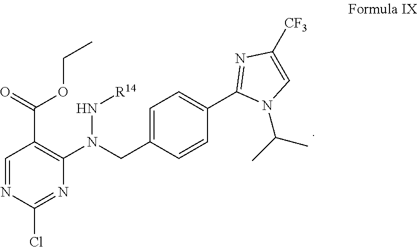

In some embodiments, said coupling occurs in the presence of a palladium catalyst.

In some embodiments, said coupling occurs in the presence of a solvent selected from 1,4-dioxane, water, and mixtures thereof.

In some embodiments, said coupling occurs at a temperature of from about 20° C. to about 65° C.

In another aspect, the present disclosure relates to a process for preparing a compound of Formula IXb:

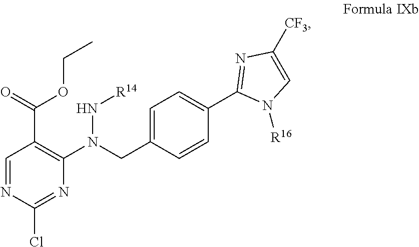

or a salt thereof, comprising reacting a compound of Formula Xb:

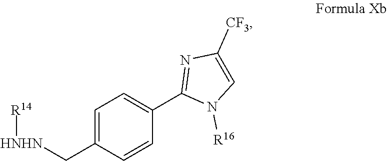

or a salt thereof, with a 2,4-dichloropyrimidine-5-carboxylate ester to form a compound of Formula IXb, wherein R¹⁴ is an amine protecting group.

In some embodiments, Formula Xb is Formula X:

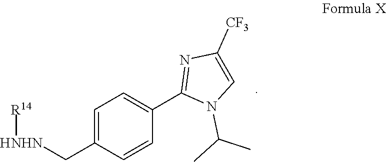

In some embodiments, the 2,4-dichloropyrimidine-5-carboxylate ester is ethyl 2,4-dichloropyrimidine-5-carboxylate.

In some embodiments, the 2,4-dichloropyrimidine-5-carboxylate ester is isopropyl 2,4-dichloropyrimidine-5-carboxylate.

In some embodiments, the compound of Formula X is reacted with the 2,4-dichloropyrimidine-5-carboxylate ester in the presence of a base and a solvent. In some embodiments, the base is an amine base, and the solvent is an alcohol solvent. In some embodiments, the amine base is 2,6-lutidine. In some embodiments, the solvent is isopropanol.

In some embodiments, the amine protecting group is t-butyloxycarbonyl (Boc).

In another aspect, the present disclosure relates to a process for preparing a compound of Formula Xb:

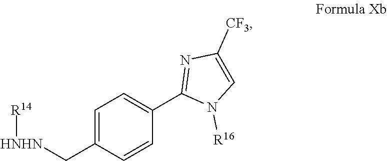

or a salt thereof, comprising reacting a compound of Formula XI:

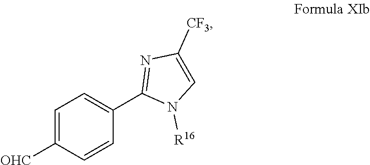

with tert-butyl carbazate and a reducing agent to form a compound of Formula Xb.

In some embodiments, Formula XIb is Formula XI:

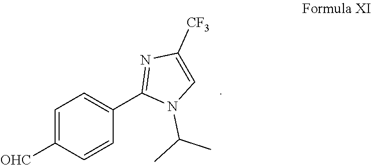 [SMILES: CC(C)n1cc(C(F)(F)F)nc1-c1ccc(C=O)cc1]

In some embodiments, the compound of Formula XI is reacted with tert-butyl carbazate in the presence of sodium cyanoborohydride.

In some embodiments, the compound of Formula XI is reacted with tert-butyl carbazate in the presence of hydrogen and a transition metal catalyst.

In another aspect, the present disclosure relates to a process for preparing a compound of Formula XIb:

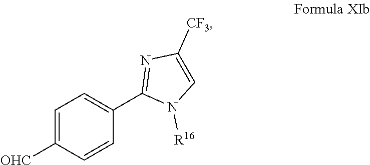

comprising reacting a compound of Formula XIIb:

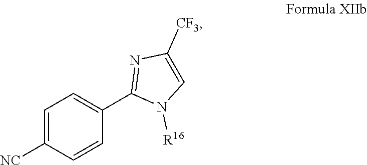

with a reducing agent to form a compound of Formula XIb.

In some embodiments, Formula XIIb is Formula XII:

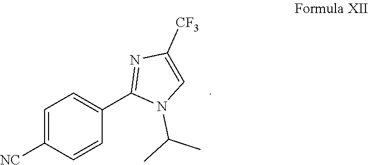 [SMILES: CC(C)n1cc(C(F)(F)F)nc1-c1ccc(C#N)cc1]

In some embodiments, the reducing agent is diisobutylaluminum hydride.

In another aspect, the present disclosure relates to a process for preparing a compound of Formula XII:

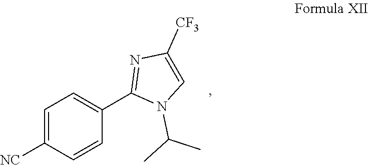 [SMILES: CC(C)n1cc(C(F)(F)F)nc1-c1ccc(C#N)cc1]

comprising reacting a compound of Formula XIII:

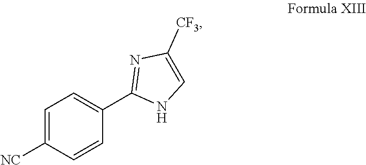 [SMILES: N#Cc1ccc(-c2nc(C(F)(F)F)c[nH]2)cc1]

with an alkylating agent to form a compound of Formula XII.

In some embodiments, the alkylating agent is selected from 2-iodopropane, 2-bromopropane, 2-isopropyl mesylate, and 2-isopropyl tosylate. In some embodiments, the alkylating agent is 2-iodopropane.

In another aspect, the present disclosure relates to a process for preparing a compound of Formula XIVb:

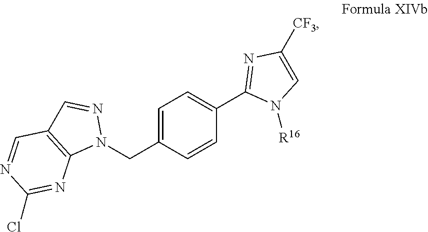

comprising reacting a compound of Formula XVb:

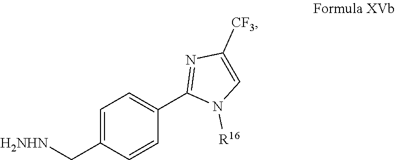

or a salt thereof, with a compound of Formula XVI:

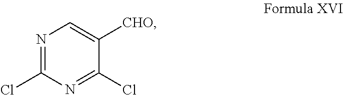

to form a compound of Formula XIVb;
wherein R¹⁶ is selected from hydrogen, alkyl, and an amine protecting group.

In some embodiments of the process, R¹⁶ is hydrogen. In some embodiments, R¹⁶ is an amine protecting group. In some embodiments, R¹⁶ is alkyl. In some embodiments, R¹⁶ is (C₁-C₅) alkyl. In some embodiments, R⁶ is methyl. In some embodiments, R¹⁶ is ethyl. In some embodiments, R¹⁶ is n-propyl. In some embodiments, R¹⁶ is isopropyl.

In some embodiments of the process, Formula XIVb is Formula XIV:

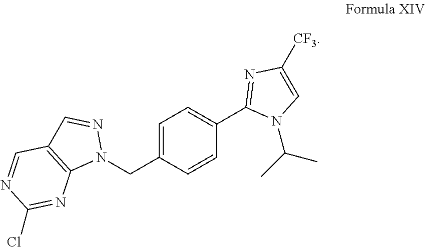 [SMILES: CC(C)n1cc(C(F)(F)F)nc1-c1ccc(Cn2ncc3cnc(Cl)nc32)cc1]

In some embodiments of the process, Formula XVb is Formula XV:

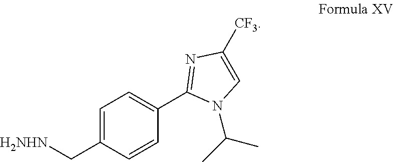

In some embodiments, the compound of Formula XV is reacted with the compound of Formula XVI in the presence of a solvent and a base. In some embodiments, the solvent is selected from ethanol, isopropanol, and tetrahydrofuran. In some embodiments, the base is an amine base. In some embodiments, the base is selected from triethylamine and diisopropylethylamine.

In some embodiments, the compound of Formula XV is reacted with the compound of Formula XVI at a temperature of from −20° C. to 25° C.

In some embodiments, the reaction of the compound of Formula XV with the compound of Formula XVI also forms a compound of Formula XVII:

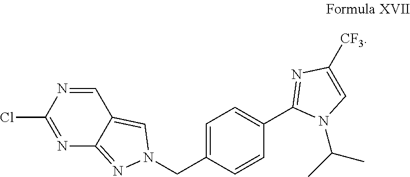 [SMILES: CC(C)n1cc(C(F)(F)F)nc1-c1ccc(Cn2cc3cnc(Cl)nc3n2)cc1]

In some embodiments, the compound of Formula XVII forms in an amount of less than 5% AUC as compared to the area of the compound of Formula XIV. In some embodiments, the compound of Formula XVII forms in an amount of less than 3% AUC as compared to the area of the compound of Formula XIV.

In some embodiments, the compound of Formula XIV is isolated containing less than 1%, less than 0.5%, less than 0.25%, or less than 0.15% AUC of the compound of Formula XVII. In some embodiments, the compound of Formula XIV is isolated containing less than 0.15% AUC of the compound of Formula XVII. In some embodiments, the compound of Formula XIV is isolated containing undetectable amounts AUC of the compound of Formula XVII.

In another aspect, the present disclosure relates to a process for preparing a compound of Formula XIVb:

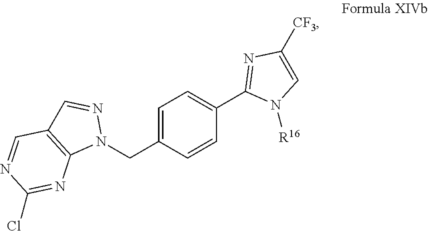

comprising reacting a compound of Formula Xb:

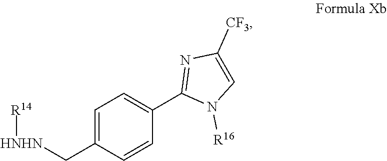

or a salt thereof, with a compound of Formula XVI:

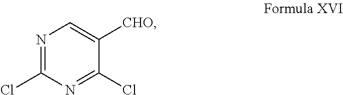

to form a compound of Formula XIVb.

In some embodiments, Formula Xb is Formula X:

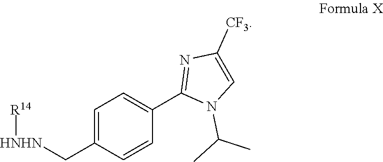

In some embodiments, Formula XIVb is Formula XIV:

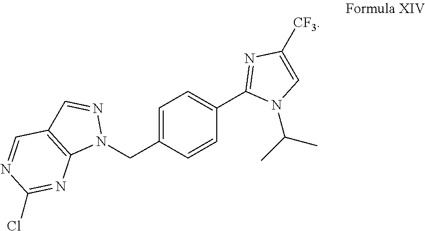 [SMILES: CC(C)n1cc(C(F)(F)F)nc1-c1ccc(Cn2ncc3cnc(Cl)nc32)cc1]

In another aspect, the present disclosure relates to a process comprising reacting the compound of Formula XIV with a compound of Formula XXI

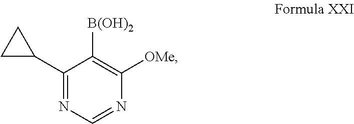 [SMILES: COc1ncnc(C2CC2)c1B(O)O]

to obtain a compound of Formula VI

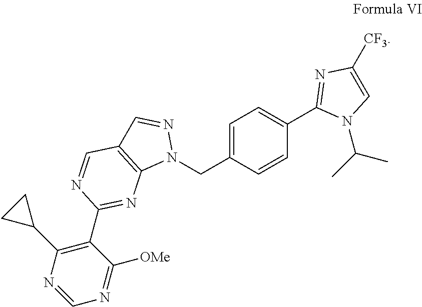 [SMILES: COc1ncnc(C2CC2)c1-c1ncc2cnn(Cc3ccc(-c4nc(C(F)(F)F)cn4C(C)C)cc3)c2n1]

In another aspect, the present disclosure relates to a process comprising mixing the compound of Formula VI with gentisic acid in a solvent to form a compound of Formula XVIII.

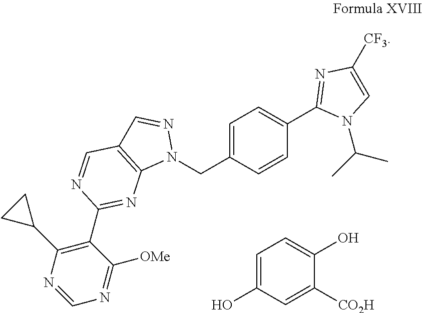 [SMILES: COc1ncnc(C2CC2)c1-c1ncc2cnn(Cc3ccc(-c4nc(C(F)(F)F)cn4C(C)C)cc3)c2n1 ; O=C(O)c1cc(O)ccc1O]

In some embodiments, the compound of Formula XVIII is a co-crystal.

In another aspect, the present disclosure relates to a process for preparing a compound of Formula XXX:

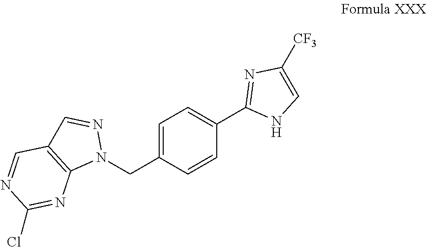 [SMILES: FC(F)(F)c1c[nH]c(-c2ccc(Cn3ncc4cnc(Cl)nc43)cc2)n1]

comprising:
reacting a compound of Formula XXVIV:

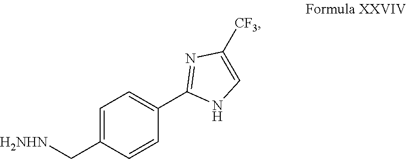

or a salt thereof,
with a compound of Formula XVI

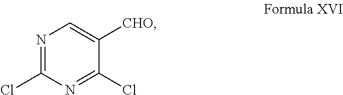

to form a compound of Formula XXX.

In some embodiments, the compound of Formula XXVIV is reacted with the compound of Formula XVI in the presence of a solvent and a base. In some embodiments, the solvent is selected from ethanol, isopropanol, or THF. In some embodiments, the base is an amine base. In some embodiments, the base is selected from triethylamine and diisopropylethylamine. In some embodiments, the reaction is conducted at a temperature of between −20° C. and 25° C.

In some embodiments, the process further comprises reacting the compound of Formula XXX with a compound of Formula XXI:

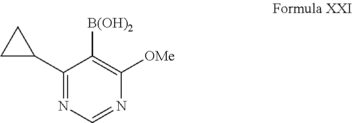 [SMILES: COc1ncnc(C2CC2)c1B(O)O]

to form a compound of Formula XXXI:

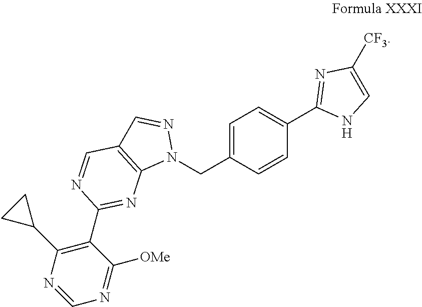 [SMILES: COc1ncnc(C2CC2)c1-c1ncc2cnn(Cc3ccc(-c4nc(C(F)(F)F)c[nH]4)cc3)c2n1]

In some embodiments, the process further comprises reacting the compound of Formula XXXI with

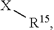

wherein X is a leaving group and R¹⁵ is an alkyl group;
to form a compound of Formula XXXII:

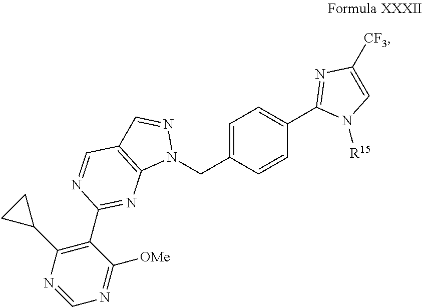

or a salt thereof

In some embodiments, R¹⁵ is an isopropyl group. In some embodiments, R¹⁵ is a methyl group.

In another aspect, the present disclosure relates to a process for preparing a compound of Formula XXVIV:

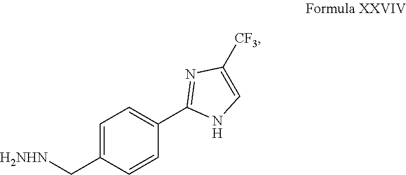

or a salt thereof,
comprising:
   reacting a compound of Formula XXVIII

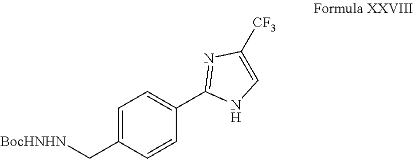

with acid to form a compound of Formula XXVIV or a salt thereof.

In some embodiments, the acid is selected from hydrochloric acid (HCl), hydrobromic acid (HBr), methanesulfonic acid, toluenesulfonic acid, trifluoroacetic acid, and trifluoromethanesulfonic acid. In some embodiments, the reaction is conducted at a temperature of between between 0 and 75° C.

In another aspect, the present disclosure relates to a process for preparing a compound of Formula XXVIII:

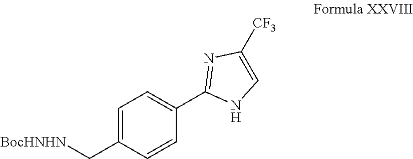

or a salt thereof,
comprising:
   reacting a compound of Formula XXVII:

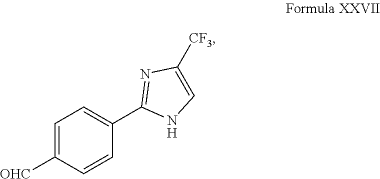 [SMILES: O=Cc1ccc(-c2nc(C(F)(F)F)c[nH]2)cc1]

with tert-butyl carbazate and a reducing agent to form a compound of Formula XXVIII.

In some embodiments, the compound of Formula XXVII is reacted with tert-butyl carbazate in the presence of hydrogen and a transition metal catalyst.

Additional embodiments and advantages of the disclosure will be set forth, in part, in the description that follows, and will flow from the description, or can be learned by practice of the disclosure. The embodiments and advantages of the disclosure will be realized and attained by means of the elements and combinations particularly pointed out in the appended claims.

It is to be understood that both the foregoing summary and the following detailed description are exemplary and explanatory only, and are not restrictive of the invention as claimed.

### DETAILED DESCRIPTION OF THE INVENTION

In one aspect, the present disclosure relates to a Compound of the Disclosure that is a compound, or a pharmaceutically acceptable salt or solvate thereof, having Formula I:

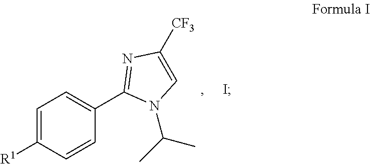

wherein:
   R¹ is selected from cyano, —CHO, —CH₂NR²R³, and —CH₂R⁴;
   R² is selected from hydrogen, optionally substituted aryl, and optionally substituted heteroaryl;
   R³ is selected from hydrogen, amino, and —NR⁵R⁶;
   R⁴ is selected from Formula II and Formula III:

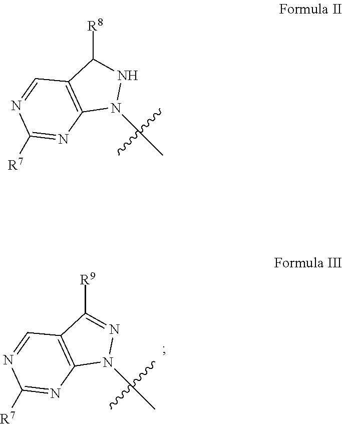

R⁵ and R⁶ are individually selected from hydrogen and an amine protecting group;
R⁷ is selected from optionally substituted aryl and optionally substituted heteroaryl;
R⁸ is selected from carbonyl, alkoxy, and sulfonate; and
R⁹ is selected from hydroxy, alkoxy, sulfonate, and a leaving group;

In some embodiments, a Compound of the Disclosure is a compound having Formula I, wherein R¹ is cyano. In some embodiments, R¹ is —CH₂NR²R³. In some embodiments, R¹ is —CH₂R⁴.

In some embodiments, a Compound of the Disclosure is a compound having Formula I, wherein R² is hydrogen. In some embodiments, R² is optionally substituted aryl. In some embodiments, R² is optionally substituted heteroaryl.

In some embodiments, a Compound of the Disclosure is a compound having Formula I, wherein R² is optionally substituted C₆ aryl. In some embodiments, R² is optionally substituted 6-membered heteroaryl.

In some embodiments, a Compound of the Disclosure is a compound having Formula I, wherein R² is optionally substituted phenyl. In some embodiments, R² is optionally substituted pyrimidinyl. In some embodiments, R² is optionally substituted pyridyl. In some embodiments, R² is optionally substituted pyrazinyl. In some embodiments, R² is optionally substituted pyridazinyl. In some embodiments, R² is optionally substituted pyrazolyl.

In some embodiments, a Compound of the Disclosure is a compound having Formula I, wherein the optional substituents on R² are independently selected from hydrogen, halo, hydroxy, alkyl, alkoxy, alkoxycarbonyl, cycloalkyl, optionally substituted aryl, and optionally substituted heteroaryl.

In another embodiment, the optional substituents on R² are independently selected from hydrogen, halo, hydroxy, (C₁-C₆) alkyl, (C₁-C₆) alkoxy, (C₁-C₄) alkoxycarbonyl, optionally substituted C₆ aryl, and optionally substituted 6-membered heteroaryl. In some embodiments, the optionally substituted C₆ aryl and optionally substituted 6-membered heteroaryl are substituted with alkoxy and cycloalkyl. In some embodiments, the optionally substituted C₆ aryl and optionally substituted 6-membered heteroaryl are substituted with methoxy and cyclopropyl.

In some embodiments, a Compound of the Disclosure is a compound having Formula I, wherein R³ is hydrogen. In some embodiments, a Compound of the Disclosure is a compound having Formula I, wherein R³ is amino. In some embodiments, a Compound of the Disclosure is a compound having Formula I, wherein R³ is —NR⁵R⁶.

In some embodiments, a Compound of the Disclosure is a compound having Formula I, wherein R¹ is —CH₂NR²R³, R² is hydrogen, and R³ is amino.

In some embodiments, a Compound of the Disclosure is a compound having Formula I, wherein R¹ is —CH₂NR²R³ and R² is an optionally substituted pyrimidinyl.

In some embodiments, a Compound of the Disclosure is a compound having Formula I, wherein R⁴ is Formula II:

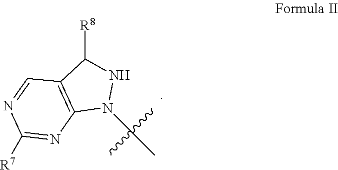

In some embodiments, a Compound of the Disclosure is a compound having Formula I, wherein R⁴ is Formula III:

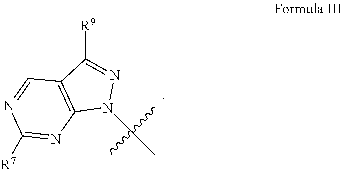

In some embodiments, a Compound of the Disclosure is a compound having Formula I, wherein R⁵ is hydrogen. In some embodiments, R⁵ is an amine protecting group. In some embodiments, the amine protecting group is selected from t-butyloxycarbonyl (Boc), fluorenylmethoxycarbonyl (Fmoc), benzyloxycarbonyl (Cbz), acetyl, trifluoroacetamide, phthalimide, benzyl, trityl, benzylideneamine, and toluenesulfonate. In some embodiments, the amine protecting group is t-butyloxycarbonyl (Boc). In some embodiments, the amine protecting group is fluorenylmethoxycarbonyl (Fmoc). In some embodiments, the amine protecting group is benzyloxycarbonyl (Cbz). In some embodiments, the amine protecting group is acetyl. In some embodiments, the amine protecting group is trifluoroacetamide. In some embodiments, the amine protecting group is phthalimide. In some embodiments, the amine protecting group is benzyl. In some embodiments, the amine protecting group is trityl. In some embodiments, the amine protecting group is benzylideneamine. In some embodiments, the amine protecting group is toluenesulfonate.

In some embodiments, a Compound of the Disclosure is a compound having Formula I, wherein R⁶ is hydrogen. In some embodiments, R⁶ is an amine protecting group. In some embodiments, the amine protecting group is selected from t-butyloxycarbonyl (Boc), fluorenylmethoxycarbonyl (Fmoc), benzyloxycarbonyl (Cbz), acetyl, trifluoroacetamide, phthalimide, benzyl, trityl, benzylideneamine, and toluenesulfonate. In some embodiments, the amine protecting group is t-butyloxycarbonyl (Boc). In some embodiments, the amine protecting group is fluorenylmethoxycarbonyl (Fmoc). In some embodiments, the amine protecting group is benzyloxycarbonyl (Cbz). In some embodiments, the amine protecting group is acetyl. In some embodiments, the amine protecting group is trifluoroacetamide. In some embodiments, the amine protecting group is phthalimide. In some embodiments, the amine protecting group is benzyl. In some embodiments, the amine protecting group is trityl. In some embodiments, the amine protecting group is benzylideneamine. In some embodiments, the amine protecting group is toluenesulfonate.

In some embodiments, a Compound of the Disclosure is a compound having Formula I, wherein one of R⁵ and R⁶ is hydrogen and the other of R⁵ and R⁶ is an amine protecting group selected from t-butyloxycarbonyl (Boc), fluorenylmethoxycarbonyl (Fmoc), benzyloxycarbonyl (Cbz), acetyl, trifluoroacetamide, phthalimide, benzyl, trityl, benzylideneamine, and toluenesulfonate. In some embodiments, the amine protecting group is t-butyloxycarbonyl (Boc).

In some embodiments, a Compound of the Disclosure is a compound having Formula I, wherein R⁵ and R⁶ are hydrogen. In some embodiments, R⁵ and R⁶ are amine protecting groups. In some embodiments, R⁵ is hydrogen and and R⁶ is an amine protecting group. In some embodiments, R⁶ is hydrogen and and R⁵ is an amine protecting group. In some embodiments, the amine protecting group is selected from t-butyloxycarbonyl (Boc), fluorenylmethoxycarbonyl (Fmoc), benzyloxycarbonyl (Cbz), acetyl, trifluoroacetamide, phthalimide, benzyl, trityl, benzylideneamine, and toluenesulfonate. In some embodiments, the amine protecting group is t-butyloxycarbonyl (Boc).

In some embodiments, a Compound of the Disclosure is a compound having Formula I, wherein R⁷ is optionally substituted aryl. In some embodiments, R⁷ is optionally substituted heteroaryl.

In some embodiments, a Compound of the Disclosure is a compound having Formula I, wherein R¹ is optionally substituted C₆ aryl. In some embodiments, R⁷ is optionally substituted 6-membered heteroaryl.

In some embodiments, a Compound of the Disclosure is a compound having Formula I, wherein the optional substituents on R⁷ are independently selected from hydrogen, halo, hydroxy, alkyl, alkoxy, and cycloalkyl.

In another embodiment, the optional substituents on R⁷ are independently selected from hydrogen, halo, hydroxy, (C₁-C₆) alkyl, (C₁-C₆) alkoxy, and (C₁-C₆) cycloalkyl.

In some embodiments, a Compound of the Disclosure is a compound having Formula I, wherein R⁷ has a Formula XIX:

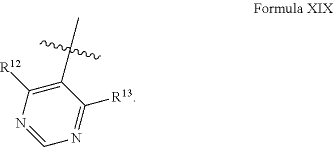

In some embodiments, a Compound of the Disclosure is a compound having Formula I, wherein R^{s} is a carbonyl. In some embodiments, R⁸ is alkoxy. In some embodiments, R⁸ is sulfonate. In some embodiments, R⁸ is (C₁-C₆) alkoxy.

In some embodiments, a Compound of the Disclosure is a compound having Formula I, wherein R⁹ is hydroxy. In some embodiments, R⁹ is alkoxy. In some embodiments, R⁹ is sulfonate. In some embodiments, R⁹ is a leaving group. In some embodiments, R⁹ is (C₁-C₆) alkoxy. A compound of Formula I, wherein R⁹ is hydroxy is a tautomer of a compound of formula I, wherein R⁸ is a carbonyl. Likewise, a compound of Formula I, wherein R^{s} is a carbonyl is a tautomer of a compound of formula I, wherein R⁹ is hydroxy.

In some embodiments, R⁹ is a leaving group selected from trifluoromethane-sulfonate, toluenesulfonate, methanesulfonate, and halogen. In some embodiments, the leaving group is trifluoromethanesulfonate. In some embodiments, the leaving group is toluenesulfonate. In some embodiments, the leaving group is methanesulfonate. In some embodiments, the leaving group is a halogen.

R⁹ hydroxy is tautomer with R⁸ as carbonyl.

In some embodiments, a Compound of the Disclosure is a compound having Formula I, wherein R¹² and R¹³ are individually selected from hydrogen, hydroxy, alkyl, alkoxy, and cycloalkyl.

In some embodiments, R¹² and R¹³ are individually selected from hydrogen, hydroxy, (C₁-C₆) alkyl, (C₁-C₆) alkoxy, and (C₁-C₆) cycloalkyl. In some embodiments, one of R¹² and R¹³ is cyclopropyl. In some embodiments, one of R¹² and R¹³ is methoxy. In some embodiments, one of R¹² and R¹³ is cyclopropyl and the other of R¹² and R¹³ is methoxy.

In some embodiments, a Compound of the Disclosure is a compound having Formula I, selected from the group consisting of:

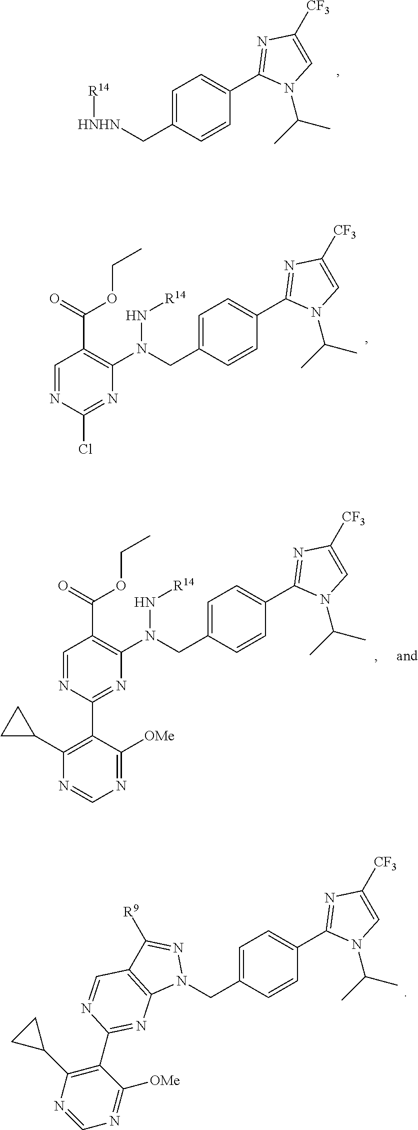

In some embodiments, a Compound of the Disclosure is a compound having Formula I, selected from the group consisting of:

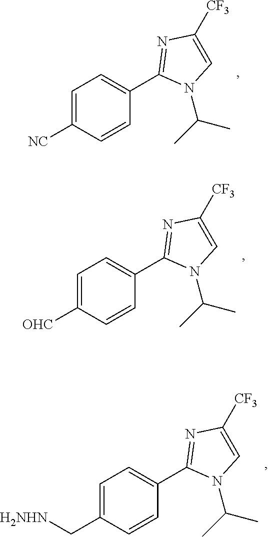

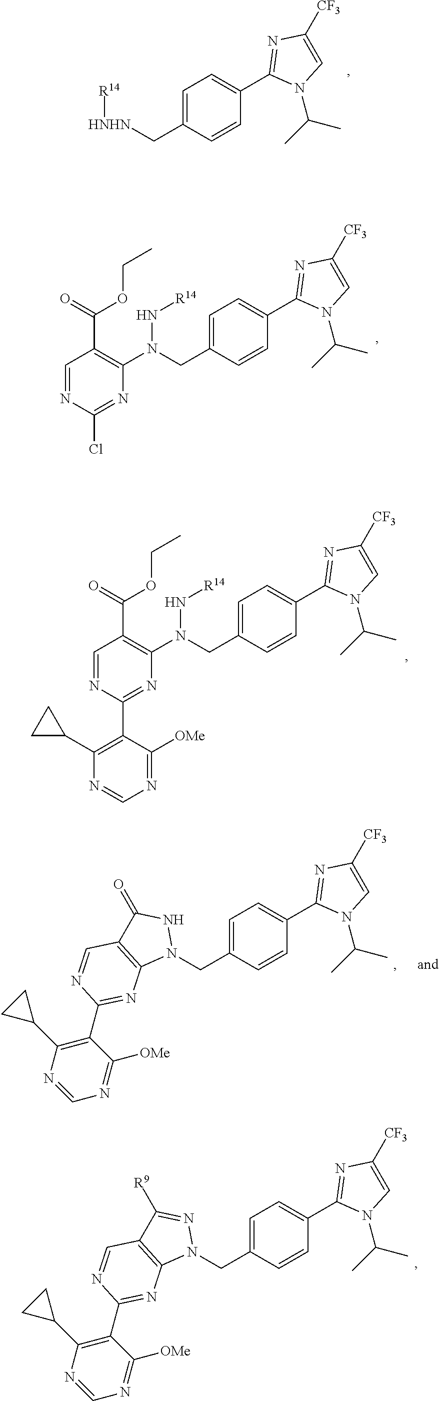

or a salt thereof.

In some embodiments, R⁹ is a leaving group. In some embodiments, the leaving group is selected from trifluoromethanesulfonate, toluenesulfonate, methanesulfonate, and halogen. In some embodiments, R⁹ is trifluoromethanesulfonate.

In some embodiments, R¹⁴ is an amine protecting group. In some embodiments, the amine protecting group is selected from t-butyloxycarbonyl (Boc), fluorenylmethoxycarbonyl (Fmoc), benzyloxycarbonyl (Cbz), acetyl, trifluoroacetamide, phthalimide, benzyl, trityl, benzylideneamine, and toluenesulfonate. In some embodiments, the amine protecting group is t-butyloxycarbonyl (Boc).

In some embodiments, a Compound of the Disclosure is a compound having Formula I, selected from the group consisting of:

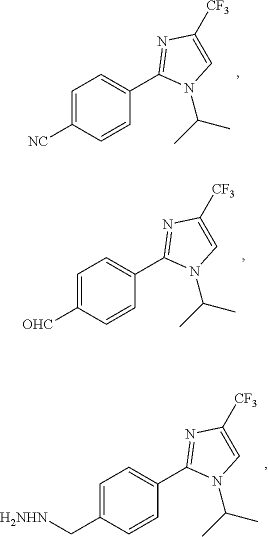

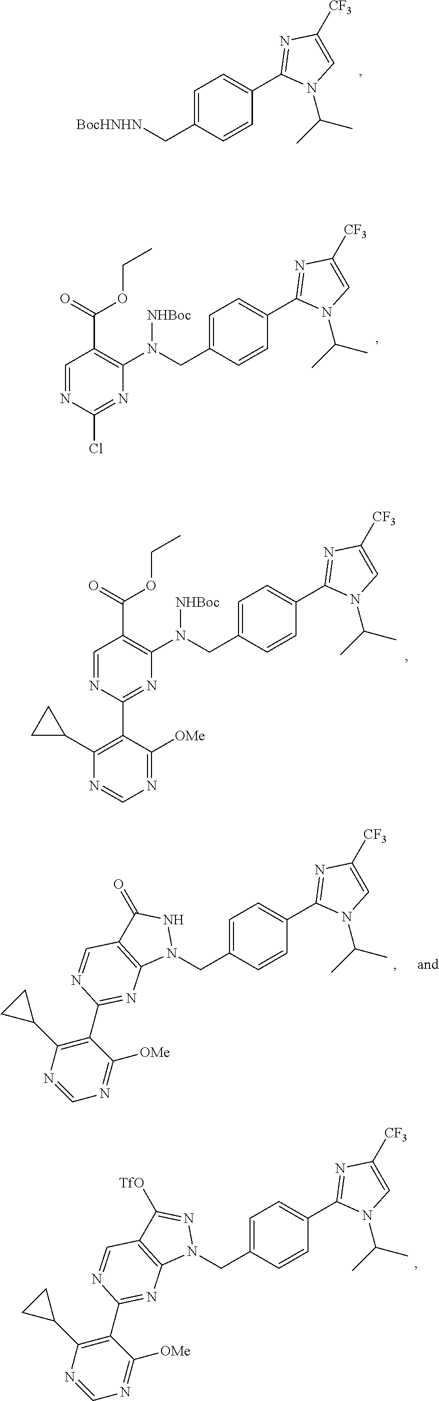

or a pharmaceutically acceptable salt thereof.

In another aspect, the present disclosure relates to a Compound of the Disclosure that is a compound, or a pharmaceutically acceptable salt or solvate thereof, having Formula IV:

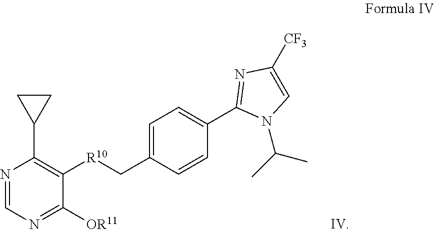

In some embodiments, a Compound of the Disclosure is a compound having Formula IV, wherein R¹⁰ is

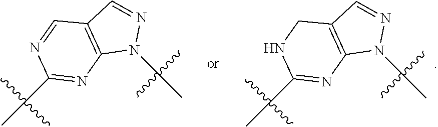 [SMILES: *c1ncc2cnn(*)c2n1 ; *C1=Nc2c(cnn2*)CN1]

In some embodiments, R¹⁰ is

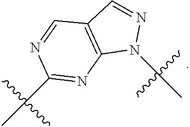 [SMILES: *c1ncc2cnn(*)c2n1]

In some embodiments, R¹⁰ is

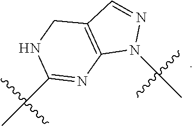 [SMILES: *C1=Nc2c(cnn2*)CN1]

In some embodiments, a Compound of the Disclosure is a compound having Formula IV, wherein R¹¹ is hydrogen or alkyl. In some embodiments, R¹¹ is hydrogen. In some embodiments, R¹¹ is alkyl. In some embodiments, R¹¹ is (C₁-C₆) alkyl. In some embodiments, R¹¹ is methyl.

In some embodiments, a Compound of the Disclosure is a compound having Formula IV, selected from the group consisting of:

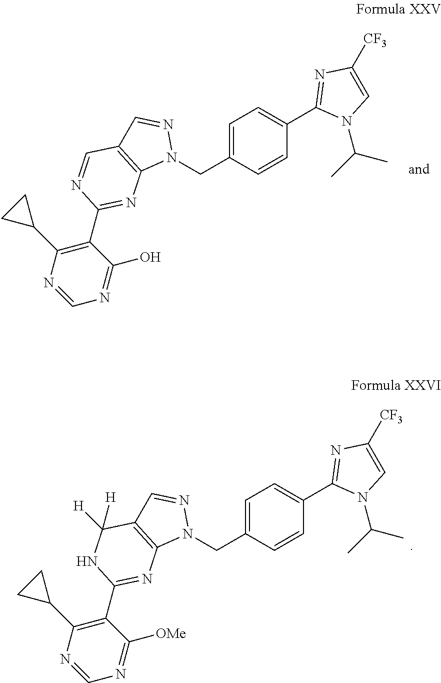 [SMILES: CC(C)n1cc(C(F)(F)F)nc1-c1ccc(Cn2ncc3cnc(-c4c(O)ncnc4C4CC4)nc32)cc1 ; COc1ncnc(C2CC2)c1C1=Nc2c(cnn2Cc2ccc(-c3nc(C(F)(F)F)cn3C(C)C)cc2)CN1]

In some embodiments, the compound is:

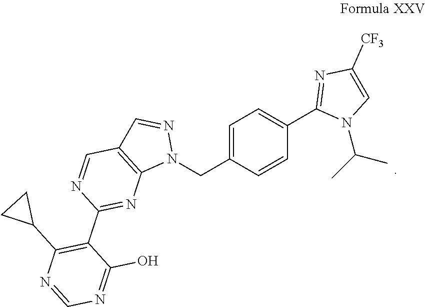 [SMILES: CC(C)n1cc(C(F)(F)F)nc1-c1ccc(Cn2ncc3cnc(-c4c(O)ncnc4C4CC4)nc32)cc1]

In some embodiments, the compound is:

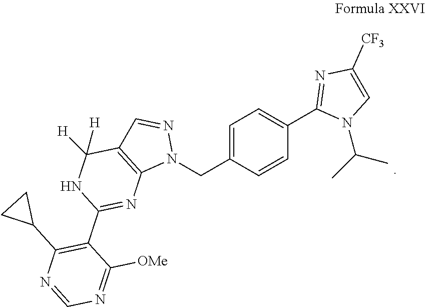 [SMILES: COc1ncnc(C2CC2)c1C1=Nc2c(cnn2Cc2ccc(-c3nc(C(F)(F)F)cn3C(C)C)cc2)CN1]

In another aspect, the present disclosure relates to a Compound of the Disclosure that is a compound, or a salt or solvate thereof, having Formula XIV:

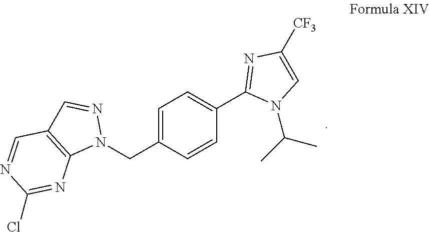 [SMILES: CC(C)n1cc(C(F)(F)F)nc1-c1ccc(Cn2ncc3cnc(Cl)nc32)cc1]

In another aspect, the present disclosure relates to a Compound of the Disclosure that is a compound, or a salt or solvate thereof, having Formula XVII:

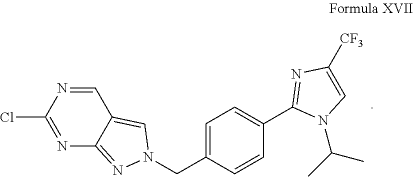 [SMILES: CC(C)n1cc(C(F)(F)F)nc1-c1ccc(Cn2cc3cnc(Cl)nc3n2)cc1]

In another aspect, the present disclosure relates to a Compound of the Disclosure that is a compound, or a salt or solvate thereof, having Formula XX:

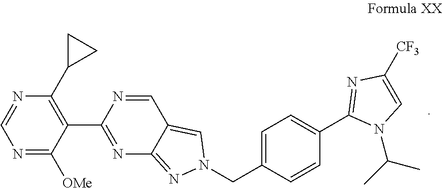 [SMILES: COc1ncnc(C2CC2)c1-c1ncc2cn(Cc3ccc(-c4nc(C(F)(F)F)cn4C(C)C)cc3)nc2n1]

In another aspect, the present disclosure relates to a Compound of the Disclosure that is a compound, or a salt or solvate thereof, having Formula XXVIII:

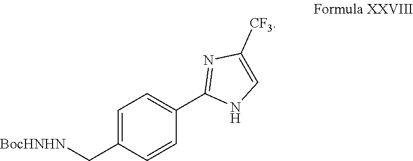

In another aspect, the present disclosure relates to a Compound of the Disclosure that is a compound, or a salt or solvate thereof, having Formula XXVIV:

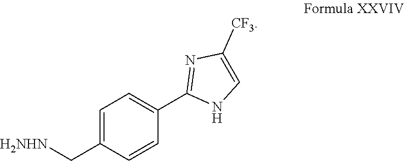

In another aspect, the present disclosure relates to a Compound of the Disclosure that is a compound, or a salt or solvate thereof, having Formula XXX:

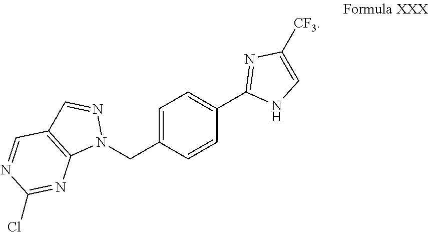 [SMILES: FC(F)(F)c1c[nH]c(-c2ccc(Cn3ncc4cnc(Cl)nc43)cc2)n1]

In another aspect, the present disclosure relates to a process for preparing a compound of Formula VIb:

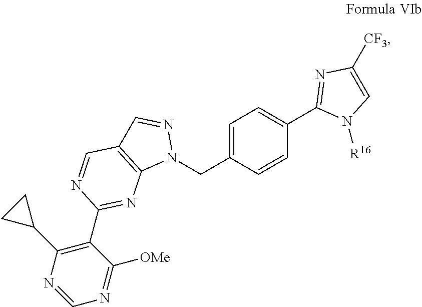

comprising reducing a compound of Formula Vb:

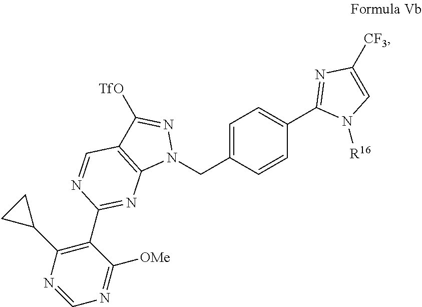

wherein R¹⁶ is selected from hydrogen, alkyl, and an amine protecting group.

In some embodiments of the process, R¹⁶ is hydrogen. In some embodiments, R¹⁶ is an amine protecting group. In some embodiments, R¹⁶ is alkyl. In some embodiments, R¹⁶ is (C₁-C₅) alkyl. In some embodiments, R¹⁶ is methyl. In some embodiments, R¹⁶ is ethyl. In some embodiments, R¹⁶ is n-propyl. In some embodiments, R¹⁶ is isopropyl.

In some embodiments of the process, Formula VIb is Formula VI:

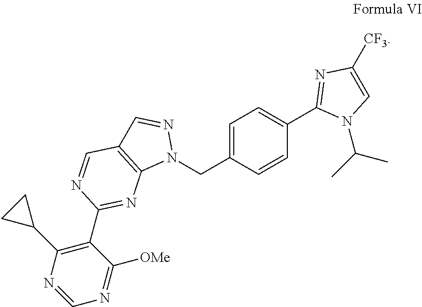 [SMILES: COc1ncnc(C2CC2)c1-c1ncc2cnn(Cc3ccc(-c4nc(C(F)(F)F)cn4C(C)C)cc3)c2n1]

In some embodiments of the process, Formula Vb is Formula V:

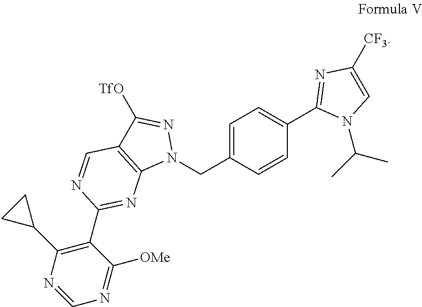 [SMILES: COc1ncnc(C2CC2)c1-c1ncc2c(OS(=O)(=O)C(F)(F)F)nn(Cc3ccc(-c4nc(C(F)(F)F)cn4C(C)C)cc3)c2n1]

In some embodiments, the process for preparing a compound of Formula VI comprises reducing a compound of Formula V in the presence of a palladium catalyst and a trialkylsilane or a dialkylsilane. In some embodiments, the palladium catalyst is (dppf)PdCl₂. In some embodiments, the palladium catalyst is (dppf)Pd(OAc)₂. In some embodiments, the palladium catalyst is Pd(PPh₃)₄. In some embodiments, the palladium catalyst is (dba)₃Pd₂ with XPhos. In some embodiments, the trialkylsilane or dialkylsilane is selected from triethylsilane, trimethylsilane, and diethylsilane. In some embodiments, the trialkylsilane is triethylsilane.

In some embodiments, the process for preparing a compound of Formula VI comprises reducing a compound of Formula V in the presence of a palladium catalyst and an ammonium compound. In some embodiments, the palladium catalyst is Pd/C and the ammonium compound is ammonium formate.

In some embodiments, the process for preparing a compound of Formula VI comprises reducing a compound of Formula V in the presence of a palladium catalyst and triethylsilane. In some embodiments, the process for preparing a compound of Formula VI comprises reducing a compound of Formula V in the presence of (dppf)PdCl₂ and triethylsilane.

In some embodiments, the process for preparing a compound of Formula VI comprises reducing a compound of Formula V in the presence of a palladium catalyst, a trialkylsilane, and a base. In some embodiments, the palladium catalyst is (dppf)PdCl₂. In some embodiments, the trialkylsilane is selected from triethylsilane and trimethylsilane. In some embodiments, the trialkylsilane is triethylsilane. In some embodiments, the base is an amine base. In some embodiments, the amine base is selected from, but is not limited to, triethylamine, tributylamine, 2,6-lutidine, N,N-diisopropylethylamine, 1,8-diazabicyclo[5.4.0]undec-7-ene (DBU), and 1,5-diazabicyclo[4.3.0]non-5-ene (DBN). In some embodiments, the amine base is a tertiary amine base, such as triethylamine or N,N-diisopropylethylamine. In some embodiments, the amine base is triethylamine.

In another aspect, the present disclosure relates to a process comprising mixing the compound of Formula VI with gentisic acid in a solvent to form a compound of Formula XVIII.

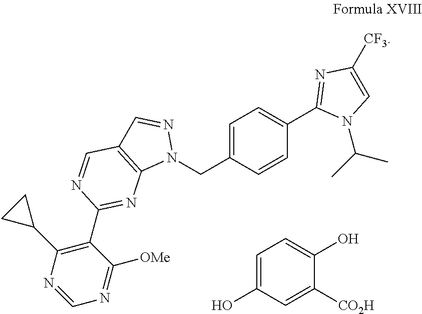 [SMILES: COc1ncnc(C2CC2)c1-c1ncc2cnn(Cc3ccc(-c4nc(C(F)(F)F)cn4C(C)C)cc3)c2n1 ; O=C(O)c1cc(O)ccc1O]

In some embodiments, the compound of Formula XVIII is a co-crystal.

In some embodiments, the compound of Formula VI is mixed with gentisic acid in the presence of a solvent. In some embodiments, the solvent is selected from, but is not limited to, ethyl acetate, heptane, acetone, isopropanol, and combinations thereof. In some embodiments, the solvent is isopropanol. In some embodiments, the solvent is heptane. In some embodiments, the solvent is a mixture of isopropanol and heptane.

In some embodiments, the compound of Formula XVIII is isolated from the solvent. In some embodiments, the compound of Formula XVIII is isolated from the solvent by precipitation. In some embodiments, the compound of Formula XVIII is isolated from the solvent by evaporation. In some embodiments, the compound of Formula XVIII is isolated from the solvent by crystalization. In some embodiments, the compound of Formula XVIII is re-crystallized using one or more solvents.

In some embodiments, the compound of Formula VI can be mixed with gentisic acid using the methods disclosed in U.S. Appl. Publication No. 2022/0162213, the disclosure of which is incorporated by reference.

In another aspect, the present disclosure relates to a process for preparing a compound of Formula Vab:

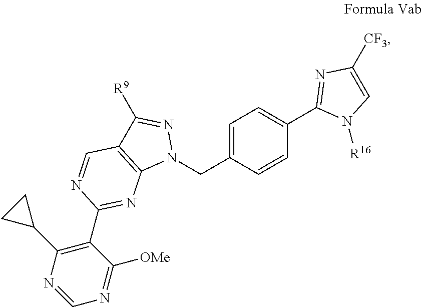

wherein R⁹ is a leaving group selected from trifluoromethanesulfonate, toluenesulfonate, methanesulfonate;
comprising reacting a compound of Formula VIIb:

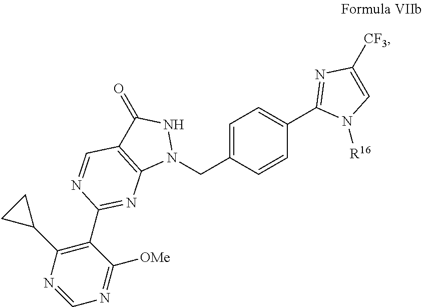

with a sulfonating agent to form a compound of Formula Vab.

In some embodiments, Formula Vab is Formula Va:

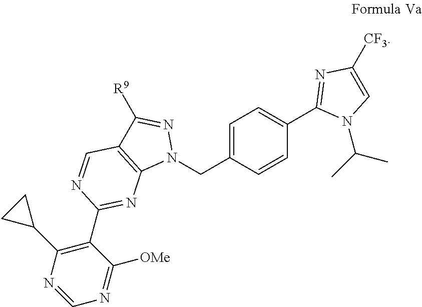

In some embodiments, Formula VIIb is Formula VII:

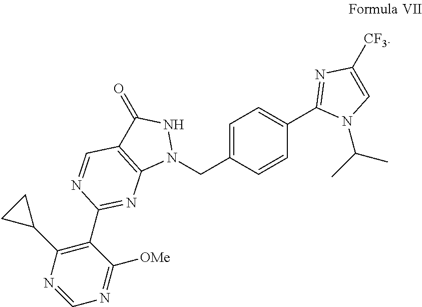 [SMILES: COc1ncnc(C2CC2)c1-c1ncc2c(=O)[nH]n(Cc3ccc(-c4nc(C(F)(F)F)cn4C(C)C)cc3)c2n1]

In some embodiments, the sulfonating agent is selected from toluenesulfonic anhydride, toluenesulfonyl chloride, methansulfonic anhydride, methansulfonyl chloride, trifluoromethanesulfonic anhydride, or trifluoromethanesulfonyl chloride. In some embodiments, the sulfonating agent is toluenesulfonic anhydride. In some embodiments, the sulfonating agent is toluenesulfonyl chloride. In some embodiments, the sulfonating agent is methansulfonic anhydride. In some embodiments, the sulfonating agent is methansulfonyl chloride. In some embodiments, the sulfonating agent is trifluoromethanesulfonic anhydride. In some embodiments, the sulfonating agent is trifluoromethanesulfonyl chloride.

In some embodiments, the compound of Formula VII is reacted with trifluoromethanesulfonic anhydride or trifluoromethanesulfonyl chloride to form a compound of Formula V. In some embodiments, the compound of Formula VII is reacted with trifluoromethanesulfonic anhydride to form a compound of Formula V. In some embodiments, the compound of Formula VII is reacted with trifluoromethanesulfonyl chloride to form a compound of Formula V.

In some embodiments, the compound of Formula VII is reacted with a sulfonating agent and a base to form a compound of Formula Va. In some embodiments, the base is an amine base. In some embodiments, the amine base is selected from, but not limited to, triethylamine, tributylamine, 2,6-lutidine, N,N-diisopropylethylamine, 1,8-diazabicyclo[5.4.0]undec-7-ene (DBU), and 1,5-diazabicyclo[4.3.0]non-5-ene (DBN).

In another aspect, the present disclosure relates to a process for preparing a compound of Formula VIIb:

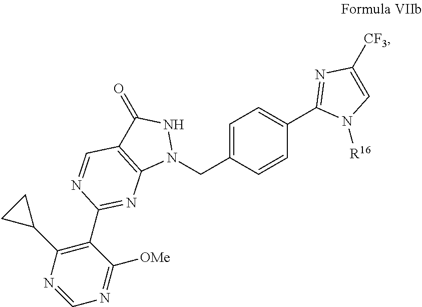

comprising reacting a compound of Formula VIIIb:

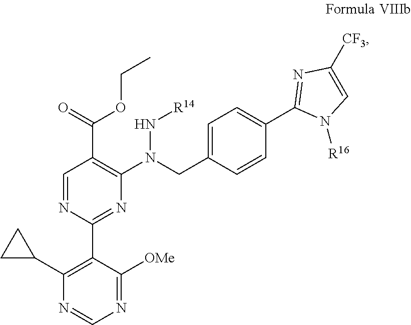

or a salt thereof, with an acid to form a compound of Formula VIIb. In some embodiments, the acid is selected from, but is not limited to, trifluoroacetic acid, hydrochloric acid, methanesulfonic acid, and trifluoromethanesulfonic acid. In some embodiments, the acid is trifluoroacetic acid.

In some embodiments, Formula VIIIb is Formula VIII:

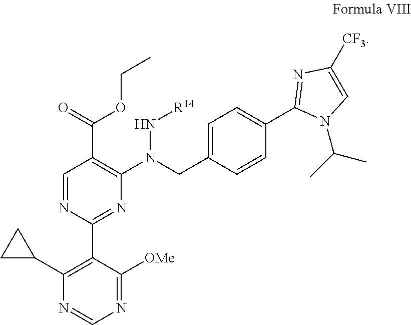

In some embodiments, R¹⁴ is an amine protecting group. In some embodiments, the amine protecting group is selected from t-butyloxycarbonyl (Boc), fluorenylmethoxycarbonyl (Fmoc), benzyloxycarbonyl (Cbz), acetyl, trifluoroacetamide, phthalimide, benzyl, trityl, benzylideneamine, and toluenesulfonate. In some embodiments, the amine protecting group is t-butyloxycarbonyl (Boc). In some embodiments, the amine protecting group is fluorenylmethoxycarbonyl (Fmoc). In some embodiments, the amine protecting group is benzyloxycarbonyl (Cbz). In some embodiments, the amine protecting group is acetyl. In some embodiments, the amine protecting group is trifluoroacetamide. In some embodiments, the amine protecting group is phthalimide. In some embodiments, the amine protecting group is benzyl. In some embodiments, the amine protecting group is trityl. In some embodiments, the amine protecting group is benzylideneamine. In some embodiments, the amine protecting group is toluenesulfonate.

In some embodiments, the compound of Formula VIII is a compound of Formula VIIIa:

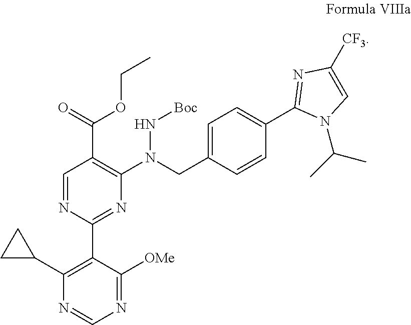 [SMILES: CCOC(=O)c1cnc(-c2c(OC)ncnc2C2CC2)nc1N(Cc1ccc(-c2nc(C(F)(F)F)cn2C(C)C)cc1)NC(=O)OC(C)(C)C]

In some embodiments, a compound of Formula VIII, or a salt thereof, is reacted with an acid in the presence of a solvent to form a compound of Formula VII. In some embodiments, the solvent is selected from, but is not limited to, toluene, xylene, dimethylsulfoxide (DMSO), acetonitrile, dioxane, N-Methylpyrrolidone (NMP), and dichloromethane (DCM). In some embodiments, the solvent is toluene. In some embodiments, the solvent is xylene. In some embodiments, the solvent is DMSO. In some embodiments, the solvent is acetonitrile. In some embodiments, the solvent is dioxane. In some embodiments, the solvent is NMP. In some embodiments, the solvent is DCM.

In some embodiments, the compound of Formula VII or VIIb is isolated from the solvent. In some embodiments, the compound of Formula VII or VIIb is isolated from the solvent by precipitation. In some embodiments, the compound of Formula VII or VIIb is isolated from the solvent by evaporation. In some embodiments, the compound of Formula VII or VIIb is isolated from the solvent by crystalization.

In some embodiments, a compound of Formula VIII, or a salt thereof, is reacted with an acid at a temperature of from about 0° C. to about 100° C. to form a compound of Formula VII. In some embodiments, the temperature is from about 10° C. to about 75° C. In some embodiments, the temperature is from about 20° C. to about 55° C. In some embodiments, the temperature is from about 25° C. to about 50° C. In some embodiments, the temperature is from about 30° C. to about 45° C. In some embodiments, the temperature is about 0° C., about 10° C., about 20° C., about 30° C., about 40° C., about 50° C., about 60° C., about 70° C., about 80° C., about 90° C., about 100° C., or any range of temperature values thereof.

In another aspect, the present disclosure relates to a process for preparing a compound of Formula VIIIb:

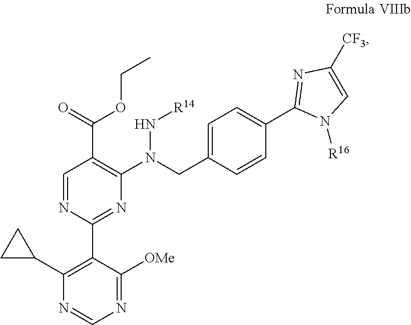

or a salt thereof, comprising coupling a compound of Formula IXb:

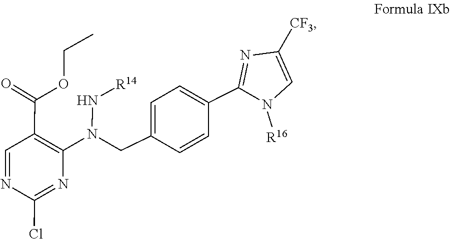

or a salt thereof, with (4-cyclopropyl-6-methoxypyrimidin-5-yl)boronic acid to form a compound of Formula VIIIb.

In some embodiments, Formula IXb is Formula IX:

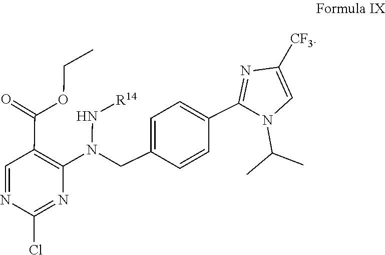

In some embodiments, R¹⁴ is an amine protecting group. In some embodiments, the amine protecting group is selected from t-butyloxycarbonyl (Boc), fluorenylmethoxycarbonyl (Fmoc), benzyloxycarbonyl (Cbz), acetyl, trifluoroacetamide, phthalimide, benzyl, trityl, benzylideneamine, and toluenesulfonate. In some embodiments, the amine protecting group is t-butyloxycarbonyl (Boc). In some embodiments, the amine protecting group is fluorenylmethoxycarbonyl (Fmoc). In some embodiments, the amine protecting group is benzyloxycarbonyl (Cbz). In some embodiments, the amine protecting group is acetyl. In some embodiments, the amine protecting group is trifluoroacetamide. In some embodiments, the amine protecting group is phthalimide. In some embodiments, the amine protecting group is benzyl. In some embodiments, the amine protecting group is trityl. In some embodiments, the amine protecting group is benzylideneamine. In some embodiments, the amine protecting group is toluenesulfonate.

In some embodiments, the compound of Formula VIII is a compound of Formula VIIIa:

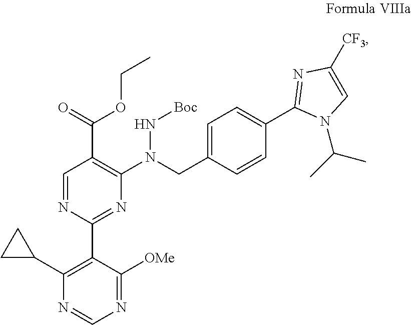 [SMILES: CCOC(=O)c1cnc(-c2c(OC)ncnc2C2CC2)nc1N(Cc1ccc(-c2nc(C(F)(F)F)cn2C(C)C)cc1)NC(=O)OC(C)(C)C]

and the compound of Formula IX is a compound of Formula IXa:

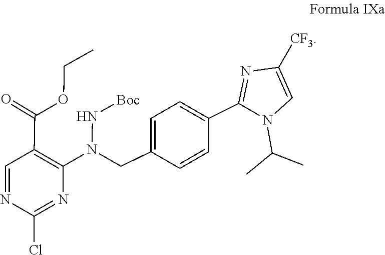 [SMILES: CCOC(=O)c1cnc(Cl)nc1N(Cc1ccc(-c2nc(C(F)(F)F)cn2C(C)C)cc1)NC(=O)OC(C)(C)C]

In some embodiments, a compound of Formula IX is coupled with with (4-cyclopropyl-6-methoxypyrimidin-5-yl)boronic acid in the presence of a palladium catalyst to form a compound of Formula VIII. In some embodiments, the palladium catalyst is (dppf)PdCl₂.

In some embodiments, a compound of Formula IX is coupled with with (4-cyclopropyl-6-methoxypyrimidin-5-yl)boronic acid in the presence of a solvent to form a compound of Formula VIII. In some embodiments, the solvent is selected from, but not limited to, 1,4-dioxane, water, tetrahydrofuran (TIF), 2-methyl-tetrahydrofuran, and mixtures thereof. In some embodiments, the solvent is 1,4-dioxane. In some embodiments, the solvent is water. In some embodiments, the solvent is THF. In some embodiments, the solvent is 2-methyl-tetrahydrofuran. In some embodiments, the solvent is a mixture of 1,4-dioxane and water.

In some embodiments, a compound of Formula IX is coupled with with (4-cyclopropyl-6-methoxypyrimidin-5-yl)boronic acid at a temperature of from about 0° C. to about 100° C. to form a compound of Formula VIII. In some embodiments, the temperature is from about 10° C. to about 80° C. In some embodiments, the temperature is from about 20° C. to about 65° C. In some embodiments, the temperature is from about 20° C. to about 55° C. In some embodiments, the temperature is from about 25° C. to about 50° C. In some embodiments, the temperature is from about 30° C. to about 45° C. In some embodiments, the temperature is about 0° C., about 10° C., about 20° C., about 30° C., about 40° C., about 50° C., about 60° C., about 70° C., about 80° C., about 90° C., about 100° C., or any range of temperature values thereof.

In some embodiments, a compound of Formula IX is coupled with with (4-cyclopropyl-6-methoxypyrimidin-5-yl)boronic acid in the presence of a base to form a compound of Formula VIII. In some embodiments, the base is selected from sodium carbonate, potassium carbonate, and cesium carbonate. In some embodiments, the base is cesium carbonate.

In another aspect, the present disclosure relates to a process for preparing a compound of Formula IXb:

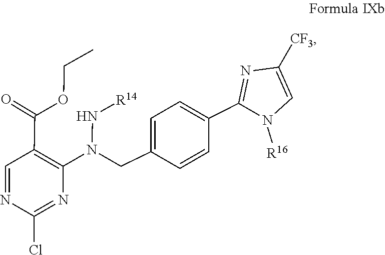

or a salt thereof, comprising reacting a compound of Formula Xb:

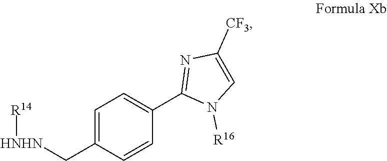

or a salt thereof, with a 2,4-dichloropyrimidine-5-carboxylate ester to form a compound of Formula IXb.

In some embodiments, Formula Xb is Formula X:

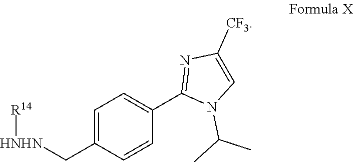

In some embodiments, the 2,4-dichloropyrimidine-5-carboxylate ester is selected from, but is not limited to, methyl 2,4-dichloropyrimidine-5-carboxylate, ethyl 2,4-dichloropyrimidine-5-carboxylate, and isopropyl 2,4-dichloropyrimidine-5-carboxylate. In some embodiments, the 2,4-dichloropyrimidine-5-carboxylate ester is methyl 2,4-dichloropyrimidine-5-carboxylate. In some embodiments, the 2,4-dichloropyrimidine-5-carboxylate ester is ethyl 2,4-dichloropyrimidine-5-carboxylate. In some embodiments, the 2,4-dichloropyrimidine-5-carboxylate ester is isopropyl 2,4-dichloropyrimidine-5-carboxylate.

In some embodiments, R¹⁴ is an amine protecting group. In some embodiments, the amine protecting group is selected from t-butyloxycarbonyl (Boc), fluorenylmethoxycarbonyl (Fmoc), benzyloxycarbonyl (Cbz), acetyl, trifluoroacetamide, phthalimide, benzyl, trityl, benzylideneamine, and toluenesulfonate. In some embodiments, the amine protecting group is t-butyloxycarbonyl (Boc). In some embodiments, the amine protecting group is fluorenylmethoxycarbonyl (Fmoc). In some embodiments, the amine protecting group is benzyloxycarbonyl (Cbz). In some embodiments, the amine protecting group is acetyl. In some embodiments, the amine protecting group is trifluoroacetamide. In some embodiments, the amine protecting group is phthalimide. In some embodiments, the amine protecting group is benzyl. In some embodiments, the amine protecting group is trityl. In some embodiments, the amine protecting group is benzylideneamine. In some embodiments, the amine protecting group is toluenesulfonate.

In some embodiments, a compound of Formula X is reacted with the 2,4-dichloropyrimidine-5-carboxylate ester in the presence of a base and a solvent to form a compound of Formula IX.

In some embodiments, the base is an amine base. The amine base is selected from, but is not limited to, triethylamine, tributylamine, 2,6-lutidine, N,N-diisopropylethylamine, 1,8-diazabicyclo[5.4.0]undec-7-ene (DBU), 1,5-diazabicyclo[4.3.0]non-5-ene (DBN), and 1,4-diazabicyclo[2.2.2]octane (DABCO). In some embodiments, the amine base is 2,6-lutidine.

In some embodiments, the solvent is an alcohol solvent. In some embodiments, the alcohol solvent is selected from, but is not limited to, ethanol, n-propanol, isopropanol, n-butanol, isobutanol, and tert-butanol. In some embodiments, the solvent is isopropanol.

In some embodiments, the solvent is an organic solvent. In some embodiments, the organic solvent is selected from, but is not limited to, toluene, methyl tert-butyl ether (MTBE), acetonitrile, and THF.

In another aspect, the present disclosure relates to a process for preparing a compound of Formula Xb:

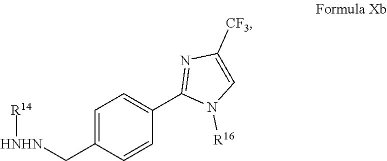

or a salt thereof, comprising reacting a compound of Formula XIb:

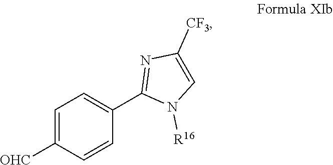

with tert-butyl carbazate and a reducing agent to form a compound of Formula Xb.

In some embodiments, Formula XIb is Formula XI:

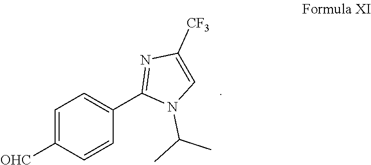 [SMILES: CC(C)n1cc(C(F)(F)F)nc1-c1ccc(C=O)cc1]

In some embodiments, a compound of Formula XI is reacted with tert-butyl carbazate in the presence of a reducing agent selected from, but not limited to, sodium cyanoborohydride, sodium triacetoxyborohydride, diisobutylaluminum hydride, lithium aluminum hydride, sodium borohydride, and sodium bis(2-methoxyethoxy)aluminium hydride to form a compound of Formula X. In some embodiments, the reducing agent is sodium cyanoborohydride.

In some embodiments, the compound of Formula XI is reacted with tert-butyl carbazate in the presence of hydrogen or sodium formate and a transition metal catalyst to form a compound of Formula X. In some embodiments, the transition metal catalyst is a palladium catalyst. In some embodiments, the palladium catalyst is palladium on carbon (Pd/C).

In another aspect, the present disclosure relates to a process for preparing a compound of Formula XIb:

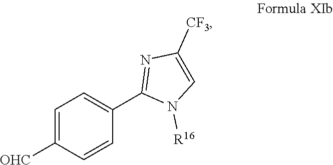

comprising reacting a compound of Formula XIIb:

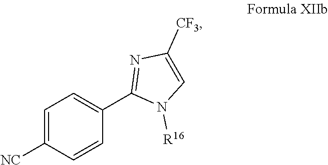

with a reducing agent to form a compound of Formula XI.

In some embodiments, Formula XIIb is Formula XII:

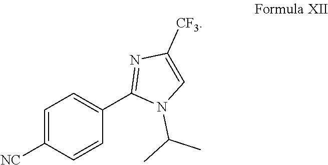 [SMILES: CC(C)n1cc(C(F)(F)F)nc1-c1ccc(C#N)cc1]

In some embodiments, a compound of Formula XII is reacted with a reducing agent selected from, but not limited to, diisobutylaluminum hydride and lithium aluminum hydride to form a compound of Formula XI. In some embodiments, the reducing agent is diisobutylaluminum hydride.

In another aspect, the present disclosure relates to a process for preparing a compound of Formula XII:

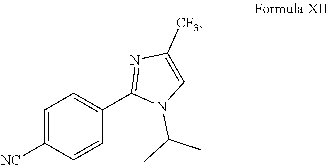 [SMILES: CC(C)n1cc(C(F)(F)F)nc1-c1ccc(C#N)cc1]

comprising reacting a compound of Formula XIII:

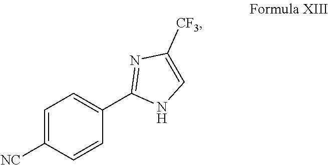 [SMILES: N#Cc1ccc(-c2nc(C(F)(F)F)c[nH]2)cc1]

with an alkylating agent to form a compound of Formula XII.

In some embodiments, the alkylating agent is selected from 2-iodopropane, 2-bromopropane, 2-chloropropane, 2-isopropyl mesylate, and 2-isopropyl tosylate. In some embodiments, the alkylating agent is 2-iodopropane. In some embodiments, the alkylating agent is 2-bromopropane. In some embodiments, the alkylating agent is 2-chloropropane. In some embodiments, the alkylating agent is 2-isopropyl mesylate. In some embodiments, the alkylating agent is 2-isopropyl tosylate.

In another aspect, the present disclosure relates to a process for preparing a compound of Formula XIVb:

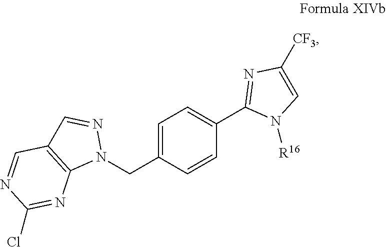

comprising reacting a compound of Formula XVb:

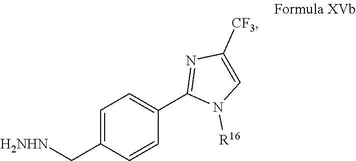

or a salt thereof, with a compound of Formula XVI:

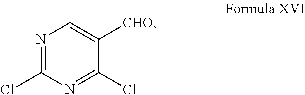

to form a compound of Formula XIVb;
wherein R¹⁶ is selected from hydrogen, alkyl, and an amine protecting group.

In some embodiments of the process, R¹⁶ is hydrogen. In some embodiments, R¹⁶ is an amine protecting group. In some embodiments, R¹⁶ is alkyl. In some embodiments, R¹⁶ is (C₁-C₅) alkyl. In some embodiments, R¹⁶ is methyl. In some embodiments, R¹⁶ is ethyl. In some embodiments, R¹⁶ is n-propyl. In some embodiments, R¹⁶ is isopropyl.

In some embodiments of the process, Formula XIVb is Formula XIV:

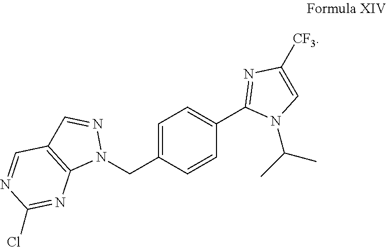 [SMILES: CC(C)n1cc(C(F)(F)F)nc1-c1ccc(Cn2ncc3cnc(Cl)nc32)cc1]

In some embodiments of the process, Formula XVb is Formula XV:

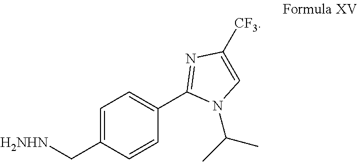

In some embodiments, the compound of Formula XV is reacted with the compound of Formula XVI in the presence of a solvent and a base to form a compound of Formula XIV. In some embodiments, the solvent is selected from, but is not limited to, methanol, ethanol, isopropanol, and tetrahydrofuran. In some embodiments, the solvent is methanol. In some embodiments, the solvent is ethanol. In some embodiments, the solvent is isopropanol. In some embodiments, the solvent is tetrahydrofuran.

In some embodiments, the base is an amine base. In some embodiments, the amine base is triethylamine, tributylamine, 2,6-lutidine, N,N-diisopropylethylamine, 1,8-diazabicyclo[5.4.0]undec-7-ene (DBU), and 1,5-diazabicyclo[4.3.0]non-5-ene (DBN). In some embodiments, the base is triethylamine. In some embodiments, the base is N,N-diisopropylethylamine.

In some embodiments, a compound of Formula XV is reacted with a compound of Formula XVI at a temperature of about −50° C. to about 50° C. to form a compound of Formula XIV. In some embodiments, the temperature is from about −30° C. to about 30° C. In some embodiments, the temperature is from about −20° C. to about 25° C. In some embodiments, the temperature is about −50° C., about −40° C., about −30° C., about −20° C., about −10° C., about 0° C., about 10° C., about 20° C., about 30° C., about 40° C., about 50° C., or any range of temperature values thereof.

In some embodiments, a reaction of a compound of Formula XV with a compound of Formula XVI also forms a compound of Formula XVII.

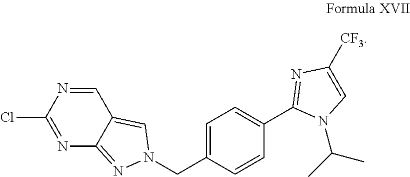 [SMILES: CC(C)n1cc(C(F)(F)F)nc1-c1ccc(Cn2cc3cnc(Cl)nc3n2)cc1]

In some embodiments, the compound of Formula XVII forms in an amount of less than 10% AUC as compared to the area of the compound of Formula XIV. In some embodiments, the compound of Formula XVII forms in an amount of less than 10%, or less than 9%, or less than 8%, or less than 7%, or less than 6%, or less than 5%, or less than 4%, or less than 3%, or less than 2%, or less than 1%, AUC as compared to the area of the compound of Formula XIV. In some embodiments, the compound of Formula XVII forms in an amount of less than 5% AUC as compared to the area of the compound of Formula XIV. In some embodiments, the compound of Formula XVII forms in an amount of less than 3% AUC as compared to the area of the compound of Formula XIV. In some embodiments, the compound of Formula XVII forms in an amount of less than 1% AUC as compared to the area of the compound of Formula XIV.

In some embodiments, the compound of Formula XVII forms in an amount of from 0.1% to 10% AUC as compared to the area of the compound of Formula XIV. In some embodiments, the compound of Formula XVII forms in an amount of from 0.1% to 5% AUC as compared to the area of the compound of Formula XIV. In some embodiments, the compound of Formula XVII forms in an amount of from 0.1% to 3% AUC as compared to the area of the compound of Formula XIV. In some embodiments, the compound of Formula XVII forms in an amount of 0.1%, or 0.15%, or 0.25%, or 0.5%, or 1%, or 2%, or 3%, or 4%, or 5%, or 6%, or 7%, or 8%, or 9%, or 10%, or any range of temperature values thereof, AUC as compared to the area of the compound of Formula XIV. In some embodiments, the compound of Formula XVII forms in a trace amount.

In some embodiments, a compound of Formula XV is reacted with a compound of Formula XVI to provide a compound of Formula XIV, wherein the compound of Formula XIV is isolated containing less than 3%, or less than 2%, or less than 1%, or less than 0.5%, less than 0.25%, or less than 0.15%, or any range of values thereof, AUC of the compound of Formula XVII. In some embodiments, the compound of Formula XIV is isolated containing less than 1% AUC of the compound of Formula XVII. In some embodiments, the compound of Formula XIV is isolated containing less than 0.5% AUC of the compound of Formula XVII. In some embodiments, the compound of Formula XIV is isolated containing less than 0.25% AUC of the compound of Formula XVII. In some embodiments, the compound of Formula XIV is isolated containing less than 0.15% AUC of the compound of Formula XVII. In some embodiments, the compound of Formula XIV is isolated containing undetectable amounts AUC of the compound of Formula XVII.

In another aspect, the present disclosure relates to a process for preparing a compound of Formula XIVb:

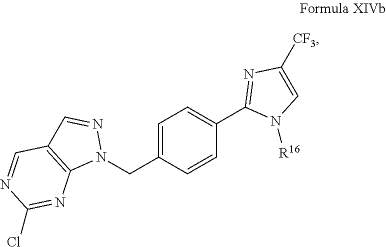

comprising reacting a compound of Formula Xb:

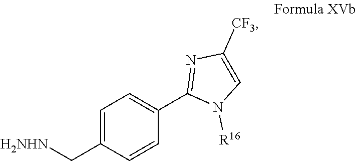

or a salt thereof, with a compound of Formula XVI:

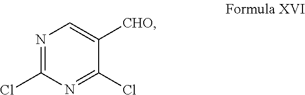

to form a compound of Formula XIVb.

In some embodiments, the compound of Formula Xb is reacted with the compound of Formula XVI in the presence of a solvent and a base to form a compound of Formula XIVb. In some embodiments, the solvent is selected from, but is not limited to, methanol, ethanol, isopropanol, and tetrahydrofuran. In some embodiments, the solvent is methanol. In some embodiments, the solvent is ethanol. In some embodiments, the solvent is isopropanol. In some embodiments, the solvent is tetrahydrofuran.

In some embodiments, the base is an amine base. In some embodiments, the amine base is triethylamine, tributylamine, 2,6-lutidine, N,N-diisopropylethylamine, or 1,8-diazabicyclo[5.4.0]undec-7-ene (DBU), and 1,5-diazabicyclo[4.3.0]non-5-ene (DBN). In some embodiments, the base is triethylamine. In some embodiments, the base is N,N-diisopropylethylamine. In some embodiments, the base is 2,6-lutidine.

In some embodiments, Formula XIVb is Formula XIV:

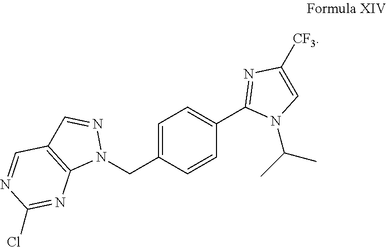 [SMILES: CC(C)n1cc(C(F)(F)F)nc1-c1ccc(Cn2ncc3cnc(Cl)nc32)cc1]

In some embodiments, Formula Xb is Formula X:

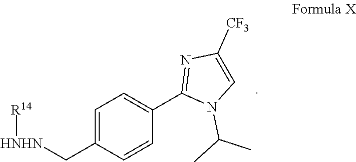

In another aspect, the present disclosure relates to a process comprising reacting the compound of Formula XIV with a compound of Formula XXI

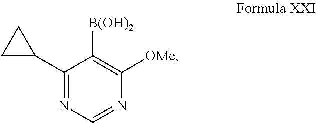 [SMILES: COc1ncnc(C2CC2)c1B(O)O]

to obtain a compound of Formula VI

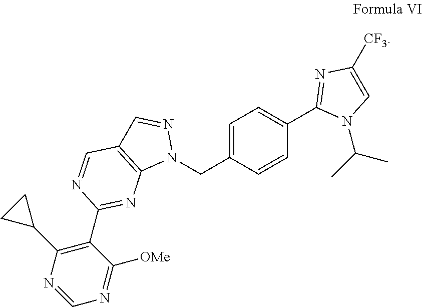 [SMILES: COc1ncnc(C2CC2)c1-c1ncc2cnn(Cc3ccc(-c4nc(C(F)(F)F)cn4C(C)C)cc3)c2n1]

In some embodiments, a compound of Formula XIV is reacted with a compound of Formula XXI in the presence of a palladium catalyst to form a compound of Formula VI. In some embodiments, the palladium catalyst is (dppf)PdCl₂. In some embodiments, the palladium catalyst is P(t-Bu)₃Pd. In some embodiments, the palladium catalyst is PdCl₂(dtbpf). In some embodiments, the palladium catalyst is Pd(OAc)₂ with A-PHOS. In some embodiments, the palladium catalyst is Pd-168.

In some embodiments, a compound of Formula XIV is reacted with a compound of Formula XXI in the presence of a solvent to form a compound of Formula VI. In some embodiments, the solvent is selected from, but not limited to, 1,4-dioxane, water, THF, 2-methyltetrahydrofuran, and mixtures thereof. In some embodiments, the solvent is 1,4-dioxane. In some embodiments, the solvent is water. In some embodiments, the solvent is THF. In some embodiments, the solvent is 2-methyltetrahydrofuran. In some embodiments, the solvent is a mixture of 1,4-dioxane and water.

In some embodiments, a compound of Formula XIV is reacted with a compound of Formula XXI in the presence of a base to form a compound of Formula VI. In some embodiments, the base is selected from sodium carbonate, potassium carbonate, and cesium carbonate. In some embodiments, the base is cesium carbonate.

In some embodiments, a compound of Formula XV is reacted with a compound of Formula XVI to form a compound of Formula XIV, and the compound of Formula XIV is reacted with a compound of Formula XXI to form a compound of Formula VI, wherein the compound of Formula VI comprises a trace amount of a compound of Formula XX. In some embodiments, a compound of Formula XIV is reacted with a compound of Formula XXI to form a compound of Formula VI, wherein the compound of Formula VI comprises a trace amount of a compound of Formula XX.

In another aspect, the present disclosure relates to a process comprising mixing the compound of Formula VI with gentisic acid in a solvent to form a compound of Formula XVIII.

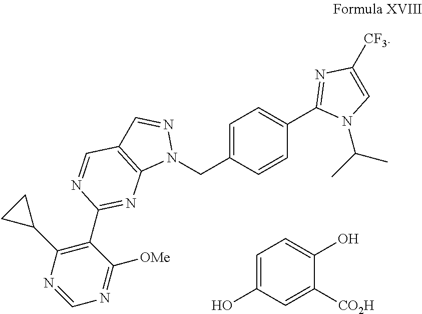 [SMILES: COc1ncnc(C2CC2)c1-c1ncc2cnn(Cc3ccc(-c4nc(C(F)(F)F)cn4C(C)C)cc3)c2n1 ; O=C(O)c1cc(O)ccc1O]

In some embodiments, the compound of Formula XVIII is a co-crystal.

In some embodiments, the compound of Formula VI is mixed with gentisic acid in the presence of a solvent to provide a compound of Formula XVIII. In some embodiments, the solvent is selected from, but is not limited to, ethyl acetate, heptane, acetone, isopropanol, and combinations thereof. In some embodiments, the solvent is isopropanol. In some embodiments, the solvent is heptane. In some embodiments, the solvent is a mixture of isopropanol and heptane.

In some embodiments, the compound of Formula XVIII is isolated from the solvent. In some embodiments, the compound of Formula XVIII is isolated from the solvent by precipitation. In some embodiments, the compound of Formula XVIII is isolated from the solvent by evaporation. In some embodiments, the compound of Formula XVIII is isolated from the solvent by crystalization. In some embodiments, the compound of Formula XVIII is re-crystallized using one or more solvents.

In some embodiments, the compound of Formula VI can be mixed with gentisic acid using the methods disclosed in U.S. Appl. Publication No. 2022/0162213, the disclosure of which is incorporated by reference.

In another aspect, the present disclosure relates to a process for preparing a compound of Formula XXX:

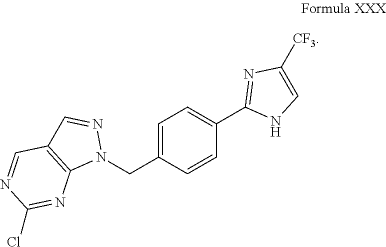 [SMILES: FC(F)(F)c1c[nH]c(-c2ccc(Cn3ncc4cnc(Cl)nc43)cc2)n1]

comprising:
reacting a compound of Formula XXVIV:

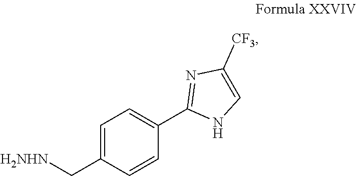

or a salt thereof,
with a compound of Formula XVI

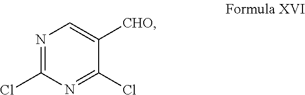

to form a compound of Formula XXX.

In some embodiments, the compound of Formula XXVIV is reacted with the compound of Formula XVI in the presence of a solvent and a base. In some embodiments, the solvent is selected from ethanol, isopropanol, or THF. In some embodiments, the base is an amine base. In some embodiments, the base is selected from triethylamine and diisopropylethylamine. In some embodiments, the reaction is conducted at a temperature of between −20° C. and 25° C.

In some embodiments, the process further comprises reacting the compound of Formula XXX with a compound of Formula XXI:

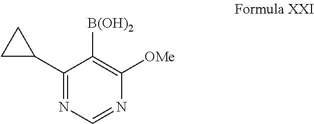 [SMILES: COc1ncnc(C2CC2)c1B(O)O]

to form a compound of Formula XXXI:

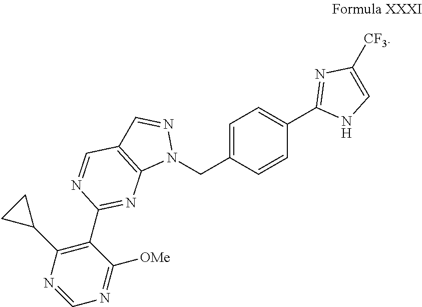 [SMILES: COc1ncnc(C2CC2)c1-c1ncc2cnn(Cc3ccc(-c4nc(C(F)(F)F)c[nH]4)cc3)c2n1]

In some embodiments, the process further comprises reacting the compound of Formula XXXI with

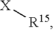

wherein X is a leaving group and R¹⁵ is an alkyl group;
to form a compound of Formula XXXII:

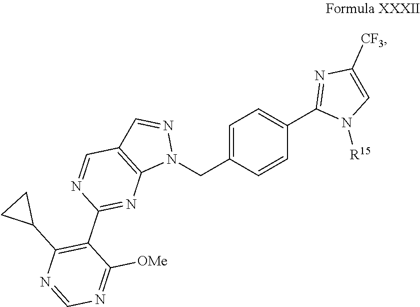

or a salt thereof.

In some embodiments, R¹⁵ is an isopropyl group. In some embodiments, R¹⁵ is a methyl group.

In another aspect, the present disclosure relates to a process for preparing a compound of Formula XXVIV:

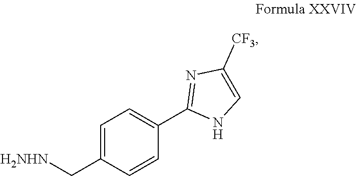

or a salt thereof,
comprising:
   reacting a compound of Formula XXVIII

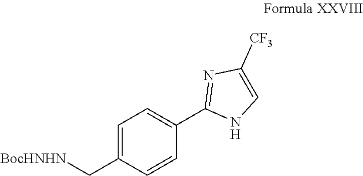

with acid to form a compound of Formula XXVIV or a salt thereof.

In some embodiments, the acid is selected from hydrochloric acid (HCl), hydrobromic acid (HBr), methanesulfonic acid, toluenesulfonic acid, trifluoroacetic acid, and trifluoromethanesulfonic acid. In some embodiments, the reaction is conducted at a temperature of between 0 and 75° C.

In another aspect, the present disclosure relates to a process for preparing a compound of Formula XXVIII:

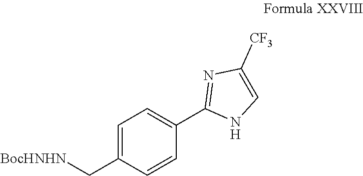

or a salt thereof,
comprising:
   reacting a compound of Formula XXVII:

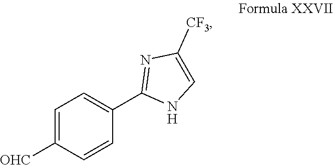 [SMILES: O=Cc1ccc(-c2nc(C(F)(F)F)c[nH]2)cc1]

with tert-butyl carbazate and a reducing agent to form a compound of Formula XXVIII.

In some embodiments, the compound of Formula XXVII is reacted with tert-butyl carbazate in the presence of hydrogen and a transition metal catalyst.

### Definitions

As used herein, the singular form “a,” “an,” and “the” includes plural references unless indicated otherwise. Use of the term “or” herein is not meant to imply that alternatives are mutually exclusive.

In this application, the use of “or” means “and/or” unless expressly stated or understood by one skilled in the art. In the context of a multiple dependent claim, the use of “or” refers back to more than one preceding independent or dependent claim.

The term “about,” as used herein, includes the recited number ±10%. Thus, “about 10” means 9 to 11. As is understood by one skilled in the art, reference to “about” a value or parameter herein includes (and describes) instances that are directed to that value or parameter per se. For example, description referring to “about X” includes description of “X.”

For the purpose of the present disclosure, the term “leaving group” or “LG” refers to an atom or group of atoms that becomes detached from an atom or group of atoms in what is considered to be the residual or main part of the molecule in a specified reaction. Non-limiting exemplary leaving groups include —Cl, —I, —Br, —OTf, —OMs, and —OTs.

For the purpose of the present disclosure, the term “halo” as used by itself or as part of another group refers to a halogen atom. Non-limiting exemplary halo groups include fluoro, chloro, bromo, and iodo.

For the purpose of the present disclosure, the term “cyano” as used by itself or as part of another group refers to —CN.

For the purpose of the present disclosure, the term “hydroxy” as used by itself or as part of another group refers to —OH.

For the purpose of the present disclosure, the term “amino” as used by itself or as part of another group refers to —NH₂.

For the purpose of the present disclosure, the term “alkyl” as used by itself or as part of another group refers to a straight- or branched-chain aliphatic hydrocarbon containing one to twelve carbon atoms (i.e., C₁₋₁₂ alkyl) or the number of carbon atoms designated (i.e., a C₁ alkyl such as methyl, a C₂ alkyl such as ethyl, a C₃ alkyl such as propyl or isopropyl, etc.). The alkyl group can be suitably chosen from a straight chain C₁₋₁₀ alkyl group, a branched chain C₃₋₁₀ alkyl group, a straight chain C₁₋₆ alkyl group, a branched chain C₃₋₆ alkyl group, a straight chain C₁₋₄ alkyl group, a branched chain C₃₋₄ alkyl group, a straight or branched chain C₃₋₄ alkyl group. The alkyl group can be partially or completely deuterated, i.e., one or more hydrogen atoms of the alkyl group are replaced with deuterium atoms. Non-limiting exemplary C₁₋₁₀ alkyl groups include methyl (including —CD₃), ethyl, propyl, isopropyl, butyl, sec-butyl, tert-butyl, iso-butyl, 3-pentyl, hexyl, heptyl, octyl, nonyl, and decyl. Non-limiting exemplary C₁₋₄ alkyl groups include methyl, ethyl, propyl, isopropyl, butyl, sec-butyl, tert-butyl, and iso-butyl.

For the purpose of the present disclosure, the term “alkoxy” as used by itself or as part of another group refers to an optionally substituted alkyl, optionally substituted cycloalkyl, optionally substituted heterocyclo, optionally substituted alkenyl or optionally substituted alkynyl attached to a terminal oxygen atom. The alkoxy group can be chosen from a C₁₋₄ alkoxy group and a C₁₋₄ alkyl attached to a terminal oxygen atom, e.g., methoxy, ethoxy, and tert-butoxy.

For the purpose of the present disclosure, the term “aryl” as used by itself or as part of another group refers to a monocyclic or bicyclic aromatic ring system having from six to fourteen carbon atoms (i.e., C₆₋₁₄ aryl). The bicyclic aromatic ring system can be fused. The aryl group can be chosen from a C₆₋₁₄ aryl group and a C₆₋₁₀ aryl group. Non-limiting exemplary aryl groups include phenyl (abbreviated as “Ph”), naphthyl, phenanthryl, anthracyl, indenyl, azulenyl, biphenyl, biphenylenyl, and fluorenyl groups. The aryl group can be chosen from phenyl or naphthyl. The aryl group can be phenyl.

For the purpose of the present disclosure, the term “optionally substituted aryl” as used herein by itself or as part of another group means that the aryl as defined above is either unsubstituted or substituted with one to five substituents independently selected from the group consisting of hydrogen, halo, hydroxy, alkoxy, alkyl, alkoxycarbonyl, cycloalkyl, optionally substituted aryl, and optionally substituted heteroaryl. The optional substituents can be alkoxy and cycloalkyl.

For the purpose of the present disclosure, the term “heteroaryl” or “heteroaromatic” refers to monocyclic and bicyclic aromatic ring systems having 5 to 14 ring atoms (i.e., C₅₋₁₄ heteroaryl) and 1, 2, 3, or 4 heteroatoms independently chosen from oxygen, nitrogen or sulfur. The heteroaryl group can be chosen from a C₅₋₁₄ heteroaryl group and a C₃₋₆ heteroaryl group. The heteroaryl can have three heteroatoms, two heteroatoms, or one heteroatom. The heteroaryl can be a C₅ heteroaryl, or a C₆ heteroaryl. Non-limiting exemplary heteroaryl groups include thienyl, benzo[b]thienyl, naphtho[2,3-b]thienyl, thianthrenyl, furyl, benzofuryl, pyranyl, isobenzofuranyl, benzooxazonyl, chromenyl, xanthenyl, 2H-pyrrolyl, pyrrolyl, imidazolyl, pyrazolyl, pyridyl, pyrazinyl, pyrimidinyl, pyridazinyl, isoindolyl, 3H-indolyl, indolyl, indazolyl, purinyl, isoquinolyl, quinolyl, phthalazinyl, naphthyridinyl, cinnolinyl, quinazolinyl, pteridinyl, 4aH-carbazolyl, carbazolyl, β-carbolinyl, phenanthridinyl, acridinyl, phenanthrolinyl, phenazinyl, thiazolyl, isothiazolyl, phenothiazolyl, isoxazolyl, furazanyl, triazolyl, tetrazolyl, and phenoxazinyl. The heteroaryl can be chosen from thienyl (e.g., thien-2-yl and thien-3-yl), furyl (e.g., 2-furyl and 3-furyl), pyrrolyl (e.g., 1H-pyrrol-2-yl and 1H-pyrrol-3-yl), imidazolyl (e.g., 2H-imidazol-2-yl and 2H-imidazol-4-yl), pyrazolyl (e.g., 1H-pyrazol-3-yl, 1H-pyrazol-4-yl, and 1H-pyrazol-5-yl), pyridyl (e.g., pyridin-2-yl, pyridin-3-yl, and pyridin-4-yl), pyrimidinyl (e.g., pyrimidin-2-yl, pyrimidin-4-yl, and pyrimidin-5-yl), thiazolyl (e.g., thiazol-2-yl, thiazol-4-yl, and thiazol-5-yl), isothiazolyl (e.g., isothiazol-3-yl, isothiazol-4-yl, and isothiazol-5-yl), oxazolyl (e.g., oxazol-2-yl, oxazol-4-yl, and oxazol-5-yl) isoxazolyl (e.g., isoxazol-3-yl, isoxazol-4-yl, and isoxazol-5-yl), triazolyl (e.g., 1,2,4-triazolyl and 1,2,3-triazolyl). The term “heteroaryl” is also meant to include possible N-oxides. Exemplary N-oxides include pyridyl N-oxide.

For the purpose of the present disclosure, the term “optionally substituted heteroaryl” as used by itself or as part of another group means that the heteroaryl as defined above is either unsubstituted or substituted with one to four substituents, e.g., one or two substituents, independently selected from the group consisting of hydrogen, halo, hydroxy, alkoxy, alkyl, alkoxycarbonyl, cycloalkyl, optionally substituted aryl, and optionally substituted heteroaryl. The optional substituents can be alkoxy and cycloalkyl.

For the purpose of the present disclosure, the term “alkoxycarbonyl” as used by itself or as part of another group refers to a carbonyl group, i.e., —C(═O)—, substituted by an alkoxy group. Non-limiting exemplary alkoxycarbonyl groups are —CO₂Me and —CO₂Et.

For the purpose of the present disclosure, the term “cycloalkyl” as used by itself or as part of another group refers to saturated and partially unsaturated (containing one or two double bonds) cyclic aliphatic hydrocarbons containing one to three rings having from three to twelve carbon atoms (i.e., C₃₋₁₂ cycloalkyl) or the number of carbons designated. The cycloalkyl group can have two rings, or one ring. The cycloalkyl group can be chosen from a C₃₋₈ cycloalkyl group and a C₃₋₆ cycloalkyl group. The cycloalkyl group can contain one or more carbon-to-carbon double bonds or one carbon-to-carbon double bond. Non-limiting exemplary cycloalkyl groups include cyclopropyl, cyclobutyl, cyclopentyl, cyclohexyl, cycloheptyl, cyclooctyl, norbornyl, decalin, adamantyl, cyclohexenyl, and spiro[3.3]heptane.

For the purpose of the present disclosure, the term “amine protecting group” as used herein refers to a group that blocks, i.e., protects, an amine functionality while reactions are carried out on other functional groups or parts of the molecule. Those skilled in the art will be familiar with the selection, attachment, and cleavage of amine protecting groups and will appreciate that different amine protective groups are known in the art, the suitability of one amine protective group or another being dependent on the particular synthetic scheme planned. Treatises on the subject are available for consultation, such as Wuts, “Greene's Protective Groups in Organic Synthesis”, 5th Ed., J. Wiley & Sons, Inc., NY, 2014. Suitable amine protecting groups include, but are not limited to, t-butyloxycarbonyl (Boc), fluorenylmethoxycarbonyl (Fmoc), benzyloxycarbonyl (Cbz), acetyl, trifluoroacetamide, phthalimide, benzyl, trityl, benzylideneamine, and toluenesulfonate.

For the purpose of the present disclosure, the term “base” as used herein refers to an organic proton acceptor. Non-limiting bases include “amine bases”, such as non-nucleophilic tertiary amines, e.g., triethylamine, N,N-diisopropylethylamine, N-methylmorpholine, and nitrogen-containing heteroaromatic groups such as pyridine, and derivatives of pyrindine, e.g., 2,4,6-collidine and 2,6-lutidine.

For the purpose of the present disclosure, the term “coupling” or “coupled” as used herein refers to a chemical reactions where two fragments or compounds are joined together through a carbon-carbon bond, optionally with the aid of a catalyst, such as a metal catalyst (e.g., a palladium catalyst). The coupling reactions disclosed herein can occur in the presence of a palladium catalyst.

For the purpose of the present disclosure, the term “sulfonating agent” as used herein refers to a reagent, or combination of reagents, used to form a bond between an oxygen atom and the sulfur atom in a sulfonate group. A sulfonating agent can be used to form a leaving group, such as a triflate group, a mesylate group, or a tosylate group. A triflate group refers to —SO₃CF₃, or —OTf A mesylate group refers to —SO₃CH₃, or —OMs. A tosylate group refers to —SO₃C₆H₄CH₃, or —OTs. Sulfonating agents are well known in the art. Any sulfonating agent known in art can be used to form a leaving group in the Compounds of the Disclosure and the processes disclosed herein. Non-limiting exemplary sulfonating agents include toluenesulfonic anhydride, toluenesulfonyl chloride, methansulfonic anhydride, methansulfonyl chloride, trifluoromethanesulfonic anhydride, and trifluoromethanesulfonyl chloride.

For the purpose of the present disclosure, the term “alkylating agent” as used herein refers to a reagent, or combination of reagents, used to replace a hydrogen with an alkyl group. An alkylating agent can be used to replace a hydrogen that is bonded to a nitrogen with an alkyl group. Non-limiting exemplary alkylating agents include 2-iodopropane, 2-bromopropane, 2-isopropyl mesylate, and 2-isopropyl tosylate.

For the purpose of the present disclosure, the term “reducing,” “reduced.” or “reduction” as used herein refers to a chemical process that results in the gain of electrons or a decrease in the oxidation state of an atom or atoms in a compound. An example of “reducing” occurs when a trflate group (—OTf) is removed from a compound of Formula V to form a compound of Formula VI, as disclosed herein. Another example of “reducing” occurs when a cyano group in a compound of Formula XII is converted to an aldehye group in the compound of Formula XI. Compounds can be reduced, for example, in the presence of a palladium catalyst.

For the purpose of the present disclosure, the term “reducing agent” as used herein refers to a reagent, or combination of reagents, used to donate an electron to an electron recipient. A reducing agent can be used, for example, to convert a cyano group in a compound of Formula XII to an aldehye group in the compound of Formula XI. A reducing agent can also be used, for example, to remove a triflate group from a compound of Formula V to form a compound of Formula VI. Non-limiting exemplary reducing agents include sodium cyanoborohydride, diisobutylaluminum hydride, palladium and a trialkylsilane, hydrogen and a palladium catalyst, lithium aluminum hydride, sodium borohydride, and sodium bis(2-methoxyethoxy)aluminium hydride.

For the purpose of the present disclosure, the term “palladium catalyst” as used herein refers to a reagent, or combination of reagents, containing palladium that is used to speed up chemical reactions, such as hydrogenation, dehydrogenation, and carbon-carbon bond formation, such as in coupling reactions. A palladium catalyst can be used to catalyze, or speed up, a reaction to remove a triflate group from a compound of Formula V to form a compound of Formula VI. A palladium catalyst can also be used to catalyze, or speed up, a coupling reaction between a compound of Formula XIV and a compound of Formula XXI, or a coupling reaction between a compound of Formula IX and a compound of Formula XXI. Non-limiting exemplary palladium catalysts include (dppf)PdCl₂, Pd₂(dba)₃, and Pd(PPh₃)₄.

For the purpose of the present disclosure, the term “% AUC” as used herein means percent area under the curve. The % AUC refers to a method for determining relative amounts of particular products following a chemical reaction as shown by the area under a curve resulting from high performance liquid chromatography (HPLC) purification and/or measurements. For example, a reaction that results in a compound of Formula XVII in an amount of less than 5% AUC as compared to a compound of Formula XIV, means that the compound of Formula XVII is formed in an amount that is less than 5% of the amount of a compound Formula XIV that is formed, as shown by the area under the curve following normalized integration of the peaks corresponding to Formula XVII and Formula XIV.

In another aspect, the present disclosure relates to a process for preparing a compound of Formula XVIII as shown in Scheme 1:

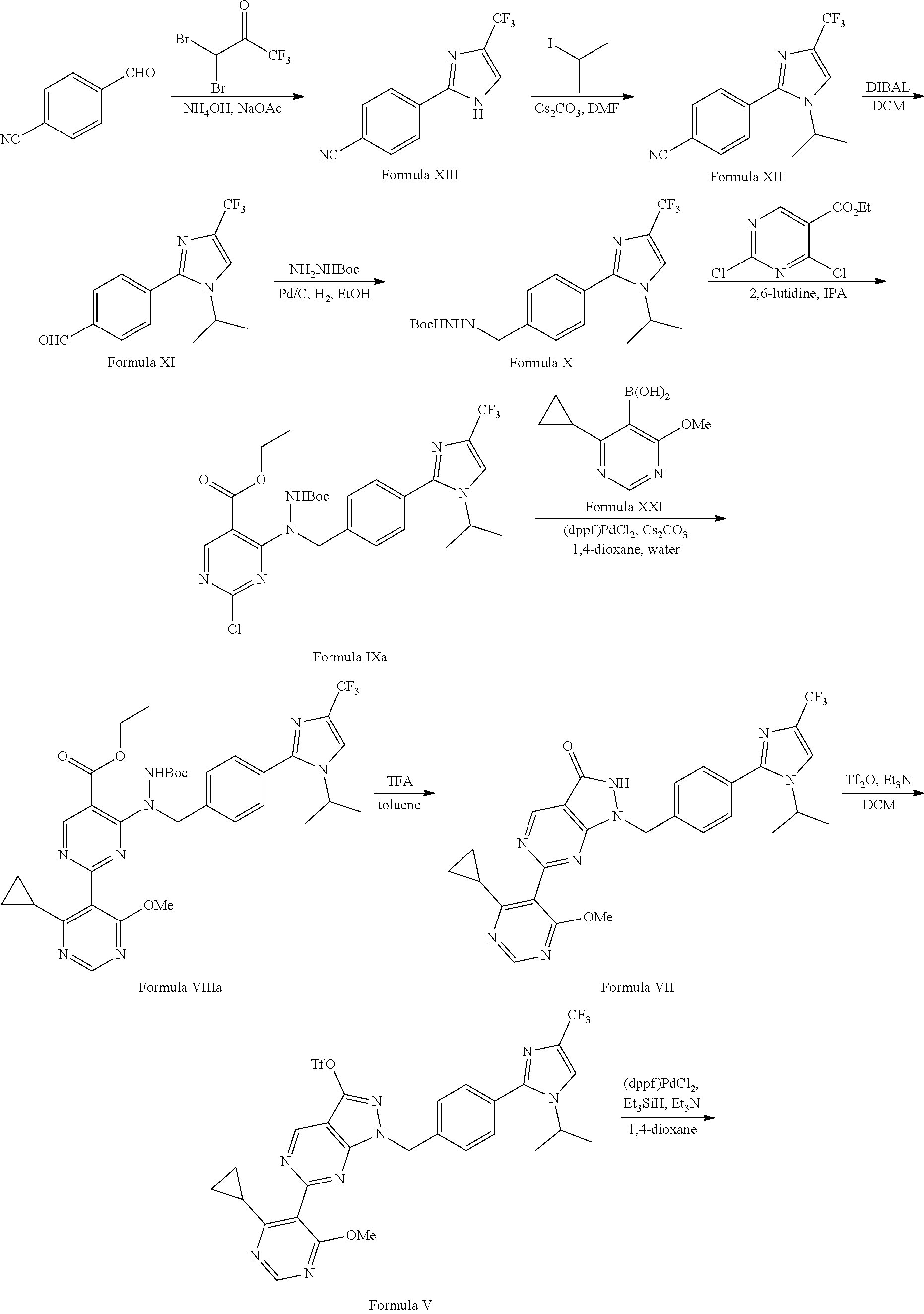

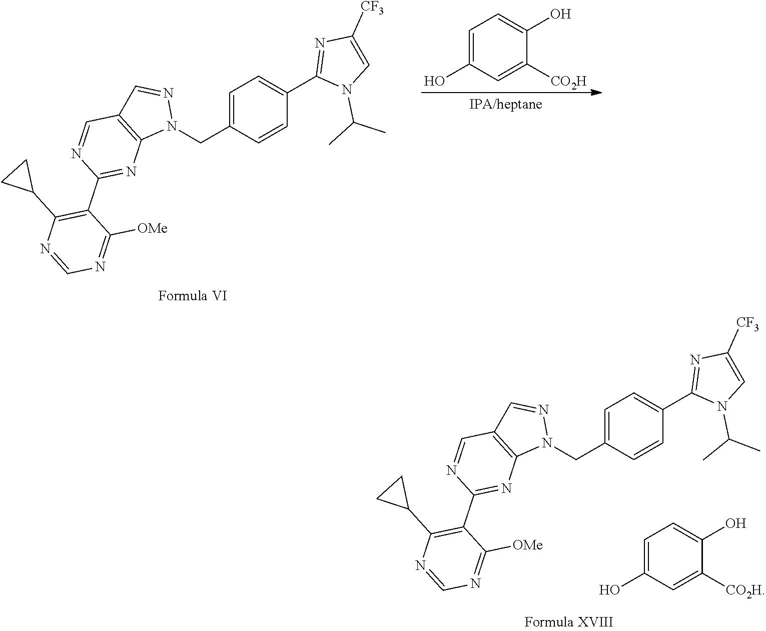 [SMILES: COc1ncnc(C2CC2)c1-c1ncc2cnn(Cc3ccc(-c4nc(C(F)(F)F)cn4C(C)C)cc3)c2n1 ; O=C(O)c1cc(O)ccc1O ; COc1ncnc(C2CC2)c1-c1ncc2cnn(Cc3ccc(-c4nc(C(F)(F)F)cn4C(C)C)cc3)c2n1 ; O=C(O)c1cc(O)ccc1O]

In another aspect, the present disclosure relates to a process for preparing a compound of Formula XVIII as shown in Scheme 2:

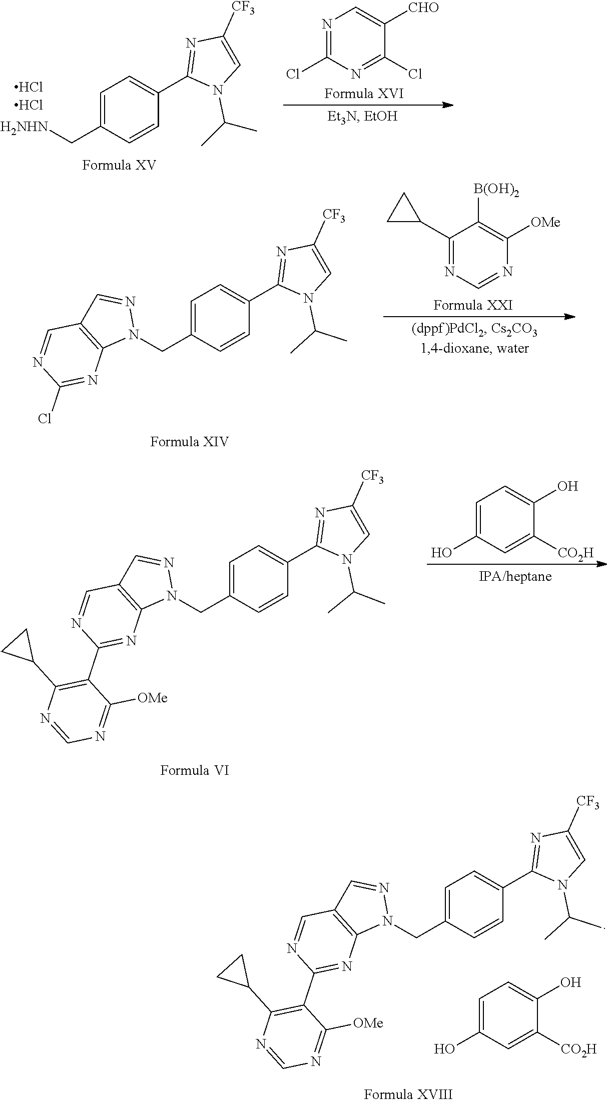

In another aspect, the present disclosure relates to a process for preparing a compound of Formula XI as shown in Scheme 3:

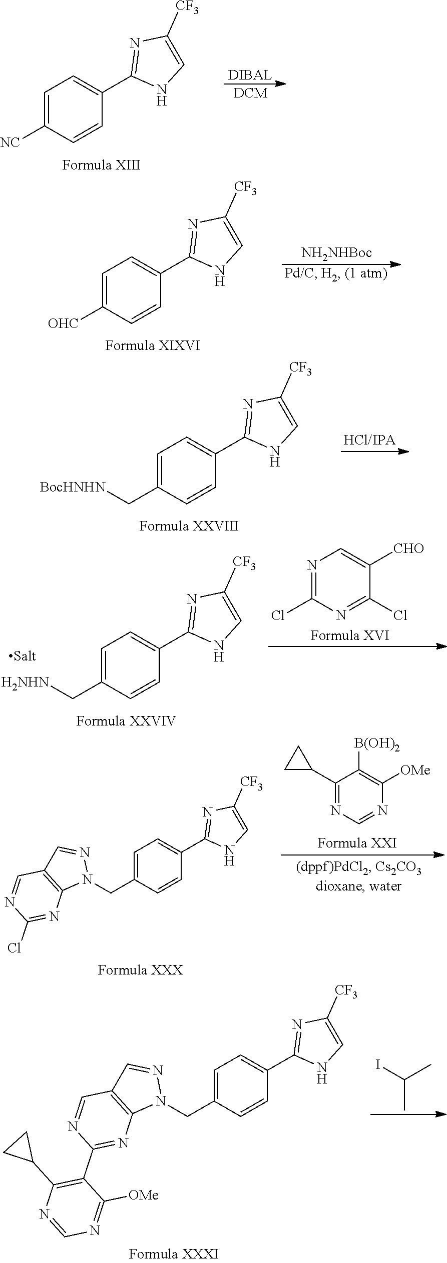

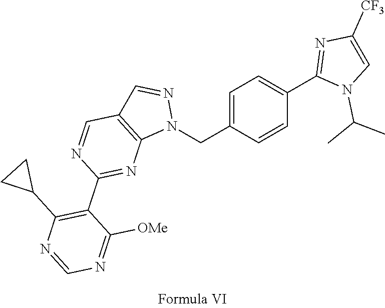 [SMILES: COc1ncnc(C2CC2)c1-c1ncc2cnn(Cc3ccc(-c4nc(C(F)(F)F)cn4C(C)C)cc3)c2n1]

### EXAMPLES

### Example 1: Synthesis of 4-(4-(trifluoromethyl)-1H-imidazol-2-yl)benzonitrile (XIII)

A 100 L reactor was degassed and refilled with N2 three times. Water (61.8 kg) and NaOAc (18.8 kg, 246.3 mol, 4.0 eq) were charged. Agitation was started and 1,1-dibromo-3,3,3-trifluoroacetone (30.9 kg, 258.6 mol, 2.0 eq) was added. The batch was heated to reflux, stirred for 40 min and then cooled to 20-25° C. The resulting light yellow solution was added to a solution of 4-formylbenzonitrile (7.5 kg, 57.2 mol) in MeOH (88.9 kg, 15.0 vol) and 25% aqueous NH₄OH (35.0 kg, 514.7 mol, 9.0 eq) in a 300 L reactor, keeping the inner temperature ≤45° C. The mixture was cooled to 20-25° C. and stirred for 16 h. Water (24.5 kg) was added carefully to the mixture and stirred for 1 h. The resulting slurry was filtered and rinsed with 50% MeOH/H₂O. The wet cake was dried under vacuum at 40° C. to afford 9.3 kg of 4-(4-(trifluoromethyl)-1H-imidazol-2-yl)benzonitrile (XIII) as a yellow solid. (68.6% yield, 98.9% purity by HPLC). LC-MS (ESI+): m/z 238.2 [M+H]+; ¹H NMR (400 MHz, DMSO-d₆) δ 13.49 (br, 1H), 8.19-8.11 (m, 2H), 8.06 (q, J=1.3 Hz, 1H), 8.01-7.93 (m, 2H).

### Example 2: Synthesis of 4-(1-isopropyl-4-(trifluoromethyl)-1H-imidazol-2-yl)benzonitrile (XII)

A 200 L reactor was degassed and refilled with N2 three times, then 4-(4-(trifluoromethyl)-1H-imidazol-2-yl)benzonitrile (XIII, 10.4 Kg, 43.8 mol) and acetonitrile (66.4 kg, 8.0 vol) were charged. Agitation was started and to the resulting solution was added Cs₂CO₃ (42.9 kg, 131.5 mol, 3.0 eq) and 2-iodopropane (37.3 kg, 219.2 mol, 5.0 eq). The batch was heated to 74-77° C. and stirred for 4 h. The batch was then cooled down to 20° C. and filtered. The filtrate was concentrated under vacuum at 40-45° C. to a minimum stirrable volume. The batch was then diluted with ethyl acetate (75.6 kg, 8.0 vol) and washed successively with water (21.0 kg) and brine (14.3 kg). The solvent was removed under reduced pressure at 40-45° C., then the resulting solids were triturated with n-heptane (35.7 kg, 5.0 vol) for 1 h and filtered. The filter cake was dried in a vacuum oven at 45° C. to afford 10.6 kg of 4-(1-isopropyl-4-(trifluoromethyl)-1H-imidazol-2-yl)benzonitrile (XII) as a yellow solid (86.6% yield, 96.9% purity by HPLC). LC-MS (ESI+): m/z 280.3 [M+H]+; ¹H NMR (400 MHz, CDCl₃) δ 7.83-7.75 (m, 2H), 7.75-7.66 (m, 2H), 7.48 (q, J=1.2 Hz, 2H), 4.55 (hept, J=6.7 Hz, 1H), 1.50 (d, J=6.7 Hz, 6H).

### Example 3: Synthesis of 4-(1-isopropyl-4-(trifluoromethyl)-1H-imidazol-2-yl)benzaldehyde (XI)

A 200 L reactor was degassed and refilled with N2 three times. To the reactor was charged 4-(1-isopropyl-4-(trifluoromethyl)-1H-imidazol-2-yl)benzonitrile (XII, 10.4 Kg, 37.2 mol) followed by dichloromethane (137.8 kg, 10.0 vol). Agitation was started and the resulting solution was cooled to −15° C. To the batch was added 1.0 M DIBAL-H solution in hexanes (38.4 kg, 55.9 mol, 1.5 eq) over 2 hours, maintaining the internal temperature ≤0° C. After the addition was complete, the batch was stirred at −10° C. for 1 h. The reaction was then slowly quenched into cold (0-5° C.) 5% aq. H₂SO₄ (10.4 kg, 106.0 mol, 2.85 eq, water: 197.6 kg) in a 500 L reactor, maintaining the inner temperature ≤15° C. The batch was then heated to 30° C. for 24 h and allowed to settle. The organic layer was separated and kept, and the aqueous layer was further extracted with dichloromethane (137.8 kg, 10.0 vol). The organic layers were combined, dried over Na₂SO₄ (15.0 kg) and filtered. The filtrate was concentrated under vacuum at 35-40° C. to a minimum volume then diluted with EtOH (71.0 kg). The resulting solution (81.5 kg solution containing 10.5 kg 4-(1-isopropyl-4-(trifluoromethyl)-1H-imidazol-2-yl)benzaldehyde (XI, 99.9% yield, 93.3% purity by HPLC) was taken forward directly into the following step. LC-MS (ESI+): m/z 283.3 [M+H]+; ¹H NMR (400 MHz, CDCl₃) δ 10.02 (s, 1H), 7.98-7.89 (m, 2H), 7.72-7.65 (m, 2H), 7.41 (q, J=1.2 Hz, 1H), 4.53 (hept, J=6.7 Hz, 1H), 1.43 (d, J=6.7 Hz, 6H).

### Example 4: Synthesis of tert-butyl 2-(4-(1-isopropyl-4-(trifluoromethyl)-1H-imidazol-2-yl)benzyl)hydrazine-1-carboxylate (X)

A 300 L reactor was degassed and refilled with N2 three times, then 4-(1-isopropyl-4-(trifluoromethyl)-1H-imidazol-2-yl)benzaldehyde (XI, 17.3 kg, 61.3 mol) was charged as a solution in EtOH (72.1 kg). Tert-butyl carbazate (8.5 kg, 64.3 mol, 1.05 eq) and AcOH (0.37 kg, 6.16 mol, 0.1 eq) were added to the batch as a solution in ethanol (81.8 kg). The batch was stirred at 25° C. for 2 hours to allow for formation of the intermediate hydrazone, then triethylamine (0.62 kg, 6.13 mol, 0.1 eq) was added. The batch was stirred for 30 min, then 10% Pd/C (2.3 kg) was charged under N2. The reactor was purged with hydrogen gas 3 times and the inner pressure was kept between 0.002-0.008 MPa. The reaction was stirred at 25° C. for 17 h. The reactor was purged with N2 3 times, and the reaction mixture was filtered through Celite and washed with EtOH (27.3 kg, 2.0 vol). The filtrate was concentrated under vacuum at 40-45° C. to a minimum volume, then ethyl acetate (138.1 L, 8.0 vol) was charged and the batch was washed with water (17.3 kg, 1.0 vol). The layers were separated, and the organic layer was concentrated under vacuum at 40-45° C. to a minimum volume. Ethyl acetate (31.1 kg, 2.0 vol) was then charged and the batch was heated to 50-55° C. To the resulting solution was carefully added n-heptane (138.4 L, 8.0 vol), maintaining the inner temperature not less than 50° C. The mixture was cooled to 20-25° C. over 2 hours, and then further cooled down to 0-5° C. The resulting slurry was stirred at 0-5° C. for 1 h, filtered, and washed with 20% v/v ethyl acetate/n-heptane (17.6 kg). The wet cake was dried under vacuum at 40-45° C. to afford 19.4 kg of tert-butyl 2-(4-(1-isopropyl-4-(trifluoromethyl)-1H-imidazol-2-yl)benzyl)hydrazine-1-carboxylate (X) as an off-white solid. (79.4% yield, 99.2% purity by HPLC). LC-MS (ESI+): m/z 399.3 [M+H]+; ¹H NMR (400 MHz, CDCl₃) δ 7.55-7.44 (m, 4H), 7.42 (q, J=1.2 Hz, 1H), 6.02 (s, 1H), 4.57 (hept, J=6.7 Hz, 1H), 4.27 (s, 1H), 4.06 (s, 2H), 1.49-1.43 (m, 15H).

### Example 5: Synthesis of ethyl 4-(2-(tert-butoxycarbonyl)-1-(4-(1-isopropyl-4-(trifluoromethyl)-1H-imidazol-2-yl)benzyl)hydrazineyl)-2-chloropyrimidine-5-carboxylate (IXa)

To a slurry of tert-butyl 2-(4-(1-isopropyl-4-(trifluoromethyl)-1H-imidazol-2-yl)benzyl)hydrazine-1-carboxylate (X, 27 kg, 1 equiv) in isopropanol (129.6 L, 4.8 vol) at 30±5° C. was added 2,6-lutidine (1.1 equiv). The addition line was flushed with isopropanol (5.4 L, 0.2 vol). A solution of ethyl 2,4-dichloropyrimidine-5-carboxylate (16.5 kg, 1.1 equiv) in isopropanol (135 L, 5 vol) was then added, keeping the temperature at 30±5° C. The clear solution was stirred for 3 h, then sampled for reaction completion by HPLC.

Once the reaction was completed, the batch was heated to 50±5° C., then water (270 L, 10 vol) was added over at least 1 h, keeping the batch temperature at 50±5° C. After complete addition of water, the batch was stirred at this temperature for at least 20 min, during which time solids precipitated. The batch was then cooled to 30±5° C., stirred for an additional 4 h, then filtered. The wet cake was washed with 50% isopropanol/water (81 L, 3 vol), then dried under vacuum at 55-65° C. for 8-10 hours to provide ethyl 4-(2-(tert-butoxycarbonyl)-1-(4-(1-isopropyl-4-(trifluoromethyl)-1H-imidazol-2-yl)benzyl)hydrazineyl)-2-chloropyrimidine-5-carboxylate (IXa) (34.3 kg, 86% yield, 99.4% purity by HPLC) as an off white solid. LC-MS (ESI+): m/z 583.2 [M+H]⁺; ¹H NMR (400 MHz, DMSO-d₆) δ 9.78 (s, 1H), 9.32 (br s, 0.24H), 8.33 (s, 1H), 8.17 (s, 1H), 7.57-7.36 (m, 4H), 5.20-4.96 (m, 1H), 4.87-4.64 (m, 1H), 4.54-4.31 (m, 1H), 4.24-4.22 (m, 2H), 1.51-1.18 (m, 18H).

### Example 6: Synthesis of ethyl 4-(2-(tert-butoxycarbonyl)-1-(4-(1-isopropyl-4-(trifluoromethyl)-1H-imidazol-2-yl)benzyl)hydrazineyl)-4′-cyclopropyl-6′-methoxy-[2,5′-bipyrimidine]-5-carboxylate (VIIIa)

To a mixture of ethyl 4-(2-(tert-butoxycarbonyl)-1-(4-(1-isopropyl-4-(trifluoromethyl)-1H-imidazol-2-yl)benzyl)hydrazineyl)-2-chloropyrimidine-5-carboxylate (IXa) (34 kg, 1 equiv) and cesium carbonate (56.8 kg, 3 equiv) in 1,4-dioxane (544 L, 16 vol) and water (136 L, 4 vol) at 30±5° C. was added (4-cyclopropyl-6-methoxypyrimidin-5-yl)boronic acid XXI (22.6 kg, 2 equiv). The batch was stirred for 15 minutes to generate a clear solution, then the batch was purged with nitrogen/argon for 30 minutes. Under the inert atmosphere, (dppf)PdCl₂ (3.2 kg, 7.5 mol %) was charged as a solid, and the batch was purged with nitrogen/argon for an additional 15 minutes. The batch was then heated to 60-65° C. for 2 h, then sampled for reaction completion periodically.

Once the reaction is complete, the batch was cooled to 30±5° C. and filtered over a bed of hyflow prepared with ethyl acetate. The filter cake was washed with ethyl acetate (68 L, 2 vol), then the combined filtrates were distilled to 3.5±0.5 volumes, keeping the temperature below 60° C. The batch was cooled to 30±5° C., then ethyl acetate (340 L, 10 vol) and water (340 L, 10 vol) were added and the batch was stirred for at least 30 min. The layers were settled and separated, then the organic layer was washed twice with a 5% brine solution (340 L, 10 vol×2).

Charcoal (Norit CGP super, 6.8 kg, 0.2 wt equiv) was charged to the organic layer as a slurry in ethyl acetate (102 L, 3 vol) between 30±5° C., and the batch was stirred for NLT 2 h. The batch was then filtered through a bed of hyflow prepared with ethyl acetate, and the filter cake was washed with ethyl acetate (102 L, 3 vol). The combined filtrates were distilled to 4.0±0.5 volumes, keeping the temperature below 60° C. Once the distillation was complete, n-heptane (340 L, 10 vol) was added to the batch, and the batch was heated to 60±5° C. to generate a clear solution. The batch is then cooled to 5+5° C. over at least 4 h, over which time the batch crystallized. The batch was stirred at this temperature for at least 12 h, then filtered, washed with n-heptane (102 L, 3 vol). The solids were dried under vacuum at 45-55° C. for 8 hours, to provide ethyl 4-(2-(tert-butoxycarbonyl)-1-(4-(1-isopropyl-4-(trifluoromethyl)-1H-imidazol-2-yl)benzyl)hydrazineyl)-4′-cyclopropyl-6′-methoxy-[2,5′-bipyrimidine]-5-carboxylate (VIIIa) (34.3 kg, 84%, 97.5% purity by HPLC) as a light brown solid. Note that this compound presents as rotamers by ¹H NMR. LC-MS (ESI+): m/z 697.2 [M+H]⁺; ¹H NMR (400 MHz, DMSO-d₆) δ 9.67 (s, 0.61H), 9.23 (s, 0.18H), 8.72-8.64 (m, 1H), 8.60-8.43 (m, 1H), 8.17 (s, 1H), 7.61-7.19 (m, 4H), 5.32-5.20 (m, 0.50H), 5.06-5.02 (m, 0.22H), 4.90-4.72 (m, 0.22H), 4.72-4.55 (m, 0.50H), 4.48-4.36 (m, 1H), 4.34-4.19 (m, 2H), 3.86 (s, 3H), 1.76-1.63 (m, 1H), 1.44-1.37 (m, 12H), 1.34-1.31 (m, 3H), 1.08-0.98 (m, 4.76H), 0.94-0.81 (m, 2H).

### Example 7: Synthesis of 6-(4-cyclopropyl-6-methoxypyrimidin-5-yl)-1-(4-(1-isopropyl-4-(trifluoromethyl)-1H-imidazol-2-yl)benzyl)-1,2-dihydro-3H-pyrazolo[3,4-d]pyrimidin-3-one (VII)

To a mixture of ethyl 4-(2-(tert-butoxycarbonyl)-1-(4-(1-isopropyl-4-(trifluoromethyl)-1H-imidazol-2-yl)benzyl)hydrazineyl)-4′-cyclopropyl-6′-methoxy-[2,5′-bipyrimidine]-5-carboxylate (VIIIa) (34.3 kg, 1 equiv) and toluene (343 L, 10 vol) at 30 10° C. was added TFA (56.2 kg, 10 equiv), keeping the temperature below 40° C. The batch was then heated to 50-55° C. and stirred at that temperature for at least 1 h. The batch is then sampled periodically for reaction completion.

Once the reaction is complete, the batch is cooled to 30-35° C. and diluted with ethyl acetate (412 L, 12 vol). Aqueous sodium carbonate solution (31.3 kg Na₂CO₃ in 6 vol water) is then cautiously added over time, keeping the batch temperature between 30-35° C. Upon completion of the addition, the batch is stirred for an additional 30 min, then the layers are separated. The aqueous layer is back extracted with ethyl acetate (172 L, 5 vol), and the combined organic layers are then washed with a 10% aqueous sodium chloride solution (172 L, 5 vol). The batch is then distilled to 3.0±0.5 vol under vacuum, keeping the internal temperature below 60° C. The batch was then cooled to 30±5° C. and n-heptane (343 L, 10 vol) is added to generate a slurry. The batch was then stirred at this temperature for at least 4 h, filtered, and washed with n-heptane (69 L, 2 vol). The wet cake was dried under vacuum at 50-60° C. for 8 hours to provide 6-(4-cyclopropyl-6-methoxypyrimidin-5-yl)-1-(4-(1-isopropyl-4-(trifluoromethyl)-1H-imidazol-2-yl)benzyl)-1,2-dihydro-3H-pyrazolo[3,4-d]pyrimidin-3-one (VII) (23.2 kg, 86%, 96.8% purity by HPLC) as a light brown solid. LC-MS (ESI+): m/z 551.2 [M+H]⁺; ¹H NMR (400 MHz, DMSO-d₆) δ12.31-11.43 (m, 1H), 9.28 (s, 1H), 8.69 (s, 1H), 8.16 (s, 1H), 7.52 (d, J=8.2 Hz, 2H), 7.39 (d, J=8.2 Hz, 2H), 5.52 (s, 2H), 4.50-4.30 (m, 1H), 3.86 (s, 3H), 1.77-1.62 (m, 1H), 1.38 (d, J=6.7 Hz, 6H), 1.08-0.99 (m, 2H), 0.88-0.86 (m, 2H).

### Example 8: Synthesis of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidin-3-yl trifluoromethanesulfonate (V)

To a solution of 6-(4-cyclopropyl-6-methoxypyrimidin-5-yl)-1-(4-(1-isopropyl-4-(trifluoromethyl)-1H-imidazol-2-yl)benzyl)-1,2-dihydro-3H-pyrazolo[3,4-d]pyrimidin-3-one (VII) (7.46 kg, 1 equiv) in dichloromethane (75 L, 10 vol) at 30±5° C. was added triethylamine (2.8 kg, 1.5 equiv), and the clear solution was cooled to 0±5° C.

Trifluoromethanesulfonic anhydride (5.74 kg, 1.5 equiv) was then carefully added to the batch, keeping the temperature between 0±5° C. The clear reaction mixture was stirred for at least 1 h at that temperature, then sampled periodically for reaction completion. Once the reaction is complete, water (75 L, 10 vol) is then cautiously added over time, keeping the batch temperature between 0±5° C. The batch is then warmed to 30±5° C., stirred for at least 15 min at this temperature, and the layers were separated. The aqueous layer was extracted with dichloromethane (37.3 L, 5 vol), then the combined organic layers were washed with water (75 L, 10 vol). The solvent was then distilled under vacuum to 3.0±0.5 volumes, keeping the temperature below 40° C. The batch was then carefully added into precooled (0-5° C.) n-heptane (149 L, 20 vol) over at least 3 hours. The resulting slurry is stirred at this temperature for at least 2 h, then warmed to 30±5° C. and stirred for an additional 3 h. The slurry is then filtered, and dried under vacuum at 40-45° C. for 8 hours to provide 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidin-3-yl trifluoromethanesulfonate (V) (8.5 kg, 92%, 93% purity by HPLC) as a light brown solid. LC-MS (ESI+): m/z 683.2 [M+H]⁺; 6 9.69 (s, 1H), 8.73 (s, 1H), 8.16 (s, 1H), 7.59-7.52 (m, 2H), 7.45 (d, J=8.3 Hz, 2H), 5.81 (s, 2H), 4.45-4.40 (m, 1H), 3.87 (s, 3H), 1.76-1.74 (m, 1H), 1.38 (d, J=6.6 Hz, 6H), 1.17-1.05 (m, 2H), 1.00-0.80 (m, 2H).

### Example 9: Synthesis of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (VI) from 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidin-3-yl trifluoromethanesulfonate (V)

To a stirred solution of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidin-3-yl trifluoromethanesulfonate (V) (7.2 kg, 1 equiv) in 1,4-dioxane (72 L, 10 vol) at 30±5° C. was added (dppf)PdCl₂ (230 g, 3 mol %). The batch was heated to 75-85° C., then a solution of triethylsilane (3.74 kg, 2.5 equiv) and triethylamine (1.3 kg, 1.2 equiv) in dioxane (14 L, 2 vol) was added into the reaction mixture, keeping the temperature between 75-85° C. The batch was heated at that temperature for 2 h, then sampled periodically for reaction completion.

Once the reaction is complete, the batch was distilled under vacuum to 2-3 volumes, then MTBE (144 L, 20 vol) and water (72 L, 10 vol) were added. The temperature of the batch was adjusted to 30±5° C., stirred for 30 min at that temperature, then filtered through a bed of hyflow. The resulting layers were then separated, and the organic layer was washed with water (72 L, 10 vol). The organic layer was treated with activated carbon (1.4 kg, 20% w/w) and the resulting slurry was held at 30±5° C. for at least 4 hours. The batch was filtered through a bed of hyflow, then distilled under vacuum to 5 volumes. The temperature of the batch was adjusted to 45-55° C., then n-heptane (108 L, 15 vol) was added, keeping the temperature in that range during the addition. After the addition was complete, the batch was stirred at that temperature for 30 min, then cooled to 30±5° C. The batch was stirred for an additional 2-3 hours at that temperature, then filtered, and the wet cake washed with n-heptane (14 L, 2 vol). The wet cake was dried under vacuum at 45-50° C. to provide a crude product (4.25 kg).

The crude product is then recrystallized using the following process. Volumes and equivalents used are in relation to the crude product, not the initial input of Formula V.

Crude Formula (VI) (4.25 kg, 1 equiv) was dissolved in methanol (34 L, 8 vol) and dichloromethane (8.5 L, 2 vol), then activated carbon (0.72 kg, 10% w/w) was added. The batch was stirred at 30±5° C. for NLT 2 h, then filtered through a bed of hyflow. The batch was distilled under vacuum to 2.5 volumes, then methanol (10.6 L, 2.5 vol) and triethylamine (4.2 L, 1 vol) were added and the temperature was adjusted to 50±5° C. To the clear solution was added water (21.2 L, 5 vol) over at least 1 h. The batch was stirred at that temperature for 30 min, then cooled to 0-5 C over at least 4 h. The resulting solids were filtered, washed with 50% methanol: water (8.5 L, 2 vol), then dried at 45-55° C. to provide 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (VI) (3.5 kg, 62%, 98.8% purity by HPLC) as a pale yellow solid. LC-MS (ESI+): m/z 535.2 [M+H]⁺; ¹H NMR (400 MHz, DMSO-d₆) δ 9.49 (s, 1H), 8.69 (s, 1H), 8.50 (s, 1H), 8.14 (s, 1H), 7.51 (d, J=7.8 Hz, 2H), 7.40 (d, J=7.8 Hz, 2H), 5.77 (s, 2H), 4.45-4.34 (m, 1H), 3.83 (s, 3H), 1.60-1. 67 (m, 1H), 1.35 (d, J=6.8 Hz, 6H), 1.04 (m, 2H), 0.83 (m, 2H).

### Example 10: Synthesis of 6-(4-cyclopropyl-6-methoxypyrimidin-5-yl)-1-(4-(1-isopropyl-4-(trifluoromethyl)-1H-Imidazol-2-yl)benzyl)-1H-pyrazolo[3,4-d]pyrimidine gentisic acid co-crystal (XVIII)

To a slurry of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (VI) (5.0 kg, 1 equiv) in isopropanol (30 L, 6 vol) at 30±5° C. was added gentisic acid (1.44 kg, 1 equiv). The resulting slurry was heated to 72.5+2.5° C. for 30 min to generate a clear solution. To the solution was charged n-heptane (50 L, 10 vol) over at least 2 h. After complete addition of n-heptane, the batch was stirred at 72.5+2.5° C. for 30 min, then cooled to 30±5° C. over 2-4 hours. The batch was stirred at that temperature for 16-20 hours, then filtered, washed with n-heptane (10 L, 2 vol) and dried under vacuum at 60 5° C. for 8 h to provide 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine gentisic acid co-crystal (XVIII) (5.82 kg, 90%, 99.1% purity by HPLC) as a white solid. LC-MS (ESI+): m/z 535.2 [M+H]+; ¹H NMR (400 MHz, DMSO-d₆) δ 13.736 (s, 1H), 10.642 (s, 1H), 9.509 (s, 1H), 9.131 (s, 1H), 8.709 (s, 1H), 8.520 (s, 1H), 8.156 (s, 1H), 7.528 (d, J=8 Hz, 2H), 7.425 (d, J=8 Hz, 2H), 7.153 (d, J=3.2 Hz, 1H), 6.956 (dd, J=8.8, 2.8 Hz, 1H), 6.780 (d, J=8 Hz, 1H), 5.792 (s, 2H), 4.409 (sept, J=6.8 Hz, 1H), 3.853 (s, 3H), 1.660 (m, 1H), 1.373 (d, J=6.8 Hz, 6H), 1.060 (m, 2H), 0.850 (m, 2H).

### Example 11: Synthesis of 2-(4-(hydrazineylmethyl)phenyl)-1-isopropyl-4-(trifluoromethyl)-1H-imidazole dihydrochloride (XV)

A 20 L four-neck flask was purged with N2 three times. tert-Butyl 2-(4-(1-isopropyl-4-(trifluoromethyl)-1H-imidazol-2-yl)benzyl)hydrazine-1-carboxylate (X) (1.25 Kg, 3.14 mol) and isopropanol (6.25 L, 5.0 vol) was charged. Agitation was started and the batch was heated to 75-80° C. 4M HCl in isopropanol (3.14 L, 12.56 mol, 4.0 eq) was added carefully over 2 hours. The batch was held with stirring at 75-80° C. for another 3 hours. Once the reaction was complete, the batch was cooled to 25-30° C. over the course of 4 hours. The resulting slurry was filtered and the solid was rinsed with isopropanol (2.5 L, 2 vol). The solid was collected and dried under vacuum at 40-45° C. for 22 hours to give 2-(4-(hydrazineylmethyl)phenyl)-1-isopropyl-4-(trifluoromethyl)-1H-imidazole dihydrochloride (XV) (1.10 kg, 94% yield, 98.6% AUC by HPLC) as an off-white solid. LC-MS (ESI+): m/z 298.8 (C₁₄H₁₇F₃N₄+H)⁺; ¹H NMR (400 MHz, DMSO-d₆) δ 8.21 (q, J=1.2 Hz, 1H), 7.59 (s, 4H), 6.89 (br, 5H), 4.48 (hept, J=6.5 Hz, 1H), 4.16 (s, 2H), 1.42 (d, J=6.6 Hz, 6H).

### Example 12: Synthesis of 6-chloro-1-(4-(1-isopropyl-4-(trifluoromethyl)-1H-imidazol-2-yl)benzyl)-1H-pyrazolo[3,4-d]pyrimidine (XIV)

A suitable reactor was charged with 2-(4-(hydrazineylmethyl)phenyl)-1-isopropyl-4-(trifluoromethyl)-1H-imidazole dihydrochloride (XV) (72 g, 1 equiv) and anhydrous ethanol (20 vol). The reaction mixture was cooled to −10° C. and inerted with argon gas. Triethylamine (81.2 mL, 3 equiv) was added to the reactor while keeping the temperature below −5° C. After complete addition, cool the mixture to −10° C. To the cooled batch was added 2,4-dichloropyrimidine-5-carboxaldehyde XVI (41.2 g, 1.2 equiv) as a solid in portions over 30-40 minutes. The batch was stirred at −10° C. until reaction completion as monitored by HPLC.

After completion of the reaction, it was allowed to warm to room temperature and the solvent was removed under vacuum on a rotovap (60° C.). All volume references that follow are with respect to the crude solids obtained after concentration.

The crude compound XIV was dissolved in DCM (5 vol) and washed with water (5 vol×2), followed by 2% aqueous HCl (5 vol). The organic layer was dried with sodium sulfate, then the solvent was removed under vacuum at 35° C. The crude compound was dissolved in ethanol (7 vol) and refluxed for 2 hours, then the batch was cooled to 75° C. and water (3 vol) was added over the course of an hour at the same temperature. The batch was then cooled to 20-25° C. over 4 hours, and then further cooled down to −10° C. The resulting slurry was stirred at −10° C. for 12 hrs. The solid was filtered and washed with a cold 1:1 ethanol/water mixture (1 vol). The solids were dried under vacuum at 50-55° C. to obtain 6-chloro-1-(4-(1-isopropyl-4-(trifluoromethyl)-1H-imidazol-2-yl)benzyl)-1H-pyrazolo[3,4-d]pyrimidine (XIV) as a yellow solid. (71.1 g, 87.1% yield, 94.7% AUC by HPLC containing <0.1% AUC of the N2 isomer Formula XVII). LC-MS (ESI+): m/z 420.9 [M+H]+; ¹H NMR (400 MHz, CDCl₃) δ 9.06 (s, 1H), 8.19 (s, 1H), 7.54-7.50 (m, 2H), 7.47 (d, J=8.4 Hz, 2H), 7.23 (s, 1H), 5.69 (s, 2H), 4.51 (hept, J=6.7 Hz, 1H), 1.43 (d, J=6.7 Hz, 6H).

### Example 13: Synthesis of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (VI) from 6-chloro-1-(4-(1-isopropyl-4-(trifluoromethyl)-1H-imidazol-2-yl)benzyl)-1H-pyrazolo[3,4-d]pyrimidine (XIV)

To a suitable reactor, 6-chloro-1-(4-(1-isopropyl-4-(trifluoromethyl)-1H-imidazol-2-yl)benzyl)-1H-pyrazolo[3,4-d]pyrimidine (XIV) (50 g, 1.0 equiv), (4-cyclopropyl-6-methoxypyrimidin-5-yl)boronic acid XXI (27.7 g, 1.2 equiv), and (dppf)PdCl₂ (6.52 g, 7.5 mol %) were charged, followed by degassed 1,4-dioxane (16 vol) at 20-25° C. under a nitrogen atmosphere. The resulting mixture was further degassed using three vacuum/nitrogen cycles. After heating the reaction mixture to 35-40° C., Cs₂CO₃ (3 equiv) in degassed DI water (4 vol) was added dropwise. The reaction temperature was raised to 75° C. and stirring was continued until the reaction was completed.

Following the completion of the reaction, the reaction mixture was cooled to 20-25° C., filtered through a Celite pad, and washed sequentially with ethyl acetate (2 vol). The solvent was then removed under reduced pressure. All volume references that follow are with respect to the crude compound VI obtained after concentration.

Crude (VI) was dissolved in ethyl acetate (10 vol) and the organic layer was washed with water (10 vol×2) then brine (10 vol). Activated charcoal was charged (Acticarbone® ENO-PC, 10% w/w) as a slurry in ethyl acetate (3 vol) and the reaction mixture was stirred for 2 hours at 30±5° C. The reaction mixture was filtered through a Celite pad, and rinsed through with ethyl acetate (3 vol). The carbon treatment above was then repeated.

Following the second carbon treatment, the solvent was removed at 60° C. to obtain a solid. The solid was recrystallized by adding ethyl acetate (3 vol) and heating to 78° C. for 1 hour. To the batch was slowly added n-heptane (7 vol) to the reaction mixture at 70-75° C., and the resulting mixture was stirred for 1 hour at that temperature. The mixture was cooled to 25° C. over a period of 3-4 hours then stirred for 12 hours at that temperature. The slurry filtered and washed with n-heptane (2 vol), then dried under vacuum overnight at 50-55° C. to obtain 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (VI) as a light yellow solid. (43 g, 67.7% yield; 98.8% AUC by HPLC). LC-MS (ESI+): m/z 535.2 [M+H]⁺; ¹H NMR (400 MHz, CDCl₃) δ 9.35 (s, 1H), 8.69 (s, 1H), 8.24 (s, 1H), 7.57-7.47 (m, 4H), 7.40 (d, J=1.4 Hz, 1H), 5.77 (s, 2H), 4.50 (hept, J=6.7 Hz, 1H), 3.94 (s, 3H), 1.69 (tt, J=8.3, 4.6 Hz, 1H), 1.43 (d, J=6.7 Hz, 6H), 1.30-1.20 (m, 2H), 0.89 (dt, J=8.1, 3.4 Hz, 2H).

### Example 14: Synthesis of 6-cyclopropyl-5-(1-(4-(1-isopropyl-4-(trifluoromethyl)-1H-imidazol-2-yl)benzyl)-1H-pyrazolo[3,4-d]pyrimidin-6-yl)pyrimidin-4-ol (XXV)

A solution of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (VI) (10 g, 1 equiv) in 2 N HCl (10 vol) was heated to 65-70° C. and stirred for 3 h. After completion, the reaction was cooled to 20-25° C. and washed with EtOAc (5 vol). The aqueous layer pH was adjusted to 7.5-8.0 with 10% aq. sodium carbonate solution (5 vol), then the product was extracted with EtOAc (2×10 vol). The organic layer was distilled under vacuum at 50° C. and the solids were suspended in ethyl acetate (1 vol) and n-heptane (2 vol). The resulting slurry was heated to 50-55° C., stirred for 1 hour and then cooled to room temperature. The solids were filtered, then dried under vacuum at 55-60° C. to obtain 6-cyclopropyl-5-(1-(4-(1-isopropyl-4-(trifluoromethyl)-1H-imidazol-2-yl)benzyl)-1H-pyrazolo[3,4-d]pyrimidin-6-yl)pyrimidin-4-ol (XXV) (7 g, 72%, 97.4% AUC by HPLC). LC-MS (ESI+): m/z 521.0 [M+H]⁺; ¹H NMR (400 MHz, DMSO-d₆): δ 12.61 (br s, 1H), 9.47 (s, 1H), 8.49 (s, 1H), 8.16 (d, J=4.9 Hz, 2H), 7.53-7.51 (m, 2H), 7.42-7.40 (m, 2H), 5.78 (s, 2H), 4.45-4.39 (m, 1H), 1.59-1.46 (m, 1H), 1.37 (d, J=6.7 Hz, 6H), 1.01-0.99 (m, 2H), 0.78-0.75 (m, 2H).

### Example 15: Synthesis of 6-(4-cyclopropyl-6-methoxypyrimidin-5-yl)-1-(4-(1-isopropyl-4-(trifluoromethyl)-1H-imidazol-2-yl)benzyl)-4,5-dihydro-1H-pyrazolo[3,4-d]pyrimidine (XXVI)

To a solution of 6-(4-cyclopropyl-6-methoxypyrimidin-5-yl)-1-(4-(1-isopropyl-4-(trifluoromethyl)-1H-imidazol-2-yl)benzyl)-1H-pyrazolo[3,4-d]pyrimidine (VI) (10 g, 1 equiv) in THE (5 vol) was charged sodium borohydride (5 equiv) and stirred for 15 minutes. The reaction mixture was cooled to 0-5° C. and acetic acid (0.1 vol.) was added dropwise. After reaction completion, the batch was basified with sodium bicarbonate and the layers were separated. The aqueous layer was extracted with ethyl acetate (10 vol), then the organic layers were combined and washed with brine (10 vol). The solvent was removed under vacuum and the obtained crude was purified by column chromatography using neutral alumina (80 to 90% of EtOAc/n-heptane). The product obtained from the column chromatography was slurried in ethyl acetate (1 vol) and n-heptane (2 vol) at 50-55° C., stirred for 1 hour and then cooled to room temperature. Solids were filtered and dried under vacuum to obtain 6-(4-cyclopropyl-6-methoxypyrimidin-5-yl)-1-(4-(1-isopropyl-4-(trifluoromethyl)-1H-imidazol-2-yl)benzyl)-4,5-dihydro-1H-pyrazolo[3,4-d]pyrimidine (XXVI) (9 g, 89%, 98% AUC by HPLC). LC-MS (ESI+): m/z 537.2 [M+H]+; ¹H NMR (400 MHz, DMSO-d₆): δ 8.62 (s, 1H), 8.16 (s, 1H), 7.98 (s, 1H), 7.50 (d, J=8.2 Hz, 2H), 7.32 (d, J=8.2 Hz, 2H), 7.15 (s, 1H), 5.25 (s, 2H), 4.74 (s, 2H), 4.44-4.41 (m, 1H), 3.93 (s, 3H), 2.18-2.08 (m, 1H), 1.47-1.32 (m, 6H), 1.03-1.01 (m, 2H), 0.98-0.87 (m, 2H).

### Example 16: Synthesis of 6-chloro-2-(4-(1-isopropyl-4-(trifluoromethyl)-1H-imidazol-2-yl)benzyl)-2H-pyrazolo[3,4-d]pyrimidine (XVII)

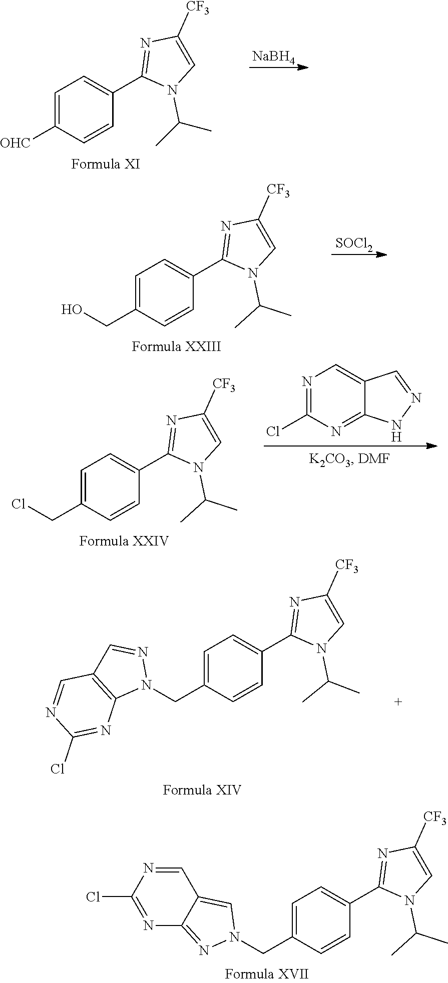

Step 1: To a solution of aldehyde (XI) (5 g, 1.0 eq.) in methanol (10 Vol) is added sodium borohydride (3.35 g, 5 equiv) at 0° C. and the progress of the reaction was monitored by TLC (ethyl acetate/hexane=1:1). After completion of the reaction, the batch was poured into water (20 vol) and allowed to stir for 3 h. The precipitated solids were filtered and dried under vacuum at 60° C. to provide alcohol (XXIII) (4.5 g, 89.3% yield, 96.3% AUC by HPLC).

Step 2: To a mixture of alcohol (XXIII) (4 g, 1 equiv) in 1,2-dichloroethane (8 vol) at room temperature was added thionyl chloride (3 equiv), and the reaction was stirred at that temperature for 4 h. Upon reaction completion, n-heptane (10 vol) was added to the reaction mixture, and allowed to stir for 2 h. The precipitated solids were filtered and dried under vacuum at room temperature to provide benzylic chloride (XXIV) (4.25 g, 99%, 97.8% purity by HPLC).

Step 3: To a solution of benzylic chloride (XXIV) (1 g, 1 equiv) in DMF (10 mL, 10 vol), was added 6-chloropyrazolopyrimidine (2.06 g, 1.05 equiv) and K₂CO₃ (1.37 g, 3.0 equiv). After 8 hours of stirring at room temperature, the mixture was quenched with water and extracted with ethyl acetate (100 mL×2). The combined organic phase was washed with water and brine, dried over Na₂SO₄, filtered and concentrated in vacuo. The resulting residue was purified by silica gel chromatography (petroleum ether: ethyl acetate=8:3) to afford both regioisomers of Formulae (XIV) and (XVII) in a 1.2:1 ratio.

Characterization data for Formula XVII: LC-MS (ESI+): m/z 421.3 [M+H]⁺; ¹H NMR (400 MHz, DMSO) δ 9.46 (s, 1H), 9.06 (s, 1H), 8.18 (s, 1H), 7.60 (d, J=8.4 Hz, 2H), 7.51 (d, J=8.4 Hz, 2H), 5.85 (s, 2H), 4.43 (hept, 1H), 1.36 (d, 6H).

### Example 17: 6-(4-cyclopropyl-6-methoxypyrimidin-5-yl)-2-(4-(1-isopropyl-4-(trifluoromethyl)-1H-imidazol-2-yl)benzyl)-2H-pyrazolo[3,4-d]pyrimidine (XX)

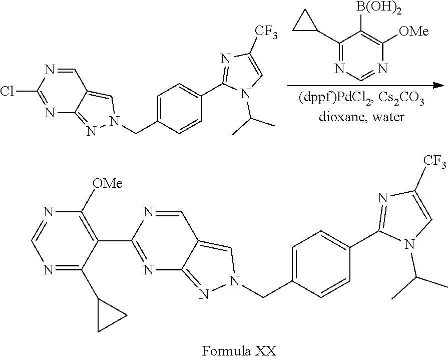

To a solution of Formula (XVII) (20 g, 1 eq) in 1,4-dioxane (16 vol) and water (4 vol), was added (4-cyclopropyl-6-methoxypyrimidin-5-yl)boronic acid (XXI, 2 equiv) and the batch was purged with argon gas for 45 minutes. To the batch was charged cesium carbonate (3 equiv) followed by Pd(dppf)Cl₂ (0.075 equiv) at room temperature and the batch was purged with argon for 15 min.

The batch was heated to 65±5° C. for 2 h. After consumption of the starting material, the batch was cooled to room temperature. The reaction mixture was filtered through a hyflo bed and the bed was washed with ethyl acetate (3 vol). The filtrate was concentrated up to 2.5±0.5 volumes at ≤60° C. under reduced pressure. The batch was diluted with ethyl acetate (10 vol) and washed with water (10 vol). The aqueous layer was re-extracted with ethyl acetate (10 vol). The combined organic layers were washed with 10% sodium chloride solution (10 vol), then concentrated to dryness. The crude product was purified by column chromatography using neutral alumina by eluting the product at 50 to 80% of ethyl acetate/n-heptane to obtain Formula (XX) (10 g, 40%, 99.2% AUC by HPLC) as a pale yellow solid. LC-MS (ESI+): m/z 535.0 [M+H]⁺.

### Example 18: Synthesis of 6-chloro-1-(4-(1-isopropyl-4-(trifluoromethyl)-1H-imidazol-2-yl)benzyl)-1H-pyrazolo[3,4-d]pyrimidine (XIV) from tert-butyl 2-(4-(1-isopropyl-4-(trifluoromethyl)-1H-imidazol-2-yl)benzyl)hydrazine-1-carboxylate (X)

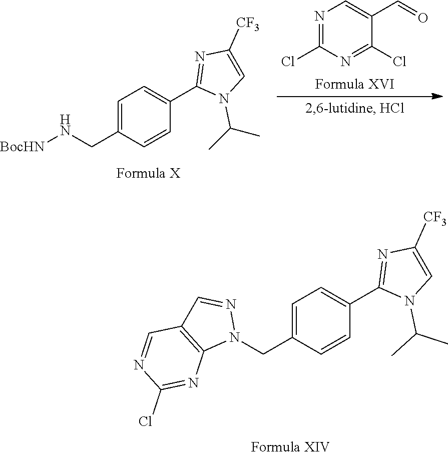 [SMILES: CC(C)n1cc(C(F)(F)F)nc1-c1ccc(CNNC(=O)OC(C)(C)C)cc1 ; O=Cc1cnc(Cl)nc1Cl ; CC(C)n1cc(C(F)(F)F)nc1-c1ccc(Cn2ncc3cnc(Cl)nc32)cc1]

Formula (X) (10.0 g, 1.0 eq), isopropanol (200 mL, 20 vol) and 2,6-lutidine (3.0 g, 1.1 eq) were charged to a round bottom flask to form a reaction mixture and cycled with nitrogen/vacuum three times. Formula (XVI) (4.9 g, 1.1 eq) was added into reaction mixture in portions as a solid at 20-25° C. The reaction mixture was then heated to 50-55° C. and stirred for 16 hours. HPLC was used to monitor the progress of the reaction. Concentrated HCl (4.9 g, 2.0 eq.) was then added to the mixture and stirred for 4 more hours to complete the Boc deprotection and condensation to form the pyrazolopyrimidine core.

The reaction mixture was cooled to 20-25° C. then concentrated to dryness at 40-45° C. under reduced pressure using a rotary evaporator. The crude compound was partitioned with ethyl acetate (100 mL) and water (100 mL) and stirred for 30 mins. The aqueous phase was further extracted with ethyl acetate (50 mL). The combined organic layers were washed with brine and dried at 40-45° C. under reduced pressure to obtain a solid (11.9 g). The solid was purified by column chromatography (silica gel; hexane/ethyl acetate, 3:1) to give Formula (XIV) (3.5 g, 33.2% yield) as a pale-yellow solid. (96.5% by HPLC).

### Example 19: Synthesis of tert-butyl 2-(4-(4-(trifluoromethyl)-1H-imidazol-2-yl)benzyl)hydrazine-1-carboxylate (XXVIII)

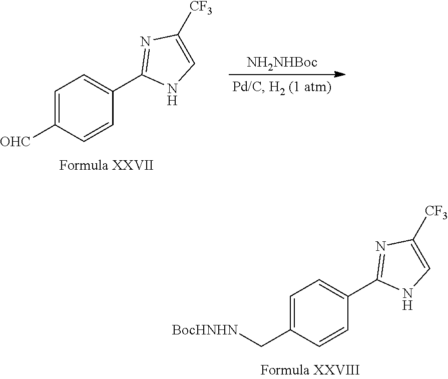

A 500 mL flask was purged with N₂, then Formula XXVII (20 g, 1.0 equiv) and EtOH (200 mL, 10 vol) was charged, and agitation was started. Tert-butyl carbazate (11.5 g, 1.05 equiv) and acetic acid (0.5 g, 0.1 equiv) were charged. The reaction was then stirred for 2 h at 25° C. Triethylamine (0.84 g, 0.1 equiv) and 10% Pd/C (2.5 g, 12.5% w/w) were charged. The flask was then flushed with H₂ gas three times, and stirred under 1 atm H₂ at 25-30° C. for approximately 6 h. Upon reaction completion, the mixture was filtered through Celite and rinsed with EtOH (60 mL, 3 vol). The filtrate was collected and concentrated at approximately 45° C. under vacuum. The resulting material was dissolved in ethyl acetate (200 mL, 10 vol) then washed with water (20 g, 1 vol). The phases were separated. The organic phase was concentrated at approximately 45° C. and n-hexane (100.0 mL, 5 vol) was charged. The mixture was stirred for 1 h at 20-25° C. The mixture was filtered and the solid was rinsed with n-hexane (20 mL, 1 vol). The solid was dried at 45~45° C. under vacuum to give Formula XXVIII as a white solid (28.4 g, 96% yield, 98.3% purity by HPLC).

¹H NMR (400 MHz, DMSO) δ 13.13 (s, 1H), 8.27 (s, 1H), 7.93-7.87 (m, 3H), 7.46-7.39 (m, 2H), 4.87-4.80 (m, 1H), 3.91 (d, J=4.2 Hz, 2H), 1.38 (s, 9H)

### Example 20: Synthesis of 2-(4-(hydrazineylmethyl)phenyl)-4-(trifluoromethyl)-1H-imidazole hydrochloride (XXVIV) from tert-butyl 2-(4-(4-(trifluoromethyl)-1H-imidazol-2-yl)benzyl)hydrazine-1-carboxylate (XXVIII)

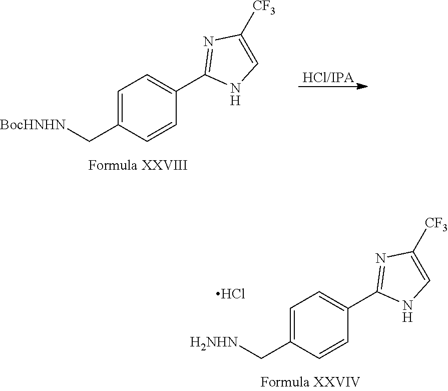

A 250 mL flask was purged with nitrogen, then Formula XXVIII (5 g, 1.0 equiv) and isopropanol (50 mL, 10 vol) were charged. The resulting solution was heated to 70-75° C. and HCl (4 M in isopropanol, 14.0 mL, 4.0 eq) was charged dropwise over at least 30 minutes. The reaction was kept stirring at 70-75° C. for at least 2 h, then cooled slowly to 20-25° C. The mixture was filtered, and the solid was rinsed with isopropanol (30 mL, 6 vol). The solid was dried at 45-50° C. under vacuum to give Formula XXVIV as a white solid (3.6 g, 78% yield, 99.2% purity by HPLC).

¹H NMR (400 MHz, DMSO) δ 13.43 (s, 1H), 8.05-7.98 (m, 2H), 7.92 (m, 1H), 7.55-7.49 (m, 2H), 4.10 (s, 2H)

### Example 21: Synthesis of 6-chloro-1-(4-(4-(trifluoromethyl)-1H-imidazol-2-yl)benzyl)-1H-pyrazolo[3,4-d]pyrimidine (XXX) from 2-(4-(hydrazineylmethyl)phenyl)-4-(trifluoromethyl)-1H-imidazole hydrochloride (XXVIV)

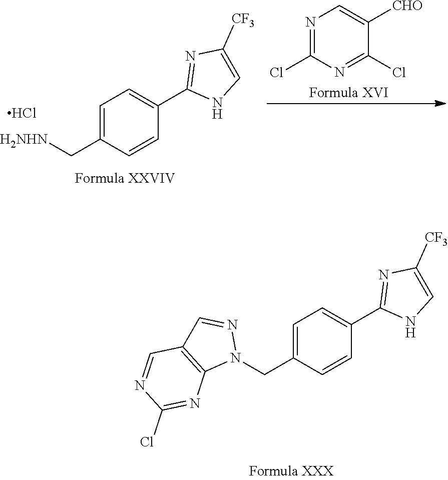

A 250 mL flask was purged with nitrogen, then Formula XXVIV (3 g, 1.0 equiv) and ethanol (60 mL, 20 vol) were charged, and agitation was started. The solution was cooled to approximately −10° C. Triethylamine (2.8 g, 3.0 equiv) was added dropwise. Then, Formula XVI (1.94 g, 1.2 equiv) was added in portions, keeping the internal temperature below −5° C. The reaction was stirred for 4 hours, then warmed to 20-25° C. The mixture was concentrated to dryness at 60° C., and to the residual material was charged dichloromethane (30 mL, 10 vol) and water (15 mL, 5 vol). The biphasic mixture was filtered, then the phases were separated. The organic phase was concentrated under vacuum at 40° C. to give a yellow solid (2.9 g, 82.9% yield, 87.1% purity by HPLC). An analytical sample (99% purity by HPLC) can be made by trituration with 10 vol of MeOH at 20-25° C.

¹H NMR (400 MHz, DMSO) δ 13.20 (s, 1H), 9.32 (s, 1H), 8.52 (s, 1H), 7.98-7.89 (m, 3H), 7.40-7.33 (m, 2H), 5.69 (s, 2H)

It is to be understood that the foregoing described embodiments and exemplifications are not intended to be limiting in any respect to the scope of the disclosure, and that the claims presented herein are intended to encompass all embodiments and exemplifications whether or not explicitly presented herein

All patents and publications cited herein are fully incorporated by reference in their entirety.

What is claimed is:

1. A compound of Formula I: 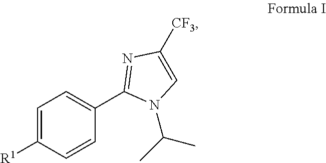
or a pharmaceutically acceptable salt thereof;
wherein
R¹ is selected from cyano, —CHO, —CH₂NR²R³, and —CH₂R⁴;
R² is selected from hydrogen, optionally substituted aryl, and optionally substituted heteroaryl;
R³ is selected from hydrogen, amino, and —NR⁵R⁶;
R⁴ is selected from Formula II and Formula III: 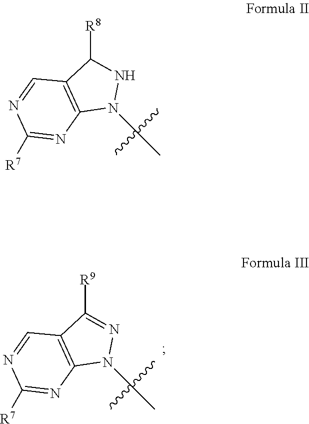
R⁵ and R⁶ are individually selected from hydrogen and an amine protecting group;
R⁷ is selected from optionally substituted aryl and optionally substituted heteroaryl;
R⁸ is selected from carbonyl, alkoxy, and sulfonate; and
R⁹ is selected from hydroxy, alkoxy, sulfonate, and a leaving group.

2. The compound of claim 1, or a pharmaceutically acceptable salt thereof, wherein one of R⁵ and R⁶ is hydrogen and the other of R⁵ and R⁶ is an amine protecting group selected from t-butyloxycarbonyl (Boc), fluorenylmethoxycarbonyl (Fmoc), benzyloxycarbonyl (Cbz), acetyl, trifluoroacetamide, phthalimide, benzyl, trityl, benzylideneamine, and toluenesulfonate.

3. The compound of claim 2, or a pharmaceutically acceptable salt thereof, wherein the amine protecting group is t-butyloxycarbonyl (Boc).

4. The compound of claim 1, or a pharmaceutically acceptable salt thereof, wherein R¹ is —CH₂NR²R³, R² is hydrogen, and R³ is amino.

5. The compound of claim 1, or a pharmaceutically acceptable salt thereof, wherein R¹ is —CH₂NR²R³ and R² is an optionally substituted pyrimidinyl.

6. The compound of claim 1, or a pharmaceutically acceptable salt thereof, wherein R⁷ has a Formula XIX: 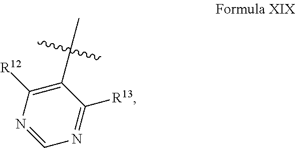
wherein R¹² and R¹³ are individually selected from hydrogen, hydroxy, alkyl, alkoxy, and cycloalkyl.

7. The compound of claim 6, or a pharmaceutically acceptable salt thereof, wherein R¹² is cyclopropyl and R¹³ is methoxy.

8. The compound of claim 1, or a pharmaceutically acceptable salt thereof, wherein the leaving group is selected from trifluoromethanesulfonate, toluenesulfonate, methanesulfonate, and halogen.

9. The compound of claim 8, or a pharmaceutically acceptable salt thereof, wherein the leaving group is trifluoromethanesulfonate.

10. The compound of claim 1, or a pharmaceutically acceptable salt thereof, selected from the group consisting of: 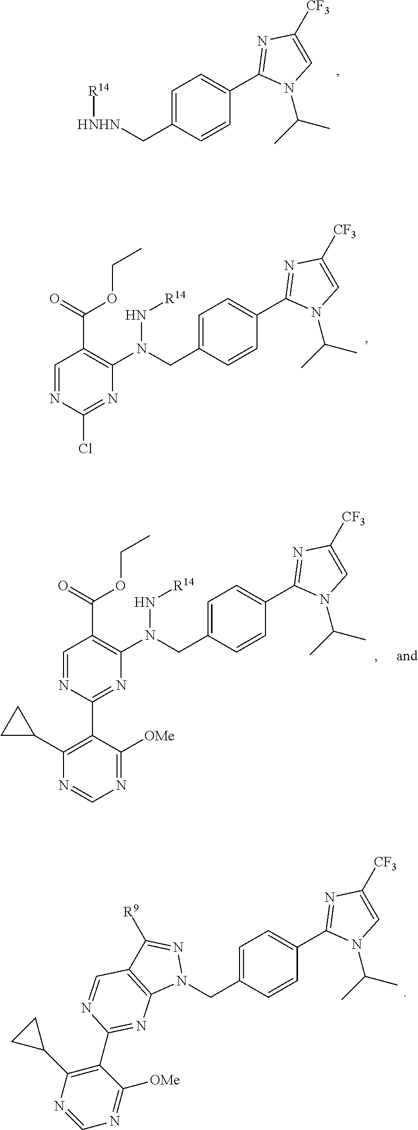
wherein R⁹ is a leaving group, and
wherein R¹⁴ is an amine protecting group.

11. The compound of claim 1, or a pharmaceutically acceptable salt thereof, selected from the group consisting of: 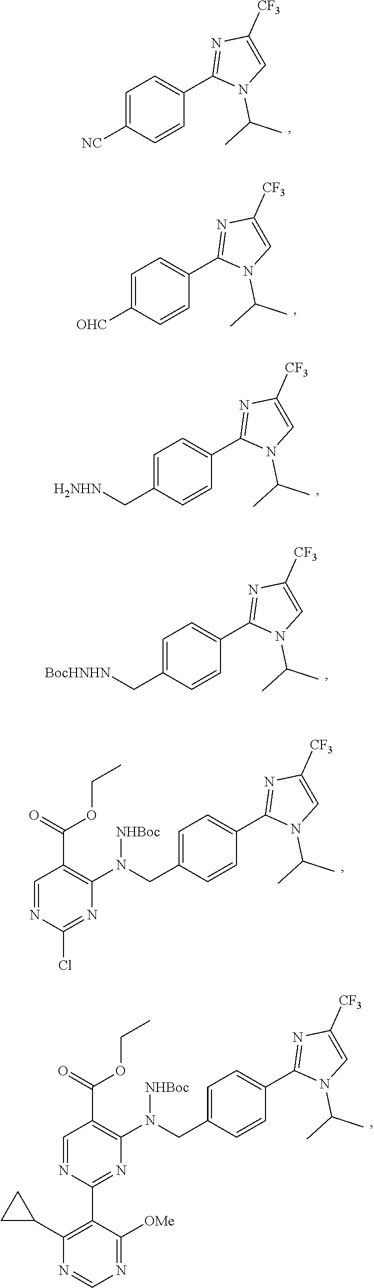 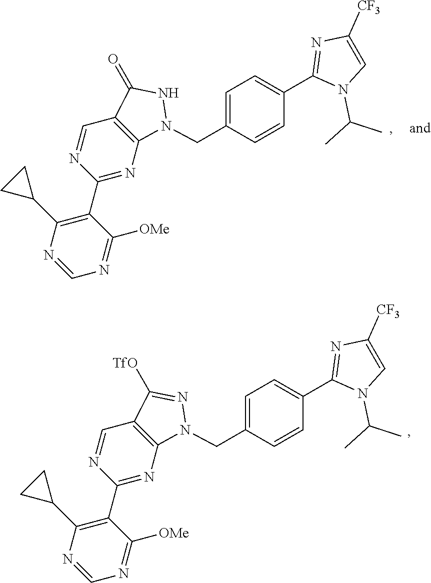 [SMILES: COc1ncnc(C2CC2)c1-c1ncc2c(=O)[nH]n(Cc3ccc(-c4nc(C(F)(F)F)cn4C(C)C)cc3)c2n1 ; COc1ncnc(C2CC2)c1-c1ncc2c(OS(=O)(=O)C(F)(F)F)nn(Cc3ccc(-c4nc(C(F)(F)F)cn4C(C)C)cc3)c2n1]
or a pharmaceutically acceptable salt thereof.

12. A compound of Formula IV: 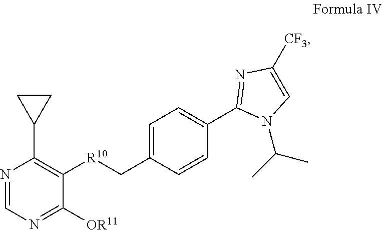
or a pharmaceutically acceptable salt thereof;
wherein 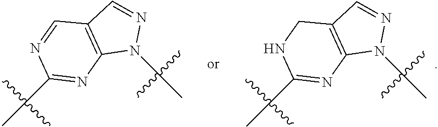 [SMILES: *c1ncc2cnn(*)c2n1 ; *C1=Nc2c(cnn2*)CN1]
and
R¹¹ is hydrogen or alkyl.

13. The compound of claim 12, or a pharmaceutically acceptable salt thereof, selected from

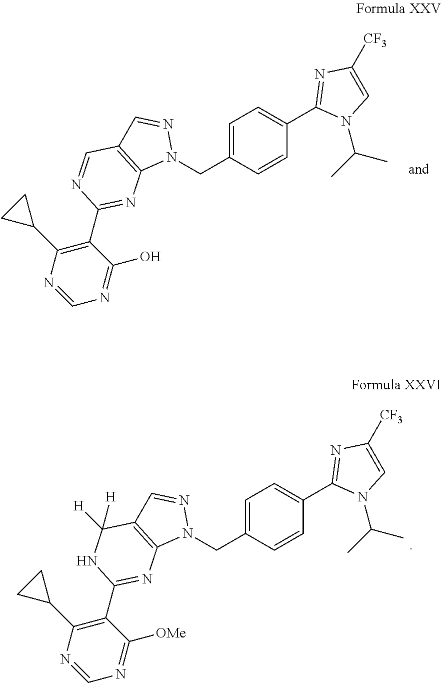 [SMILES: CC(C)n1cc(C(F)(F)F)nc1-c1ccc(Cn2ncc3cnc(-c4c(O)ncnc4C4CC4)nc32)cc1 ; COc1ncnc(C2CC2)c1C1=Nc2c(cnn2Cc2ccc(-c3nc(C(F)(F)F)cn3C(C)C)cc2)CN1]

14. A process for preparing a compound of Formula VI: 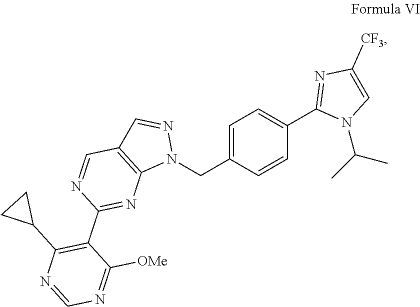 [SMILES: COc1ncnc(C2CC2)c1-c1ncc2cnn(Cc3ccc(-c4nc(C(F)(F)F)cn4C(C)C)cc3)c2n1]
comprising:
reducing a compound of Formula V:

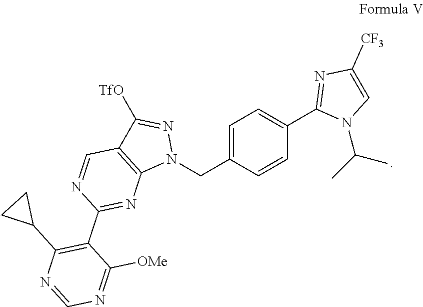 [SMILES: COc1ncnc(C2CC2)c1-c1ncc2c(OS(=O)(=O)C(F)(F)F)nn(Cc3ccc(-c4nc(C(F)(F)F)cn4C(C)C)cc3)c2n1]

15. The process of claim 14, wherein said reducing occurs in the presence of a palladium catalyst and a trialkylsilane.

16. The process of claim 14, wherein said reducing occurs in the presence of a palladium catalyst and triethylsilane.

17. The process of claim 14, further comprising:
mixing the compound of Formula VI with gentisic acid in a solvent to form a compound of Formula XVIII:

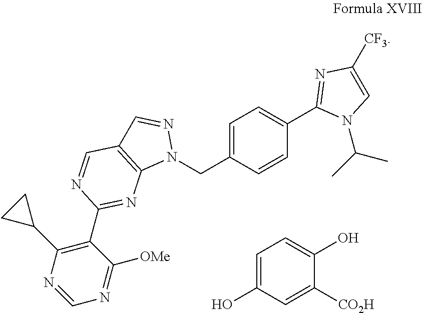 [SMILES: COc1ncnc(C2CC2)c1-c1ncc2cnn(Cc3ccc(-c4nc(C(F)(F)F)cn4C(C)C)cc3)c2n1 ; O=C(O)c1cc(O)ccc1O]

18. The process of claim 17, wherein the compound of Formula VI is mixed with gentisic acid in the presence of a solvent.

19. The process of claim 18, wherein the solvent is a mixture of isopropanol and heptane.

20. A process for preparing a compound of Formula Va: 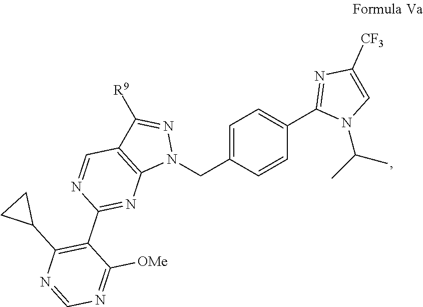
wherein R⁹ is a leaving group selected from trifluoromethanesulfonate, toluenesulfonate, methanesulfonate;
comprising:
reacting a compound of Formula VII: 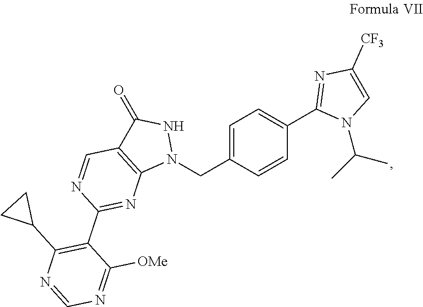 [SMILES: COc1ncnc(C2CC2)c1-c1ncc2c(=O)[nH]n(Cc3ccc(-c4nc(C(F)(F)F)cn4C(C)C)cc3)c2n1]
with toluenesulfonic anhydride, toluenesulfonyl chloride, methansulfonic anhydride, methansulfonyl chloride, trifluoromethanesulfonic anhydride, or trifluoromethanesulfonyl chloride to form a compound of Formula Va.

21. The process of claim 20, wherein the compound of Formula VII is reacted with trifluoromethanesulfonic anhydride or trifluoromethanesulfonyl chloride.

22. A process of preparing a compound of Formula VII: 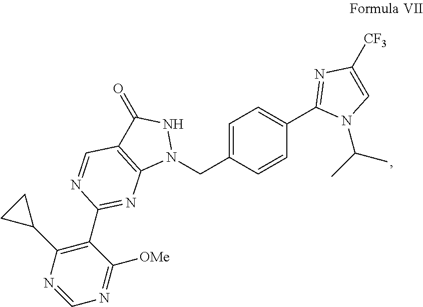 [SMILES: COc1ncnc(C2CC2)c1-c1ncc2c(=O)[nH]n(Cc3ccc(-c4nc(C(F)(F)F)cn4C(C)C)cc3)c2n1]
comprising:
reacting a compound of Formula VIII: 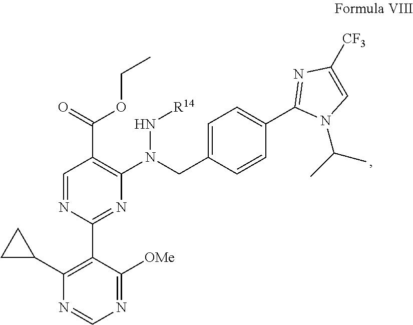
or a salt thereof,
with an acid to form a compound of Formula VII,
wherein R¹⁴ is an amine protecting group.

23. The process of claim 22, wherein the acid is trifluoroacetic acid.

24. The process of claim 22, wherein said reacting occurs in the presence of toluene as a solvent.

25. The process of claim 22, wherein said reacting occurs at a temperature of from about 20° C. to about 55° C.

26. A process of preparing a compound of Formula VIII: 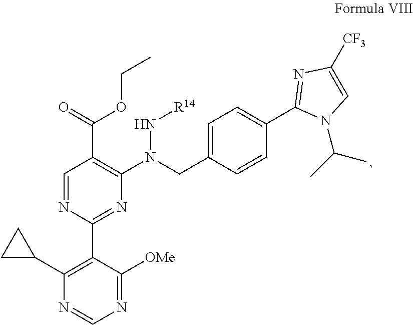
or a salt thereof,
comprising:
coupling a compound of Formula IX: 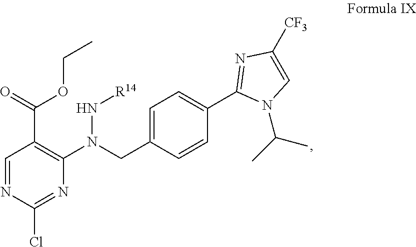
or a salt thereof,
with (4-cyclopropyl-6-methoxypyrimidin-5-yl)boronic acid to form a compound of Formula VIII,
wherein R¹⁴ is an amine protecting group.

27. The process of claim 26, wherein said coupling occurs in the presence of a palladium catalyst.

28. The process of claim 26, wherein said coupling occurs in the presence of a solvent selected from 1,4-dioxane, water, and mixtures thereof.

29. The process of claim 26, wherein said coupling occurs at a temperature of from about 20° C. to about 65° C.

30. A process of preparing a compound of Formula IX: 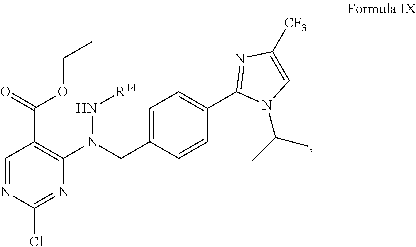
or a salt thereof,
comprising:
reacting a compound of Formula X: 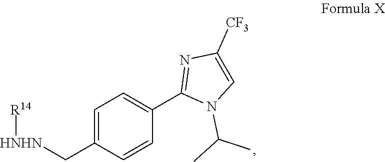
or a salt thereof,
with a 2,4-dichloropyrimidine-5-carboxylate ester to form a compound of Formula IX,
wherein R¹⁴ is an amine protecting group.

31. The process of claim 30, wherein the 2,4-dichloropyrimidine-5-carboxylate ester is ethyl 2,4-dichloropyrimidine-5-carboxylate.

32. The process of claim 30, wherein the 2,4-dichloropyrimidine-5-carboxylate ester is isopropyl 2,4-dichloropyrimidine-5-carboxylate.

33. The process of claim 30, wherein the compound of Formula X is reacted with the 2,4-dichloropyrimidine-5-carboxylate ester in the presence of a base and a solvent.

34. The process of claim 33, wherein the base is an amine base, and the solvent is an alcohol solvent.

35. The process of claim 34, wherein the amine base is 2,6-lutidine.

36. The process of claim 34, wherein the solvent is isopropanol.

37. The process of any of claims 22-36, wherein the amine protecting group is t-butyloxycarbonyl (Boc).

38. A process of preparing a compound of Formula X: 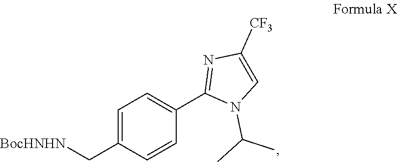
or a salt thereof,
comprising:
reacting a compound of Formula XI: 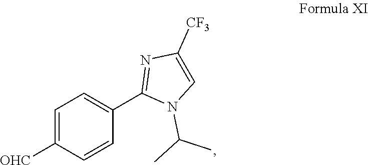 [SMILES: CC(C)n1cc(C(F)(F)F)nc1-c1ccc(C=O)cc1]
with tert-butyl carbazate and a reducing agent to form a compound of Formula X.

39. The process of claim 38, wherein the compound of Formula XI is reacted with tert-butyl carbazate in the presence of sodium cyanoborohydride.

40. The process of claim 38, wherein the compound of Formula XI is reacted with tert-butyl carbazate in the presence of hydrogen and a transition metal catalyst.

41. A process of preparing a compound of Formula XI: 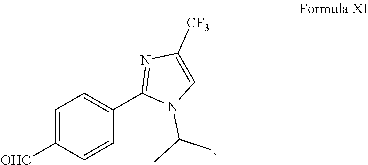 [SMILES: CC(C)n1cc(C(F)(F)F)nc1-c1ccc(C=O)cc1]
comprising:
reacting a compound of Formula XII: 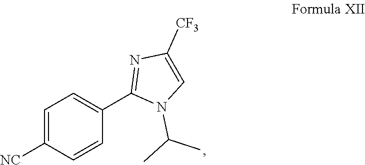 [SMILES: CC(C)n1cc(C(F)(F)F)nc1-c1ccc(C#N)cc1]
with a reducing agent to form a compound of Formula XI.

42. The process of claim 41, wherein the reducing agent is diisobutylaluminum hydride.

43. A process of preparing a compound of Formula XII: 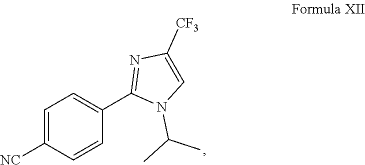 [SMILES: CC(C)n1cc(C(F)(F)F)nc1-c1ccc(C#N)cc1]
comprising:
reacting a compound of Formula XIII: 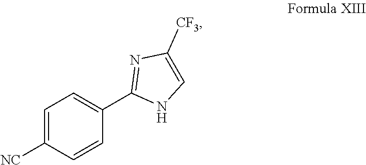 [SMILES: N#Cc1ccc(-c2nc(C(F)(F)F)c[nH]2)cc1]
with an alkylating agent to form a compound of Formula XII.

44. The process of claim 43, wherein the alkylating agent is selected from 2-iodopropane, 2-bromopropane, 2-isopropyl mesylate, and 2-isopropyl tosylate.

45. The process of claim 43, wherein the alkylating agent is 2-iodopropane.

46. A process of preparing a compound of Formula XIV: 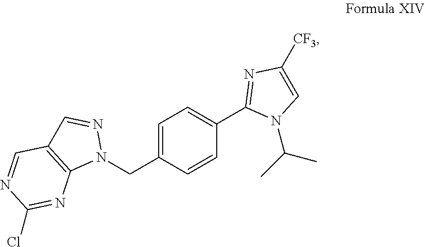 [SMILES: CC(C)n1cc(C(F)(F)F)nc1-c1ccc(Cn2ncc3cnc(Cl)nc32)cc1]
comprising:
reacting a compound of Formula XV: 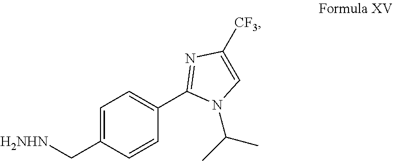
or a salt thereof,
with a compound of Formula XVI: 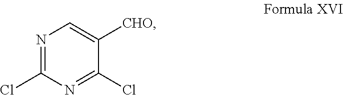
to form a compound of Formula XIV.

47. The process of claim 46, wherein the compound of Formula XV is reacted with the compound of Formula XVI in the presence of a solvent and a base.

48. The process of claim 47, wherein the solvent is selected from ethanol, isopropanol, and tetrahydrofuran.

49. The process of claim 47, wherein the base is an amine base.

50. The process of claim 49, wherein the base is selected from triethylamine and diisopropylethylamine.

51. The process of claim 46, wherein the compound of Formula XV is reacted with the compound of Formula XVI at a temperature of from −20° C. to 25° C.

52. The process of claim 46, wherein the reaction of the compound of Formula XV with the compound of Formula XVI also forms a compound of Formula XVII.

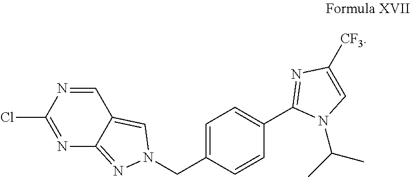 [SMILES: CC(C)n1cc(C(F)(F)F)nc1-c1ccc(Cn2cc3cnc(Cl)nc3n2)cc1]

53. The process of claim 52, wherein the compound of Formula XVII forms in an amount of less than 5% AUC as compared to the area of the compound of Formula XIV.

54. The process of claim 52, wherein the compound of Formula XIV is isolated containing less than 1%, less than 0.5%, less than 0.25%, or less than 0.15% AUC of the compound of Formula XVII.

55. The process of claim 46, further comprising reacting the compound of Formula XIV with a compound of Formula XXI 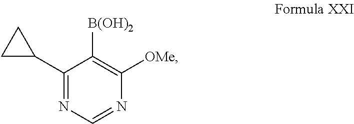 [SMILES: COc1ncnc(C2CC2)c1B(O)O]
to obtain a compound of Formula VI

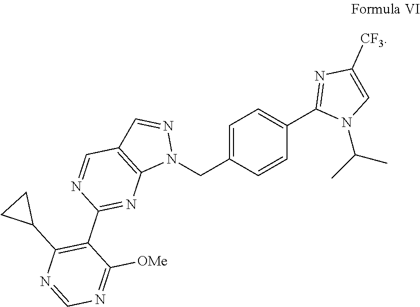 [SMILES: COc1ncnc(C2CC2)c1-c1ncc2cnn(Cc3ccc(-c4nc(C(F)(F)F)cn4C(C)C)cc3)c2n1]

56. The process of claim 55, further comprising mixing the compound of Formula VI with gentisic acid in a solvent to form a compound of Formula XVIII:

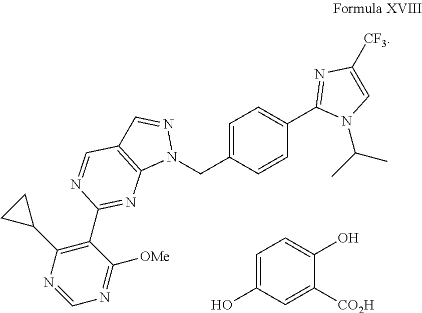 [SMILES: COc1ncnc(C2CC2)c1-c1ncc2cnn(Cc3ccc(-c4nc(C(F)(F)F)cn4C(C)C)cc3)c2n1 ; O=C(O)c1cc(O)ccc1O]

57. A compound of Formula XIV 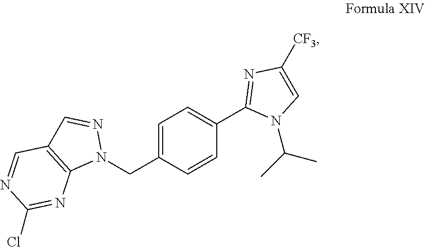 [SMILES: CC(C)n1cc(C(F)(F)F)nc1-c1ccc(Cn2ncc3cnc(Cl)nc32)cc1]
or a salt thereof, prepared by the process of claim 47.

58. A compound of Formula XVII: 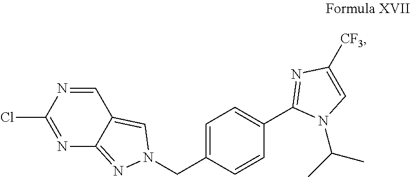 [SMILES: CC(C)n1cc(C(F)(F)F)nc1-c1ccc(Cn2cc3cnc(Cl)nc3n2)cc1]
or a salt thereof.

59. A compound of Formula XX: 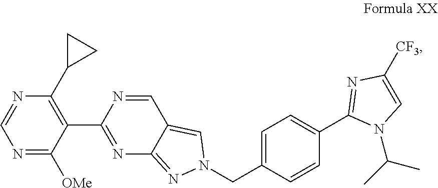 [SMILES: COc1ncnc(C2CC2)c1-c1ncc2cn(Cc3ccc(-c4nc(C(F)(F)F)cn4C(C)C)cc3)nc2n1]
or a salt thereof.

60. A compound of Formula XXVIII: 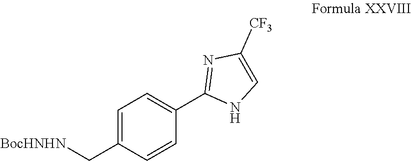
or a salt thereof.

61. A compound of Formula XXVIV: 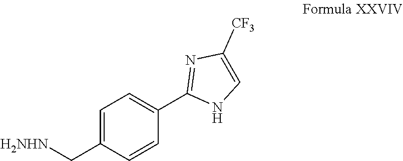
or a salt thereof.

62. A compound of Formula XXX: 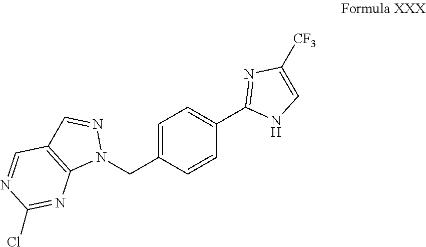 [SMILES: FC(F)(F)c1c[nH]c(-c2ccc(Cn3ncc4cnc(Cl)nc43)cc2)n1]
or a salt thereof.

63. A process of preparing a compound of Formula XIV: 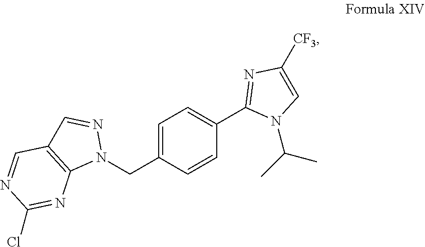 [SMILES: CC(C)n1cc(C(F)(F)F)nc1-c1ccc(Cn2ncc3cnc(Cl)nc32)cc1]
comprising:
reacting a compound of Formula X: 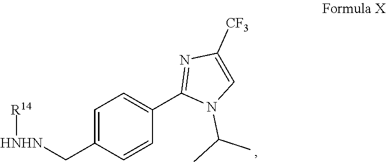
or a salt thereof,
with a compound of Formula XVI: 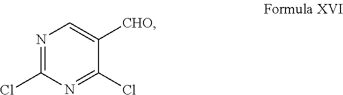
to form a compound of Formula XIV;
wherein R¹⁴ is an amine protecting group.

64. A process of preparing a compound of Formula XXX: 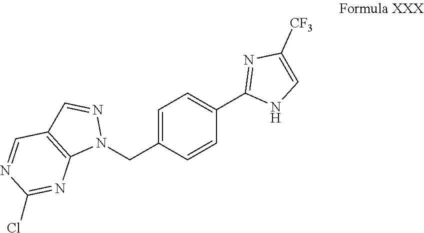 [SMILES: FC(F)(F)c1c[nH]c(-c2ccc(Cn3ncc4cnc(Cl)nc43)cc2)n1]
comprising:
reacting a compound of Formula XXVIV 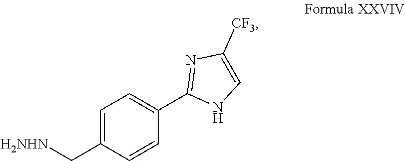
or a salt thereof,
with a compound of Formula XVI: 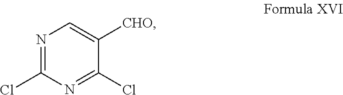
to form a compound of Formula XXX.

65. The process of claim 64, wherein the compound of Formula XXVIV is reacted with the compound of Formula XVI in the presence of a solvent and a base.

66. The process of claim 65, wherein the solvent is selected from ethanol, isopropanol, or THF.

67. The process of claim 65, wherein the base is an amine base.

68. The process of claim 67, wherein the base is selected from triethylamine and diisopropylethylamine.

69. The process of claim 64, wherein the reaction is conducted at a temperature of between −20° C. and 25° C.

70. A process of preparing a compound of Formula XXVIV: 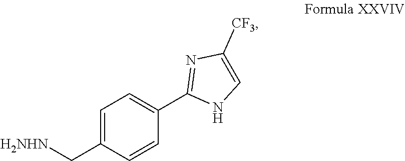
or a salt thereof,
comprising:
reacting a compound of Formula XXVIII 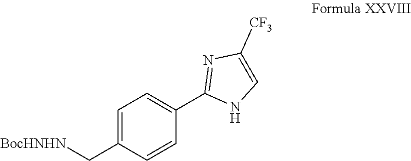
with acid to form a compound of Formula XXVIV or a salt thereof.

71. The process of claim 70, wherein the acid is selected from hydrochloric acid (HCl), hydrobromic acid (HBr), methanesulfonic acid, toluenesulfonic acid, trifluoroacetic acid, and trifluoromethanesulfonic acid.

72. The process of claim 70, wherein the reaction is conducted at a temperature of between between 0 and 75° C.

73. A process of preparing a compound of Formula XXVIII: 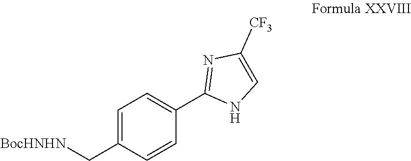
or a salt thereof,
comprising:
reacting a compound of Formula XXVII: 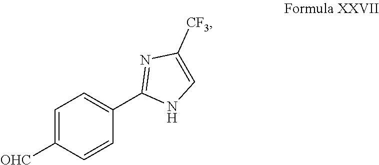 [SMILES: O=Cc1ccc(-c2nc(C(F)(F)F)c[nH]2)cc1]
with tert-butyl carbazate and a reducing agent to form a compound of Formula XXVIII.

74. The process of claim 73, wherein the compound of Formula XXVII is reacted with tert-butyl carbazate in the presence of hydrogen and a transition metal catalyst.

75. The process of claim 64, further comprising reacting the compound of Formula XXX with a compound of Formula XXI: 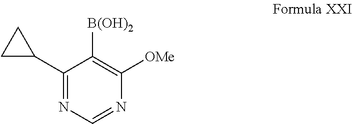 [SMILES: COc1ncnc(C2CC2)c1B(O)O]
to form a compound of Formula XXXI:

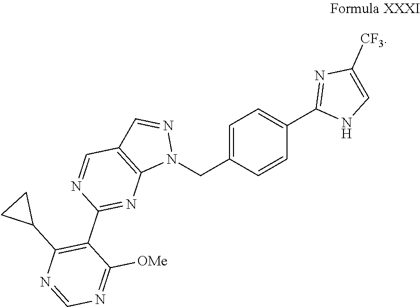 [SMILES: COc1ncnc(C2CC2)c1-c1ncc2cnn(Cc3ccc(-c4nc(C(F)(F)F)c[nH]4)cc3)c2n1]

76. The process of claim 75, further comprising reacting the compound of Formula XXXI with 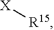
wherein X is a leaving group and R¹⁵ is an alkyl group;
to form a compound of Formula XXXII: 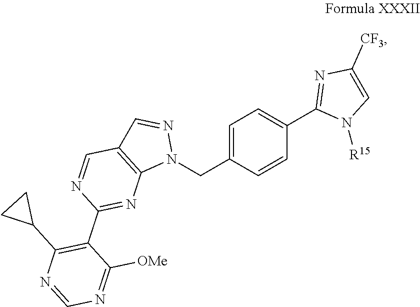
or a salt thereof.

77. The process of claim 76, wherein R¹⁵ is an isopropyl group.

78. The process of claim 76, wherein R¹⁵ is a methyl group.
